(19) United States
(12) Patent Application Publication
(10) Pub. No.: US 2025/0381291 A1
(43) Pub. Date: Dec. 18, 2025

(54) ANTI-CEACAM5 ANTIBODIES, ANTIBODY-DRUG CONJUGATES AND METHODS OF USES THEREOF

(71) Applicant: SEAGEN INC., Bothell, WA (US)

(72) Inventors: Travis Biechele, Eastsound, WA (US); Amanda L. Blasius, Bothell, WA (US); Jamie Apperson Mitchell, Bothell, WA (US); Albina Nesterova, Seattle, WA (US); Rory Rohm, Monroe, WA (US)

(21) Appl. No.: 19/234,840

(22) Filed: Jun. 11, 2025

Related U.S. Application Data
(60) Provisional application No. 63/659,116, filed on Jun. 12, 2024.

(51) Int. Cl. A61K 47/68; A61P 35/00; C07K 16/28

(52) U.S. Cl. CPC A61K 47/6853; A61K 47/68031; A61P 35/00; C07K 16/2803; C07K 2317/33; C07K 2317/92; C07K 2317/94

(57) ABSTRACT

The present invention relates to antibodies that bind to CEACAM5 and antibody-drug conjugates thereof. The present invention also relates to compositions and methods and uses of antibodies that specifically bind to CEACAM5 and antibody-drug conjugates thereof.

### CROSS REFERENCE TO RELATED APPLICATIONS

This application claims the benefit of U.S. Provisional Application No. 63/659,116, filed Jun. 12, 2024, the disclosure of which is hereby incorporated by reference in its entirety.

### REFERENCE TO SEQUENCE LISTING

This application is being filed electronically via Patent Center and includes an electronically submitted sequence listing in xml format. The .xml file contains a sequence listing entitled “PC073130A Sequence Listing.xml” created on May 10, 2025 and having a size of 38,268 bytes. The sequence listing contained in this .xml file is part of the specification and is hereby incorporated by reference herein in its entirety.

### BACKGROUND

The present invention relates to antibodies that bind to human CEACAM5 (Carcinoembryonic Antigen Cell Adhesion Molecule 5), CEACAM5 antibody-drug conjugates (ADCs) and compositions, methods and uses thereof, including use of the antibodies or ADCs of the disclosure to treat cancer.

CEACAM5 (also known as CEA, CD66e) is a GPI linked protein that belongs to Carcinoembryonic Antigen Cell Adhesion Molecule (CEACAM) family. The primary CEACAM5 transcript encodes a protein with an Ig variable region (IgV)-like domain, termed N, followed by six Ig constant region (IgC)-type 2-like domains, termed A1, B1, A2, B2, A3, and B3. (Thompson J A et al., PNAS. 1987; 84:2965-69; Zimmermann W et al., PNAS, 1987, 84 (9), 2960-2964; Beauchemin N et al., Cancer Metastasis Rev. 2013. 32:643-671). It is released from the cell surface by phosphatidylinositol specific phospholipase C. CEACAM5 is a marker for colorectal tumor detection and is abundant in multiple other solid tumors, such as gastric, lung, esophageal, pancreatic, bladder, breast, ovarian and cervical. (Hammarstrom S et al., Amsterdam: Harwood Academic Publishers; 1998:1-30. 4; Hammarstrom S, Semin Cancer Biol. 1999; 9:67-81; Decary S et al., Clin Cancer Res 2020; 26; 6589-6599; Bechmann M B et al., Oncotarget. 2020 Oct. 27; 11 (43): 3886-3899; Powell E et al., NPJ Breast Cancer 2018 30:4; 9; Chevinsky A H, Semin Surg Oncol 1991; 7:162-6; Chao A et al., Int J cancer. 2006; 119 (10): 91-98; Mallmann M R et al., Cancers (Basel) 2024, 16 (9), 1787). CEACAM5 is thought to have a role in tumor progression. (Beauchemin N et al., Cancer Metastasis Rev. 2013. 32:643-671).

There is clearly a significant need for effective and/or improved treatments for cancer. The present invention meets this need, as well as providing related benefits.

### SUMMARY

The present disclosure provides antibodies that bind to human CEACAM5 and antibody-drug conjugates comprising these anti-CEACAM5 antibodies (CEACAM5-ADCs), as well as uses of the antibodies and ADCs and associated methods. The disclosure also provides processes for making, preparing, and producing antibodies that bind to CEACAM5 and CEACAM5-ADCs.

Methods of treatment using the antibodies are provided. Such methods include, but are not limited to, one or more of methods of treating or methods of preventing diseases associated with or mediated by CEACAM5 expression, such as cancer. Antibodies and ADCs of the disclosure are useful in one or more of diagnosis, prophylaxis, or treatment of disorders or conditions mediated by, or associated with, CEACAM5 activity, including, but not limited to cancer. The disclosure further encompasses expression of antibodies, and preparation and manufacture of compositions comprising antibodies and ADCs of the disclosure, such as medicaments for the use of the antibodies. Polynucleotides encoding antibodies that bind CEACAM5 are also provided. Polynucleotides encoding antibody heavy chains or light chains, or both are provided. Host cells that express the antibodies are provided.

In some embodiments, an isolated antibody that binds to human CEACAM5 and comprises a heavy chain variable region (VH) and a light chain variable region (VL), wherein: the VH CDR1 comprises the amino acid sequence of SEQ ID NO: 8, the VH CDR2 comprises the amino acid sequence of SEQ ID NO: 9, the VH CDR3 comprises the amino acid sequence of SEQ ID NO: 10, the VL CDR1 comprises the amino acid sequence of SEQ ID NO: 11, the VL CDR2 comprises the amino acid sequence of SEQ ID NO: 12, and the VL CDR3 comprises the amino acid sequence of SEQ ID NO: 13, is provided.

In some embodiments, the isolated antibody that binds to human CEACAM5 comprises a VH that comprises the amino acid sequence of SEQ ID NO: 14 or a variant of SEQ ID NO: 14 comprising one to four amino acid substitutions at residues that are not within a CDR, and the VL comprises the amino acid sequence of SEQ ID NO: 15 or a variant of SEQ ID NO: 15 thereof comprising one to four amino acid substitutions at residues that are not within a CDR. In some embodiments, the VH has at least 95% sequence identity to the amino acid sequence of SEQ ID NO: 14, and the VL has at least 95% sequence identity to the amino acid sequence of SEQ ID NO: 15. In yet other embodiments, the VH comprises the amino acid sequence of SEQ ID NO: 14, and the VL comprises the amino acid sequence of SEQ ID NO: 15.

Also provided herein is an isolated antibody that binds to human CEACAM5 and comprises a heavy chain variable region (VH) and a light chain variable region (VL), wherein: the VH CDR1, VH CDR2, and VH CDR3 comprises the VH CDR1, VH CDR2, and VH CDR3 amino acid sequences, respectively, of SEQ ID NO: 14; and the VL CDR1, VL CDR2, and VL CDR3 comprises the VL CDR1, VL CDR2, and VL CDR3 amino acid sequences, respectively, of SEQ ID NO: 15, wherein the CDRs are the Kabat defined CDRs, the Chothia-defined CDRs, or the AbM-defined CDRs.

The antibody disclosed herein can further comprise a heavy chain constant region. In some embodiments, the heavy chain constant region comprises the amino acid sequence of SEQ ID NO: 16 or SEQ ID NO: 20. In some embodiments, the C-terminal lysine of the heavy chain has been removed, such as a heavy chain constant region comprising the amino acid sequence of SEQ ID NO: 30 or 32.

The antibody disclosed herein can further comprises a light chain constant region. In some embodiments, the light chain constant region comprises the amino acid sequence of SEQ ID NO: 17 or SEQ ID NO: 21.

Also provided herein is an isolated antibody that binds to human CEACAM5 comprising: (a) a heavy chain comprising the amino acid sequence of SEQ ID NO: 18 or 31 (with or without the C-terminal lysine, respectively), and a light chain comprising the amino acid sequence of SEQ ID NO: 19; (b) a heavy chain comprising the amino acid sequence of SEQ ID NO: 18 or 31 (with or without the C-terminal lysine, respectively), and a light chain comprising the amino acid sequence of SEQ ID NO: 23; (c) a heavy chain comprising the amino acid sequence of SEQ ID NO: 22 or 33 (with or without the C-terminal lysine, respectively), and a light chain comprising the amino acid sequence of SEQ ID NO: 23; or (d) a heavy chain comprising the amino acid sequence of SEQ ID NO: 22 or 33 (with or without the C-terminal lysine, respectively), and a light chain comprising the amino acid sequence of SEQ ID NO: 19.

In some embodiments, the antibody that binds to human CEACAM5 does not bind to cynomolgus monkey CEACAM5. In some embodiments, the antibody does not bind the A2-B2 domains of human CEACAM5. In some embodiments, the antibody does not bind the A3-B3 domains of human CEACAM5. In some embodiments, the antibody binds the N, A1, A2 or N-A1-A2 domains of human CEACAM5.

Provided herein is also an isolated polynucleotide encoding an antibody disclosed herein. Also provided is a pharmaceutical composition comprising a therapeutically effective amount of an antibody disclosed herein, and a pharmaceutically acceptable carrier. In some embodiments, a pharmaceutical composition for the treatment of cancer, comprising an antibody disclosed herein is provided. In some embodiments, an anti-cancer agent comprises an antibody disclosed here. In some embodiments, use of an antibody disclosed herein can be used to treat cancer or in the manufacture of a medicament for the treatment of cancer.

In some embodiments, a method of treating a cancer in a subject is provided, wherein the method comprises administering to the subject in need thereof a therapeutically effective amount of a pharmaceutical composition or antibody disclosed herein. In some embodiments, an antibody disclosed herein is for use as a medicament. In some embodiments, an antibody disclosed herein is for the treatment of cancer.

Also provided herein are antibody drug conjugates (ADCs) comprising an anti-CEACAM5 antibody. In one embodiment, the CEACAM5 antibody-drug conjugate (CEACAM5-ADC) comprises an anti-CEACAM5 antibody conjugated to a vcMMAE (valine-citruline-monomethyl auristatin E), wherein the anti-CEACAM5 antibody comprises a heavy chain variable region (VH) and a light chain variable region (VL), wherein the VH-CDR1, VH-CDR2, VH-CDR3 and VL-CDR1, VL-CDR2, and VL-CDR3 sequences are SEQ ID NOs: 8-13, respectively; wherein the vcMMAE comprises the structure:

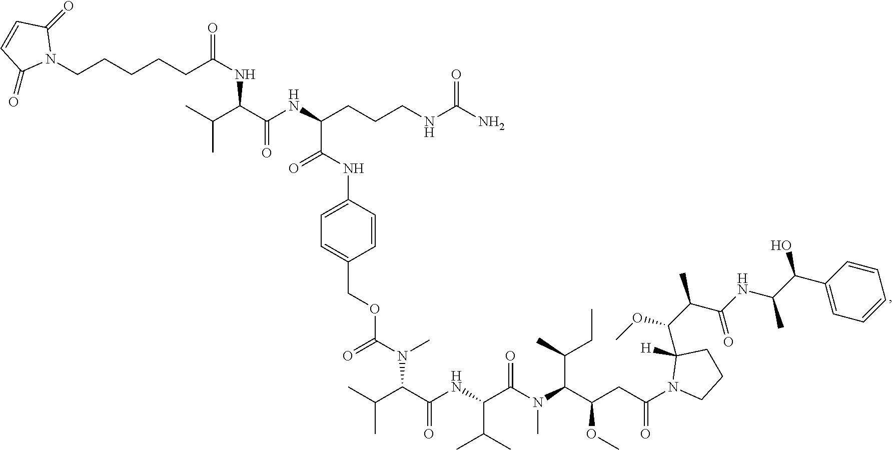 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

or a pharmaceutically acceptable salt thereof. In some embodiments, the anti-CEACAM5 antibody of the CEACAM5-ADC comprises: (a) a VH that comprises the amino acid sequence of SEQ ID NO: 14 or a variant of SEQ ID NO: 14 comprising one to four amino acid substitutions at residues that are not within a CDR, and (b) a VL comprises the amino acid sequence of SEQ ID NO: 15 or a variant of SEQ ID NO: 15 thereof comprising one to four amino acid substitutions at residues that are not within a CDR. In some embodiments, the VH has at least 95% sequence identity to the amino acid sequence of SEQ ID NO: 14, and the VL has at least 95% sequence identity to the amino acid sequence of SEQ ID NO: 15. In yet other embodiments, the VH comprises the amino acid sequence of SEQ ID NO: 14, and the VL comprises the amino acid sequence of SEQ ID NO: 15.

Also provided herein is a CEACAM5-ADC that comprises an anti-CEACAM5 antibody conjugated to a vcMMAE (valine-citruline-monomethyl auristatin E), wherein the anti-CEACAM5 antibody comprises a heavy chain variable region (VH) and a light chain variable region (VL), wherein (a) the VH CDR1, VH CDR2, and VH CDR3 comprises the VH CDR1, VH CDR2, and VH CDR3 amino acid sequences, respectively, of SEQ ID NO: 14; and (b) the VL CDR1, VL CDR2, and VL CDR3 comprises the VL CDR1, VL CDR2, and VL CDR3 amino acid sequences, respectively, of SEQ ID NO: 15; wherein the CDRs are the Kabat defined CDRs, the Chothia-defined CDRs, or the AbM-defined CDRs; and wherein the vcMMAE comprises the structure:

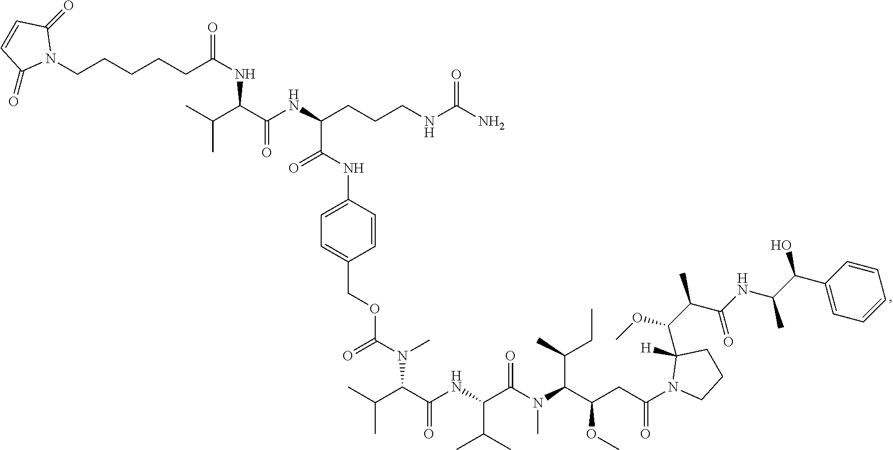 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

or a pharmaceutically acceptable salt thereof. In some embodiments, the antibody further comprises a heavy chain constant region, wherein the heavy chain constant region can comprise the amino acid sequence of SEQ ID NO: 16, 20, 30, or 32. In some embodiments, the antibody further comprises a light chain constant region, wherein the light chain constant region can comprise the amino acid sequence of SEQ ID NO: 17 or SEQ ID NO: 21.

Also provided herein is a CEACAM5-ADC that comprises an anti-CEACAM5 antibody conjugated to a vcMMAE (valine-citruline-monomethyl auristatin E), wherein the anti-CEACAM5 antibody comprises a heavy chain comprising the amino acid sequence of SEQ ID NO: 18 or 31 (with or without the C-terminal lysine, respectively), and a light chain comprising the amino acid sequence of SEQ ID NO: 19, and wherein the vcMMAE comprises the structure:

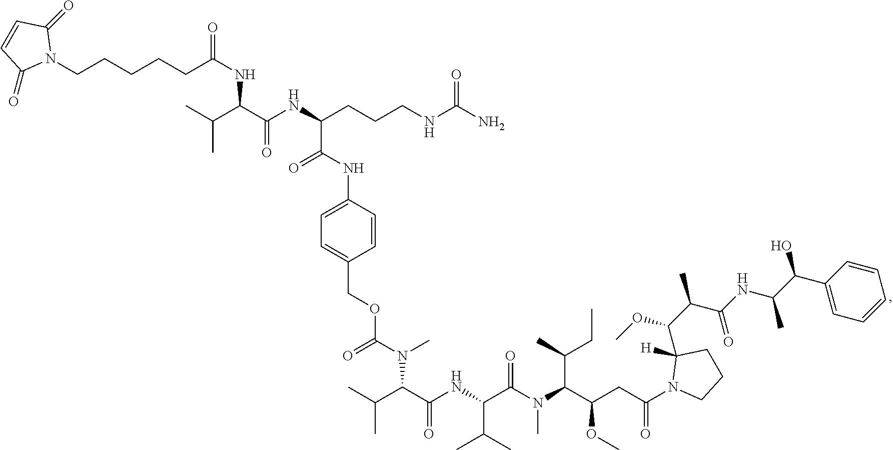 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

or a pharmaceutically acceptable salt thereof.

Also provided herein is a CEACAM5-ADC that comprises an anti-CEACAM5 antibody conjugated to a vcMMAE (valine-citruline-monomethyl auristatin E), wherein the anti-CEACAM5 antibody comprises a heavy chain comprising the amino acid sequence of SEQ ID NO: 18 or 31 (with or without the C-terminal lysine, respectively), and a light chain comprising the amino acid sequence of SEQ ID NO: 23, and wherein the vcMMAE comprises the structure:

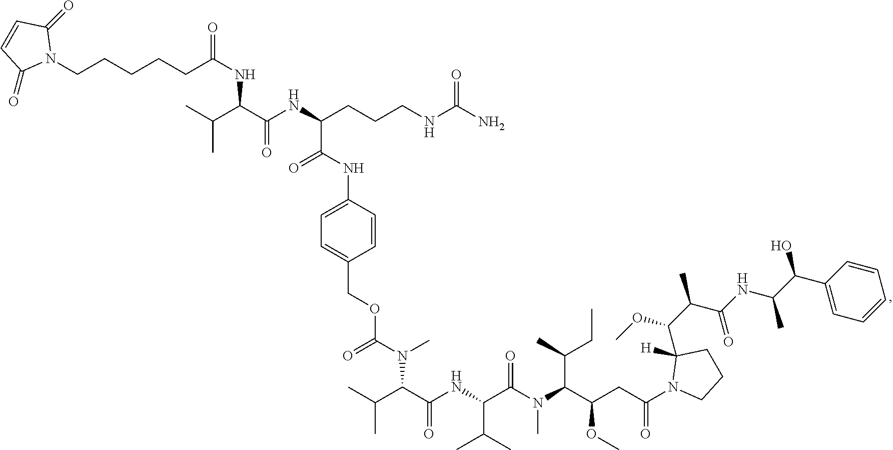 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

or a pharmaceutically acceptable salt thereof.

In some embodiments, the CEACAM5-ADC that comprises an anti-CEACAM5 antibody conjugated to a vcMMAE (valine-citruline-monomethyl auristatin E), the anti-CEACAM5 antibody comprises a heavy chain comprising the amino acid sequence of SEQ ID NO: 22 or 33 (with or without the C-terminal lysine, respectively), and a light chain comprising the amino acid sequence of SEQ ID NO: 23, and wherein the vcMMAE comprises the structure:

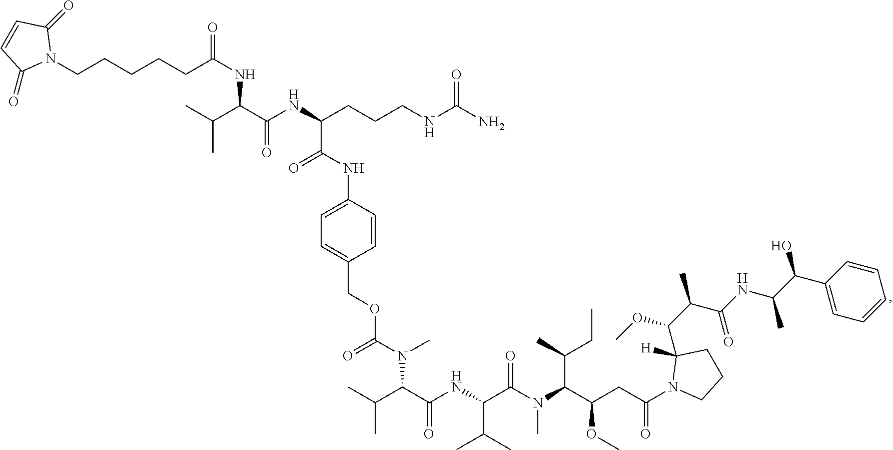 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

or a pharmaceutically acceptable salt thereof.

Also provided herein is a CEACAM5-ADC that comprises an anti-CEACAM5 antibody conjugated to a vcMMAE (valine-citruline-monomethyl auristatin E), wherein the anti-CEACAM5 antibody comprises a heavy chain comprising the amino acid sequence of SEQ ID NO: 22 or 33 (with or without the C-terminal lysine, respectively), and a light chain comprising the amino acid sequence of SEQ ID NO: 19, and wherein the vcMMAE comprises the structure:

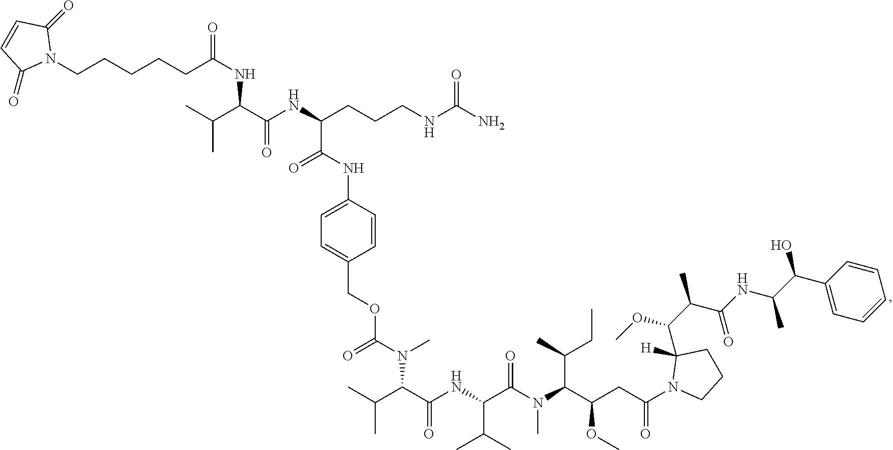 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

or a pharmaceutically acceptable salt thereof. In some embodiments, the CEACAM5 antibody-drug conjugate (CEACAM5-ADC) comprises the structure:

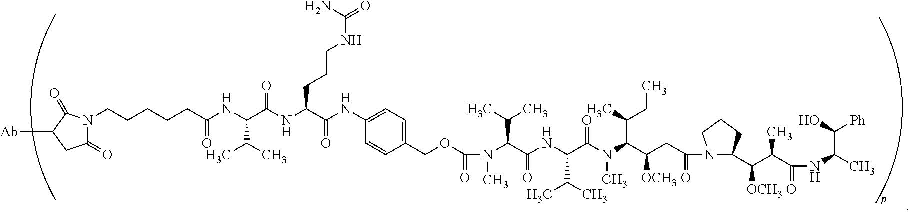

In some embodiments, the CEACAM5-ADC comprises the structure:

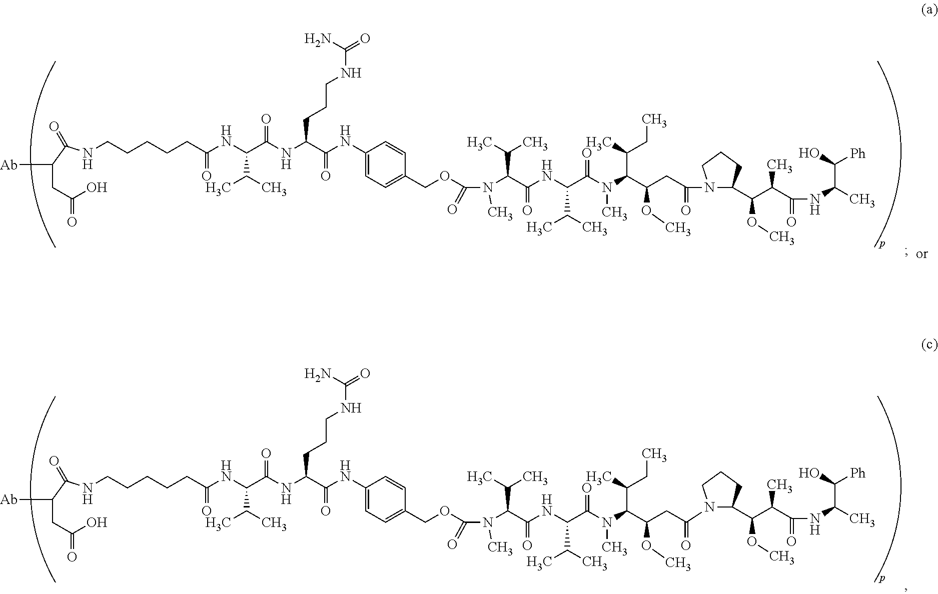

In some embodiments, p is about 1 to about 8. In some embodiments, p is about 1 to about 4. In yet other embodiments, p is about 4.

In some embodiments of the CEACAM5-ADC disclosed herein, the vcMMAE to antibody ratio is from about 1 to about 8. In some embodiments, the vcMMAE to antibody ratio is about 1 to about 4. In some embodiments, the vcMMAE to antibody ratio is about 4.

Also provided is a pharmaceutical composition comprising a therapeutically effective amount of a CEACAM5-ADC disclosed herein, and a pharmaceutically acceptable carrier. In some embodiments, a pharmaceutical composition for the treatment of cancer, comprising an a CEACAM5-ADC disclosed herein is provided. In some embodiments, an anti-cancer agent comprises an a CEACAM5-ADC disclosed here. In some embodiments, use of a CEACAM5-ADC disclosed herein can be used to treat cancer or in the manufacture of a medicament for the treatment of cancer.

In some embodiments, a method of treating a cancer in a subject is provided, wherein the method comprises administering to the subject in need thereof a therapeutically effective amount of a pharmaceutical composition or a CEACAM5-ADC disclosed herein. In some embodiments, a CEACAM5-ADC disclosed herein is for use as a medicament. In some embodiments, a CEACAM5-ADC disclosed herein is for the treatment of cancer.

### BRIEF DESCRIPTION OF THE DRAWINGS

FIG. 1 is a saturation curve depicting binding of CEACAM5 specific antibodies mAb2 and SG-105.2E4 to NCI-H1395 cells as measured by indirect flowcytometry.

FIG. 2A-2B shows the internalization rate for CEACAM5 binding antibodies and corresponding controls for HPAFII cells (FIG. 2A) and NCI-H2122 cells (FIG. 2B).

FIG. 3 shows competition binding of unlabeled mAb1 with Alexa Fluor 488-labelled SG.105.2E4 antibody. Unlabeled SG-105.2E4 antibody were used as a positive control.

FIG. 4 shows the results of an ADCC mediated assay with the SG105.2E4 EC antibody, SG-105.2E4 EC4-1006 ADC, and controls.

FIG. 5 shows the results of an ADCP mediated assay with the SG-105.2E4 SC4 antibody, SG-105.2E4 EC4-1006 ADC, and controls.

FIG. 6A-6B shows the results of a cell viability assay for CEACAM5-expressing cell line HPAII (FIG. 6A) and NCI-H2122 (FIG. 6B) when cells were treated with SG-105.2E4 EC4-1006 ADC, as compared to controls and mAb2. CEACAM5 antibody binding as quantitated by QFACS (specific binding sites/cell) is specified in parentheses.

FIG. 7A-7B shows the anti-tumor activity of SG-105.2E4 EC4-1006 ADC in HPAFII and (pancreatic) (FIG. 7A) and NCI-H2122 (NSCLC) (FIG. 7B) xenograft models.

FIG. 8A-8C shows the anti-tumor activity of SG-105.2E4 EC4-1006 ADC in NSCLC patient (CTG-0838 (FIG. 8A), CTG-0860 (FIG. 8B), and CTG-0828 (FIG. 8C)) patient-derived xenograft (PDX) models.

FIG. 9A-9B shows the systemic exposure to (FIG. 9A) gTAb and (FIG. 9B) acMMAE of SGD-105.2E4 EC4-1006 ADC compared to mAb1 EC4-1006 ADC.

### DETAILED DESCRIPTION

The present invention may be understood more readily by reference to the following detailed description of the embodiments of the invention and the Examples included herein. It is to be understood that this invention is not limited to specific methods of making that may of course vary. It is to be also understood that the terminology used herein is for the purpose of describing specific embodiments only and is not intended to be limiting.

Exemplary embodiments (E) of the invention provided herein include:
E1. An isolated antibody that binds to human CEACAM5 and comprises a heavy chain variable region (VH) and a light chain variable region (VL), wherein:
   a) the VH CDR1 comprises the amino acid sequence of SEQ ID NO: 8,
   b) the VH CDR2 comprises the amino acid sequence of SEQ ID NO: 9,
   c) the VH CDR3 comprises the amino acid sequence of SEQ ID NO: 10,
   d) the VL CDR1 comprises the amino acid sequence of SEQ ID NO: 11,
   e) the VL CDR2 comprises the amino acid sequence of SEQ ID NO: 12, and
   f) the VL CDR3 comprises the amino acid sequence of SEQ ID NO: 13.
E2. The antibody of E1, wherein the VH comprises the amino acid sequence of SEQ ID NO: 14 or a variant of SEQ ID NO: 14 comprising one to four amino acid substitutions at residues that are not within a CDR, and the VL comprises the amino acid sequence of SEQ ID NO: 15 or a variant of SEQ ID NO: 15 thereof comprising one to four amino acid substitutions at residues that are not within a CDR.
E3. The antibody of E3, wherein the VH has at least 95% sequence identity to the amino acid sequence of SEQ ID NO: 14, and the VL has at least 95% sequence identity to the amino acid sequence of SEQ ID NO: 15.
E4. The antibody of E3, wherein the VH comprises the amino acid sequence of SEQ ID NO: 14, and the VL comprises the amino acid sequence of SEQ ID NO: 15.
E5. An isolated antibody that binds to human CEACAM5 and comprises a heavy chain variable region (VH) and a light chain variable region (VL), wherein:
   a) the VH CDR1, VH CDR2, and VH CDR3 comprises the VH CDR1, VH CDR2, and VH CDR3 amino acid sequences, respectively, of SEQ ID NO: 14; and
   b) the VL CDR1, VL CDR2, and VL CDR3 comprises the VL CDR1, VL CDR2, and VL CDR3 amino acid sequences, respectively, of SEQ ID NO: 15, wherein the CDRs are the Kabat defined CDRs, the Chothia-defined CDRs, or the AbM-defined CDRs.
E6. The antibody of any ones E1-5, wherein the antibody further comprises a heavy chain constant region.
E7. The antibody of E6, wherein the heavy chain constant region comprises the amino acid sequence of SEQ ID NO: 16 or SEQ ID NO: 20.
E8. The antibody of E7, wherein the C-terminal lysine of the heavy chain has been removed.
E9. The antibody of any one of E1-8, wherein the antibody further comprises a light chain constant region.
E10. The antibody of E9, wherein the light chain constant region comprises the amino acid sequence of SEQ ID NO: 17 or SEQ ID NO: 21.
E11. An isolated antibody that binds to human CEACAM5 comprising: a heavy chain comprising the amino acid sequence of SEQ ID NO: 18, and a light chain comprising the amino acid sequence of SEQ ID NO: 19.
E12. An isolated antibody that binds to human CEACAM5 comprising: a heavy chain comprising the amino acid sequence of SEQ ID NO: 31, and a light chain comprising the amino acid sequence of SEQ ID NO: 19.
E13. An isolated antibody that binds to human CEACAM5 comprising: a heavy chain comprising the amino acid sequence of SEQ ID NO: 18, and a light chain comprising the amino acid sequence of SEQ ID NO: 23.
E14. An isolated antibody that binds to human CEACAM5 comprising: a heavy chain comprising the amino acid sequence of SEQ ID NO: 31, and a light chain comprising the amino acid sequence of SEQ ID NO: 23.
E15. An isolated antibody that binds to human CEACAM5 comprising: a heavy chain comprising the amino acid sequence of SEQ ID NO: 22, and a light chain comprising the amino acid sequence of SEQ ID NO: 23.
E16. An isolated antibody that binds to human CEACAM5 comprising: a heavy chain comprising the amino acid sequence of SEQ ID NO: 33, and a light chain comprising the amino acid sequence of SEQ ID NO: 23.
E17. An isolated antibody that binds to human CEACAM5 comprising: a heavy chain comprising the amino acid sequence of SEQ ID NO: 22, and a light chain comprising the amino acid sequence of SEQ ID NO: 19.
E18. An isolated antibody that binds to human CEACAM5 comprising: a heavy chain comprising the amino acid sequence of SEQ ID NO: 33, and a light chain comprising the amino acid sequence of SEQ ID NO: 19.
E19. The antibody of any one of E1-18, wherein the antibody does not bind to cynomolgus monkey CEACAM5.
E20. The antibody of any one of E1-19, wherein the antibody does not bind the A2-B2 domains of human CEACAM5.
E21. The antibody of any one of E1-20, wherein the antibody does not bind the A3-B3 domains of human CEACAM5.
E22. The antibody of any one of E1-21, wherein the antibody binds the N, A1, A2 or N-A1-A2 domains of human CEACAM5.
E23. An isolated polynucleotide encoding the antibody of any one of E1-22.
E24. A pharmaceutical composition comprising a therapeutically effective amount of the antibody of any one of E1-22 and a pharmaceutically acceptable carrier.
E25. A method of treating a cancer in a subject, the method comprising administering to the subject in need thereof a therapeutically effective amount of the pharmaceutical composition of E24 or the antibody of any one of E1-22.
E26. A CEACAM5 antibody-drug conjugate (CEACAM5-ADC), wherein the CEACAM5-ADC comprises an anti-CEACAM5 antibody conjugated to a vcMMAE (valine-citruline-monomethyl auristatin E), wherein the anti-CEACAM5 antibody comprises a heavy chain variable region (VH) and a light chain variable region (VL), wherein the VH-CDR1, VH-CDR2, VH-CDR3 and VL-CDR1, VL-CDR2, and VL-CDR3 sequences are SEQ ID NOs: 8-13, respectively;
   wherein the vcMMAE comprises the structure:

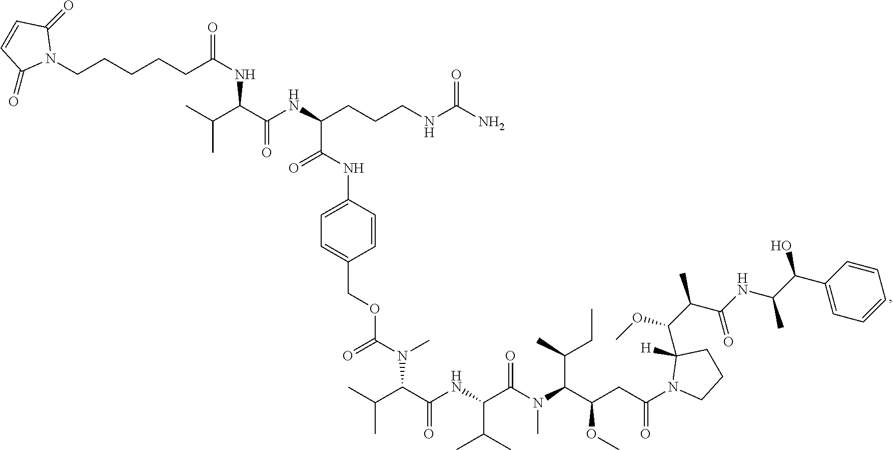 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

or a pharmaceutically acceptable salt thereof.
E27. The CEACAM5-ADC of E26, wherein the VH comprises the amino acid sequence of SEQ ID NO: 14 or a variant of SEQ ID NO: 14 comprising one to four amino acid substitutions at residues that are not within a CDR, and the VL comprises the amino acid sequence of SEQ ID NO: 15 or a variant of SEQ ID NO: 15 thereof comprising one to four amino acid substitutions at residues that are not within a CDR.
E28. The CEACAM5-ADC of E26, wherein the VH has at least 95% sequence identity to the amino acid sequence of SEQ ID NO: 14, and the VL has at least 95% sequence identity to the amino acid sequence of SEQ ID NO: 15.
E29. The CEACAM5-ADC of E26, wherein the VH comprises the amino acid sequence of SEQ ID NO: 14, and the VL comprises the amino acid sequence of SEQ ID NO: 15.
E30. A CEACAM5 antibody-drug conjugate (CEACAM5-ADC), wherein the CEACAM5-ADC comprises an anti-CEACAM5 antibody conjugated to a vcMMAE (valine-citruline-monomethyl auristatin E), wherein the anti-CEACAM5 antibody comprises a heavy chain variable region (VH) and a light chain variable region (VL), wherein (a) the VH CDR1, VH CDR2, and VH CDR3 comprises the VH CDR1, VH CDR2, and VH CDR3 amino acid sequences, respectively, of SEQ ID NO: 14; and (b) the VL CDR1, VL CDR2, and VL CDR3 comprises the VL CDR1, VL CDR2, and VL CDR3 amino acid sequences, respectively, of SEQ ID NO: 15;
wherein the CDRs are the Kabat defined CDRs, the Chothia-defined CDRs, or the AbM-defined CDRs; and
wherein the vcMMAE comprises the structure:

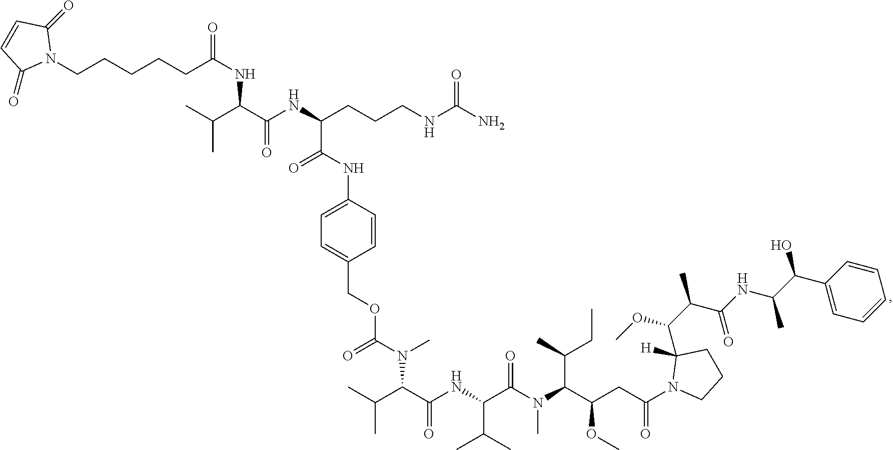 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

or a pharmaceutically acceptable salt thereof.
E31. The CEACAM5-ADC of any ones of E26-30, wherein the antibody further comprises a heavy chain constant region.
E32. The CEACAM5-ADC of E31, wherein the heavy chain constant region comprises the amino acid sequence of SEQ ID NO: 16 or SEQ ID NO: 20.
E33. The CEACAM5-ADC of E32, wherein the C-terminal lysine of the heavy chain has been removed.
E34. The CEACAM5-ADC of any one of E26-33, wherein the antibody further comprises a light chain constant region.
E35. The CEACAM5-ADC of E34, wherein the light chain constant region comprises the amino acid sequence of SEQ ID NO: 17 or SEQ ID NO: 21.
E36. A CEACAM5 antibody-drug conjugate (CEACAM5-ADC), wherein the CEACAM5-ADC comprises an anti-CEACAM5 antibody conjugated to a vcMMAE (valine-citruline-monomethyl auristatin E), wherein the anti-CEACAM5 antibody comprises a heavy chain comprising the amino acid sequence of SEQ ID NO: 18, and a light chain comprising the amino acid sequence of SEQ ID NO: 19, and wherein the vcMMAE comprises the structure:

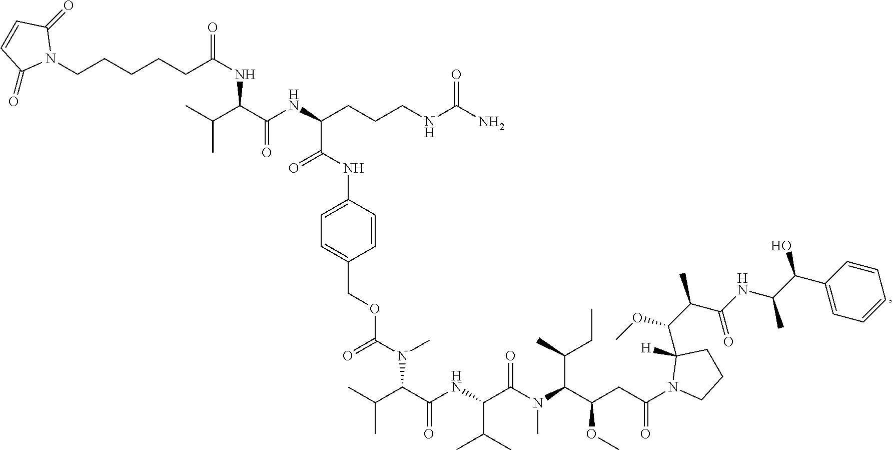 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

or a pharmaceutically acceptable salt thereof.
E37. A CEACAM5 antibody-drug conjugate (CEACAM5-ADC), wherein the CEACAM5-ADC comprises an anti-CEACAM5 antibody conjugated to a vcMMAE (valine-citruline-monomethyl auristatin E), wherein the anti-CEACAM5 antibody comprises a heavy chain comprising the amino acid sequence of SEQ ID NO: 31, and a light chain comprising the amino acid sequence of SEQ ID NO: 19, and wherein the vcMMAE comprises the structure:

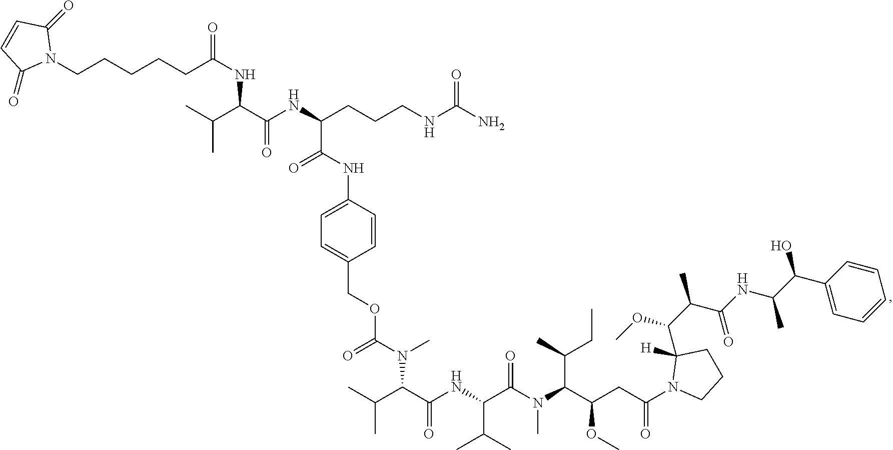 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

or a pharmaceutically acceptable salt thereof.
E38. A CEACAM5 antibody-drug conjugate (CEACAM5-ADC), wherein the CEACAM5-ADC comprises an anti-CEACAM5 antibody conjugated to a vcMMAE (valine-citruline-monomethyl auristatin E), wherein the anti-CEACAM5 antibody comprises a heavy chain comprising the amino acid sequence of SEQ ID NO: 18, and a light chain comprising the amino acid sequence of SEQ ID NO: 23, and wherein the vcMMAE comprises the structure:

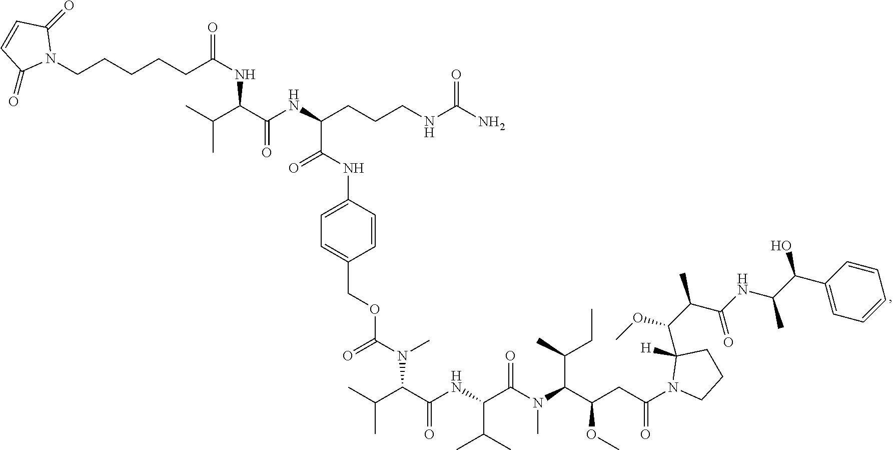 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

or a pharmaceutically acceptable salt thereof.
E39. A CEACAM5 antibody-drug conjugate (CEACAM5-ADC), wherein the CEACAM5-ADC comprises an anti-CEACAM5 antibody conjugated to a vcMMAE (valine-citruline-monomethyl auristatin E), wherein the anti-CEACAM5 antibody comprises a heavy chain comprising the amino acid sequence of SEQ ID NO: 31, and a light chain comprising the amino acid sequence of SEQ ID NO: 23, and wherein the vcMMAE comprises the structure:

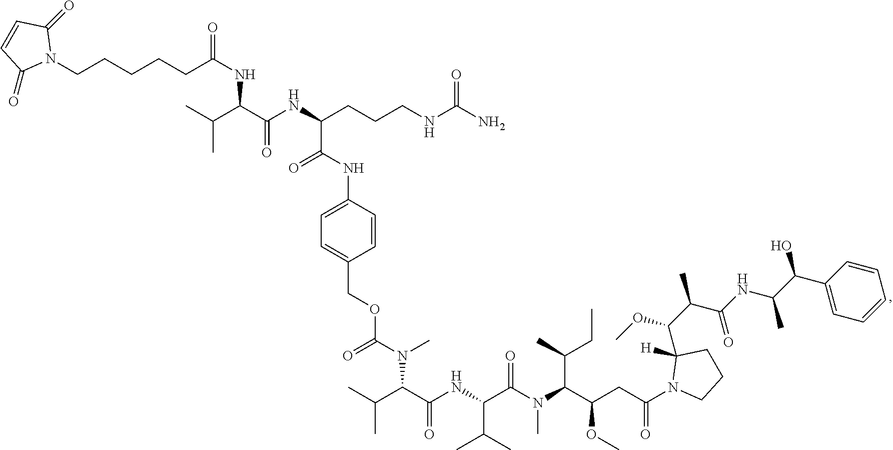 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

or a pharmaceutically acceptable salt thereof.
E40. A CEACAM5 antibody-drug conjugate (CEACAM5-ADC), wherein the CEACAM5-ADC comprises an anti-CEACAM5 antibody conjugated to a vcMMAE (valine-citruline-monomethyl auristatin E), wherein the anti-CEACAM5 antibody comprises a heavy chain comprising the amino acid sequence of SEQ ID NO: 22, and a light chain comprising the amino acid sequence of SEQ ID NO: 23, and wherein the vcMMAE comprises the structure:

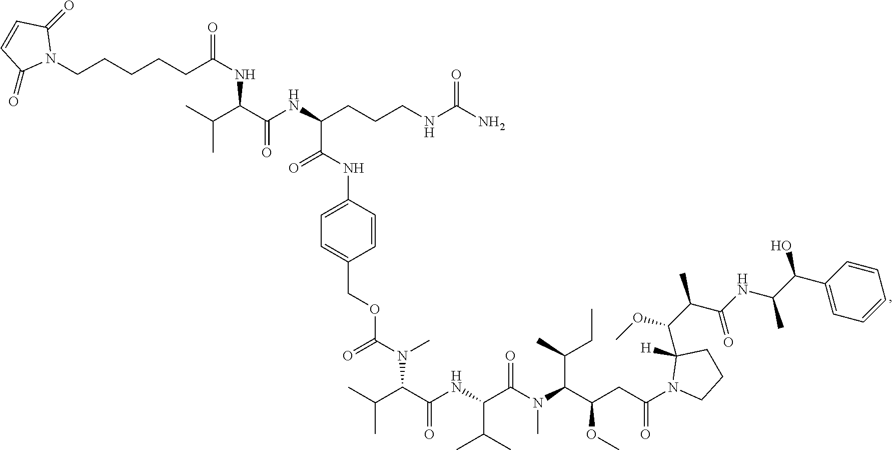 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

or a pharmaceutically acceptable salt thereof.
E41. A CEACAM5 antibody-drug conjugate (CEACAM5-ADC), wherein the CEACAM5-ADC comprises an anti-CEACAM5 antibody conjugated to a vcMMAE (valine-citruline-monomethyl auristatin E), wherein the anti-CEACAM5 antibody comprises a heavy chain comprising the amino acid sequence of SEQ ID NO: 33, and a light chain comprising the amino acid sequence of SEQ ID NO: 23, and wherein the vcMMAE comprises the structure:

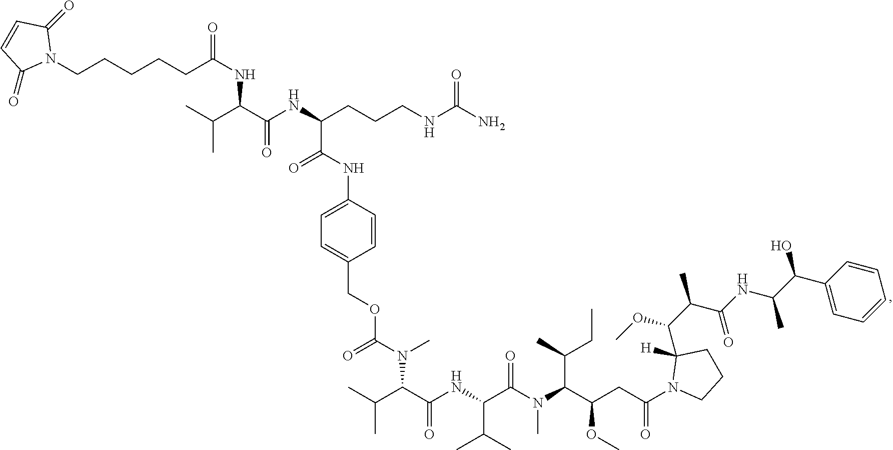 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

or a pharmaceutically acceptable salt thereof.
E42. A CEACAM5 antibody-drug conjugate (CEACAM5-ADC), wherein the CEACAM5-ADC comprises an anti-CEACAM5 antibody conjugated to a vcMMAE (valine-citruline-monomethyl auristatin E), wherein the anti-CEACAM5 antibody comprises a heavy chain comprising the amino acid sequence of SEQ ID NO: 22, and a light chain comprising the amino acid sequence of SEQ ID NO: 19, and wherein the vcMMAE comprises the structure:

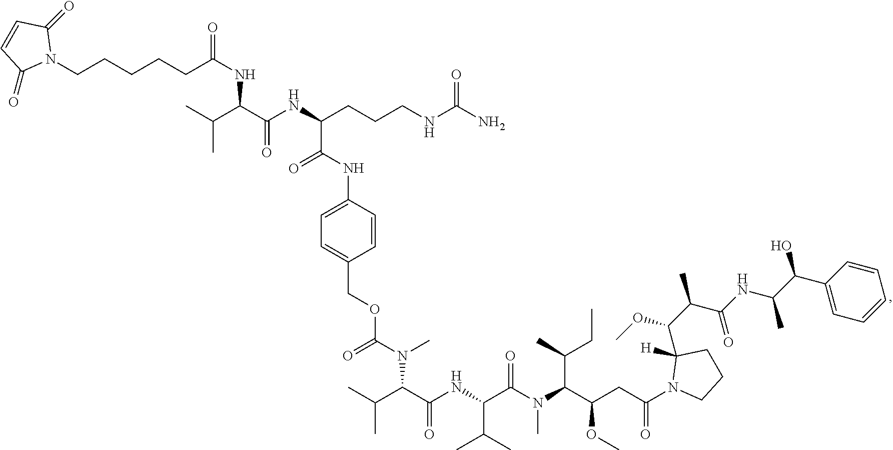 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

or a pharmaceutically acceptable salt thereof.
E43. A CEACAM5 antibody-drug conjugate (CEACAM5-ADC), wherein the CEACAM5-ADC comprises an anti-CEACAM5 antibody conjugated to a vcMMAE (valine-citruline-monomethyl auristatin E), wherein the anti-CEACAM5 antibody comprises a heavy chain comprising the amino acid sequence of SEQ ID NO: 33, and a light chain comprising the amino acid sequence of SEQ ID NO: 19, and wherein the vcMMAE comprises the structure:

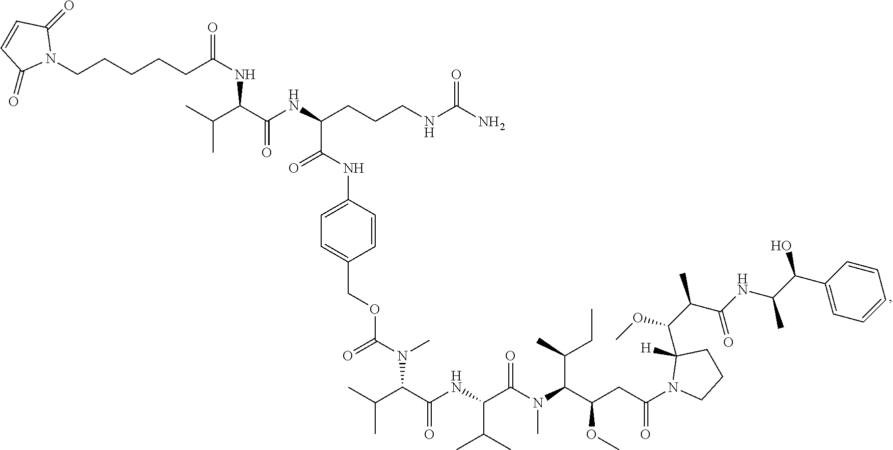 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

or a pharmaceutically acceptable salt thereof.
E44. The CEACAM5 antibody-drug conjugate (CEACAM5-ADC) of any one of claims 26-43, wherein the CEACAM5-ADC comprises the structure:

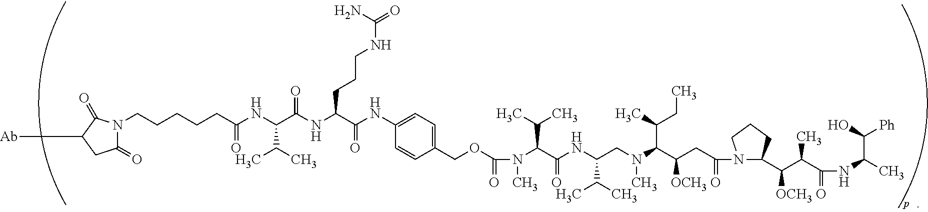

E45. The CEACAM5 antibody-drug conjugate (CEACAM5-ADC) of any one of claims 26-43, wherein the CEACAM5-ADC comprises the structure:

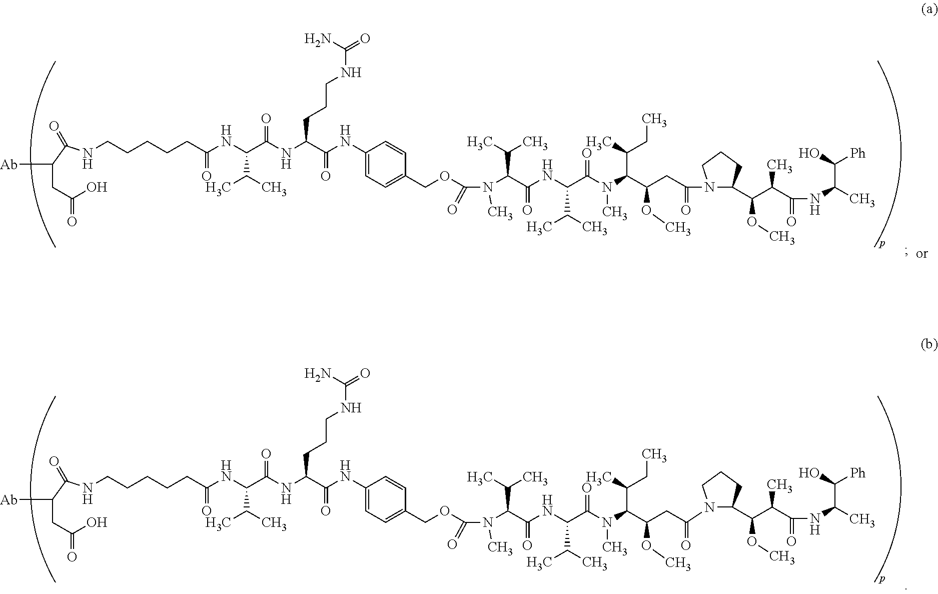

E46. The CEACAM5-ADC of any one of E36-43, wherein a vcMMAE to antibody ratio is from about 1 to about 8 or about 1 to about 4.
E47. The CEACAM5-ADC of any one of E36-43, wherein the vcMMAE to antibody ratio is about 4.
E48. The CEACAM5-ADC of E44 or E45, wherein p is about 1 to about 8, or about 1 to about 4.
E49. The CEACAM5-ADC of E44 or E45, wherein p is about 4.
E50. A pharmaceutical composition comprising a therapeutically effective amount of the CEACAM5-ADC of any one of E26-49, and a pharmaceutically acceptable carrier.
E51. A method of treating a cancer in a subject, the method comprising administering to the subject in need thereof a therapeutically effective amount of the CEACAM5-ADC of any one of claims 26-49 or the pharmaceutical composition of E50.

The section headings used herein are for organizational purposes only and are not to be construed as limiting the subject matter described.

All references cited herein, including patent applications, patent publications, UniProtKB accession numbers are herein incorporated by reference, as if each individual reference were specifically and individually indicated to be incorporated by reference in its entirety.

The techniques and procedures described or referenced herein are generally well understood and commonly employed using conventional methodology by those skilled in the art, such as, for example, the widely utilized methodologies described in Sambrook et al, Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds); Gene Transfer Vectors for Mammalian Cells (J. M. Miller and M. P. Calos, eds., 1987); PCR: The Polymerase Chain Reaction, (Mullis et al, eds., 1994); Current Protocols in Immunology (J. E. Coligan et al, eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999)); The Antibodies (M. Zanetti and J. D. Capra, eds., Harwood Academic Publishers, 1995); and updated versions thereof.

### I. DEFINITIONS

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

An “antibody-drug conjugate” or “ADC” refers to an antibody conjugated to a cytotoxic agent or cytostatic agent. Typically, antibody-drug conjugates bind to a target antigen (e.g., CEACAM5) on a cell surface, followed by internalization of the antibody-drug conjugate into the cell and subsequent release of the drug into the cell. In certain exemplary embodiments, an antibody-drug conjugate is a CEACAM5-ADC.

A “polypeptide” or “protein” (used interchangeably herein) refers to a chain of amino acids of any length. The chain may be linear or branched. The chain may comprise one or more of modified amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

The terms “amino-terminal” and “carboxy-terminal” denote positions within polypeptides. Where the context allows, these terms are used with reference to a particular sequence or portion of a polypeptide to denote proximity or relative position. For example, a certain sequence positioned carboxy-terminal to a reference sequence within a polypeptide is located proximal to the carboxy terminus of the reference sequence, but is not necessarily at the carboxy terminus of the complete polypeptide.

For purposes of classifying amino acids substitutions as conservative or nonconservative, a “conservative substitution” refers to replacement of one amino acid by a biologically, chemically or structurally similar residue. Biologically similar means that the substitution does not destroy a biological activity. Structurally similar means that the amino acids have side chains with similar length, such as alanine, glycine and serine or a similar size. Chemical similarity means that the residues have the same charge or are both hydrophilic or hydrophobic. Particular examples include the substitution of a hydrophobic residue, such as isoleucine, valine, leucine or methionine with another, or the substitution of one polar residue for another, such as the substitution of arginine for lysine, glutamic acid for aspartic acid or glutamine for asparagine, serine for threonine, and the like. Particular examples of conservative substitutions include the substitution of a hydrophobic residue such as isoleucine, valine, leucine or methionine for one another, the substitution of a polar residue for another, such as the substitution of arginine for lysine, glutamic acid for aspartic acid, or glutamine for asparagine, and the like. Conservative amino acid substitutions typically include, for example, substitutions within the following groups: glycine, alanine, valine, isoleucine, leucine; aspartic acid, glutamic acid; asparagine, glutamine; serine, threonine; lysine, arginine; and phenylalanine, tyrosine.

A “polynucleotide” or “nucleic acid,” (used interchangeably herein) refers to a chain of nucleotides of any length, and includes DNA and RNA. The nucleotides can be deoxyribonucleotides, ribonucleotides, modified nucleotides or bases or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain.

The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses, pyranose sugars, furanose sugars, sedoheptuloses, acyclic analogs and abasic nucleoside analogs such as methyl riboside.

Two sequences (e.g., polypeptide sequences or nucleotide sequences) have “100% sequence identity” if the residues (e.g., amino acid or nucleic acid) of the two sequences are the same when aligned for maximal correspondence. The term “identity” or “identical to” refers to the overall relatedness between polymeric molecules, e.g., between nucleic acid molecules (e.g., DNA molecules or RNA molecules) or between polypeptide molecules. “Identity” measures the percent of identical matches between two or more sequences with gap alignments addressed by a particular mathematical model of computer programs (e.g., algorithms), which are well known in the art. Calculation of the percent identity of two nucleic acid or polypeptide sequences, for example, can be performed by aligning the two sequences for optimal comparison purposes (e.g., gaps can be introduced in one or both of a first and a second sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of a reference sequence. The nucleotides or amino acids at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm.

To determine percent identity, sequences can be aligned using the methods and computer programs, including BLAST, available over the world wide web at ncbi.nlm.nih.gov/BLAST/. Other alignment programs include MegAlign® program in the Lasergene® suite of bioinformatics software (DNASTAR®, Inc., Madison, WI). Another alignment algorithm is FASTA, available in the Genetics Computing Group (GCG) package, from Madison, Wis., USA. Other techniques for alignment are described in Methods in Enzymology, vol. 266: Computer Methods for Macromolecular Sequence Analysis (1996), ed. Doolittle, Academic Press, Inc. Of particular interest are alignment programs that permit gaps in the sequence. Smith-Waterman is one type of algorithm that permits gaps in sequence alignments. See Meth. Mal. Biol. 70:173-187 (1997). Also, the GAP program using the Needleman and Wunsch alignment method can be utilized to align sequences. See J. Mal. Biol. 48:443-453 (1970).

Also, of interest is the BestFit program using the local homology algorithm of Smith and Waterman (1981, Advances in Applied Mathematics 2:482-489) to determine sequence identity. The gap generation penalty will generally range from 1 to 5, usually 2 to 4 and in some embodiments will be 3. The gap extension penalty will generally range from about 0.01 to 0.20 and in some instances will be 0.10. The program has default parameters determined by the sequences inputted to be compared. Preferably, the sequence identity is determined using the default parameters determined by the program. This program is available also from Genetics Computing Group (GCG) package, from Madison, WI, USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters: Mismatch Penalty: 1.00; Gap Penalty: 1.00; Gap Size Penalty: 0.33; and Joining Penalty: 30.0.

Percentage sequence identities are determined with antibody sequences maximally aligned by the Kabat numbering convention. After alignment, if a subject antibody region (e.g., the entire variable domain of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

Compositions or methods “comprising” one or more recited elements may include other elements not specifically recited. For example, a composition that comprises antibody may contain the antibody alone or in combination with other ingredients.

Designation of a range of values includes all integers within or defining the range.

In antibodies or other proteins described herein, reference to amino acid residues corresponding to those specified by SEQ ID NO includes post-translational modifications of such residues.

The term “antibody” refers to an immunoglobulin molecule capable of specific binding to a target, such as a polypeptide, carbohydrate, polynucleotide, lipid, etc., through at least one antigen binding site, located in the variable region of the immunoglobulin molecule. As used herein, the term “antibody” can encompass any type of antibody (e.g., monospecific, bispecific), and includes portions of intact antibodies that retain the ability to bind to a given antigen (e.g., an “antigen-binding fragment”), and any other modified configuration of an immunoglobulin molecule that comprises an antigen binding site. An exemplary antibody comprises i) a variable region of the light chain, heavy chain or both and ii) a constant region of the heavy chain comprising three sequential immunoglobulin domains (CH1, CH2, and CH3) and of the light chain comprising a single immunoglobulin domain (CL).

An antibody includes an antibody of any class, such as IgG, IgA, or IgM (or sub-class thereof), and the antibody need not be of any particular class. Depending on the antibody amino acid sequence of the constant region of its heavy chains (HC), immunoglobulins can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁ and IgA₂. The heavy chain constant regions that correspond to the different classes of immunoglobulins are called alpha, delta, epsilon, gamma, and mu, respectively. The subunit structures and three-dimensional configurations of different classes of immunoglobulins are well known.

Examples of antibody antigen-binding fragments and modified configurations include (i) a Fab fragment (a monovalent fragment consisting of the VL, VH, CL and CH1 domains); (ii) a F(ab′)₂ fragment (a bivalent fragment comprising two Fab fragments linked by a disulfide bridge at the hinge region); and (iii) a Fv fragment consisting of the VL and VH domains of a single arm of an antibody. Furthermore, although the two domains of an Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)); see e.g., Bird et al., Science 1988; 242:423-426 and Huston et al., Proc. Natl. Acad. Sci. 1988 USA 85:5879-5883. Other forms of single chain antibodies, such as diabodies are also encompassed.

In addition, further encompassed are antibodies that are missing a C-terminal lysine (K) amino acid residue on a heavy chain polypeptide (e.g., human IgG1 heavy chain comprises a terminal lysine). As is known in the art, the C-terminal lysine is sometimes clipped during antibody production, resulting in an antibody with a heavy chain lacking the C-terminal lysine. Alternatively, an antibody heavy chain may be produced using a nucleic acid that does not include a C-terminal lysine.

The term antibody or antigen-binding fragment thereof includes a “conjugated” antibody or antigen-binding fragment thereof or an “antibody-drug conjugate (ADC)” in which an antibody or antigen-binding fragment thereof is covalently or non-covalently bound to a pharmaceutical agent, e.g., to a cytostatic or cytotoxic drug.

The term “genetically engineered antibodies” refers to an antibody in which the amino acid sequence has been varied from that of the native or parental antibody. The possible variations are many, and range from the changing of just one or a few amino acids to the complete redesign of, for example, the variable or constant region. Changes in the constant region are, in general, made to improve or alter characteristics such as, e.g., complement binding and other effector functions. Typically, changes in the variable region are made to improve antigen-binding characteristics, improve variable region stability, and/or reduce the risk of immunogenicity.

The term “chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

A “human antibody” refers to an antibody which possesses an amino acid sequence which corresponds to that of an antibody produced by a human or has been made using any technique for making fully human antibodies. For example, fully human antibodies may be obtained by using commercially available mice that have been engineered to express specific human immunoglobulin proteins, or by library (e.g., phage, yeast, or ribosome) display techniques for preparing fully human antibodies. This definition of a human antibody specifically excludes a humanized antibody comprising non-human antigen binding residues. This definition of a human antibody or antigen-binding fragment thereof includes intact or full-length antibodies and fragments thereof.

An “antigen-binding site of an antibody” is that portion of an antibody that is sufficient to bind to its antigen. The minimum such region is typically a variable domain or a genetically engineered variant thereof. Single domain binding sites can be generated from camelid antibodies (see Muyldermans and Lauwereys, Mol. Recog. 12:131-140, 1999; Nguyen et al., EMBO J. 19:921-930, 2000) or from VH domains of other species to produce single-domain antibodies (“dAbs,” see Ward et al., Nature 341:544-546, 1989; U.S. Pat. No. 6,248,516 to Winter et al). Commonly, an antigen-binding site of an antibody comprises both a heavy chain variable (VH) domain and a light chain variable (VL) domain that bind to a common epitope. Within the context of the present invention, an antibody may include one or more components in addition to an antigen-binding site, such as, for example, a second antigen-binding site of an antibody (which may bind to the same or a different epitope or to the same or a different antigen), a peptide linker, an immunoglobulin constant region, an immunoglobulin hinge, an amphipathic helix (see Pack and Pluckthun, Biochem. 31:1579-1584, 1992), a non-peptide linker, an oligonucleotide (see Chaudri et al., FEBS Letters 450:23-26, 1999), a cytostatic or cytotoxic drug, and the like, and may be a monomeric or multimeric protein. Examples of molecules comprising an antigen-binding site of an antibody are known in the art and include, for example, Fv, single-chain Fv (scFv), Fab, Fab′, F(ab′)₂, F(ab)c, diabodies, minibodies, nanobodies, Fab-scFv fusions, bispecific (scFv) 4-IgG, and bispecific (scFv) 2-Fab. (See, e.g., Hu et al, Cancer Res. 56:3055-3061, 1996; Atwell et al., Molecular Immunology 33:1301-1312, 1996; Carter and Merchant, Curr. Op. Biotechnol. 8:449-454, 1997; Zuo et al., Protein Engineering 13:361-367, 2000; and Lu et al., J. Immunol. Methods 267:213-226, 2002.)

The term “immunoglobulin” refers to a protein consisting of one or more polypeptides substantially encoded by immunoglobulin gene(s). One form of immunoglobulin constitutes the basic structural unit of native (i.e., natural or parental) antibodies in vertebrates. This form is a tetramer and consists of two identical pairs of immunoglobulin chains, each pair having one light chain and one heavy chain. In each pair, the light and heavy chain variable regions (VL and VH) are together primarily responsible for binding to an antigen, and the constant regions are primarily responsible for the antibody effector functions. Five classes of immunoglobulin protein (IgG, IgA, IgM, IgD, and IgE) have been identified in higher vertebrates. IgG comprises the major class, and it normally exists as the second most abundant protein found in plasma. In humans, IgG consists of four subclasses, designated IgG₁, IgG₂, IgG₃, and IgG₄.

Each immunoglobulin heavy chain possesses a constant region that consists of constant region protein domains (CH1, hinge, CH2, and CH3; IgG₃ also contains a CH4 domain) that are essentially invariant for a given subclass in a species. A “constant region” of an antibody refers to the constant region of the antibody light chain or the constant region of the antibody heavy chain, either alone or in combination. An IgG heavy chain constant region contains three sequential immunoglobulin domains (CH1, CH2, and CH3), with a hinge region between the CH1 and CH2 domains. An IgG light chain constant region contains a single immunoglobulin domain (CL).

DNA sequences encoding human and non-human immunoglobulin chains are known in the art. (See, e.g., Ellison et al., DNA 1:11-18, 1981; Ellison et al, Nucleic Acids Res. 10:4071-4079, 1982; Kenten et al., Proc. Natl. Acad. Set USA 79:6661-6665, 1982; Seno et al., Nucl. Acids Res. 11:719-726, 1983; Riechmann et al., Nature 332:323-327, 1988; Amster et al., Nucl. Acids Res. 8:2055-2065, 1980; Rusconi and Kohler, Nature 314:330-334, 1985; Boss et al., Nucl. Acids Res. 12:3791-3806, 1984; Bothwell et al., Nature 298:380-382, 1982; van der Loo et al., Immunogenetics 42:333-341, 1995; Karlin et al., J. Mol. Evol. 22:195-208, 1985; Kindsvogel et al., DNA 1:335-343, 1982; Breiner et al., Gene 18:165-174, 1982; Kondo et al., Eur. J. Immunol. 23:245-249, 1993; and GenBank Accession No. J00228.) For a review of immunoglobulin structure and function see Putnam, The Plasma Proteins, Vol V, Academic Press, Inc., 49-140, 1987; and Padlan, Mol. Immunol. 31:169-217, 1994. The term “immunoglobulin” is used herein for its common meaning, denoting an intact antibody, its component chains, or fragments of chains, depending on the context.

Full-length immunoglobulin “light chains” (about 25 kDa or 214 amino acids) are encoded by a variable region gene at the amino-terminus (encoding about 110 amino acids) and a by a kappa or lambda constant region gene at the carboxyl-terminus. Full-length immunoglobulin “heavy chains” (about 50 kDa or 446 amino acids) are encoded by a variable region gene (encoding about 116 amino acids) and a gamma, mu, alpha, delta, or epsilon constant region gene (encoding about 330 amino acids), the latter defining the antibody's isotype as IgG, IgM, IgA, IgD, or IgE, respectively. Within light and heavy chains, the variable and constant regions are joined by a “J” region of about 12 or more amino acids, with the heavy chain also including a “D” region of about 10 more amino acids. (See generally Fundamental Immunology (Paul, ed., Raven Press, N.Y., 2nd ed. 1989), Ch. 7).

An immunoglobulin “variable region” refers to the variable region of the antibody light chain or the variable region of the antibody heavy chain, either alone or in combination. As known in the art, the variable regions of the heavy and light chains (also referred to herein as a “light chain variable domain” (“VL domain”) or “heavy chain variable domain” (“VH domain”), respectively) each consist of four framework regions (FRs) connected by three complementarity determining regions (CDRs) also known as hypervariable regions, and contribute to the formation of the antigen binding site of antibodies. The framework regions serve to align the CDRs for specific binding to an epitope of an antigen. Thus, the term “CDR” refers to the amino acid residues of an antibody that are primarily responsible for antigen binding. From amino-terminus to carboxyl-terminus, both VL and VH domains comprise the following framework (FR) and CDR regions: FR1, CDR1, FR2, CDR2, FR3, CDR3, FR4. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonical class as the subject variable region (Chothia and Lesk, J Mol Biol 196 (4): 901-917, 1987).

In certain embodiments, definitive delineation of a CDR and identification of residues comprising the binding site of an antibody is accomplished by solving the structure of the antibody or solving the structure of the antibody-ligand complex. In certain embodiments, which can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the AbM definition, the contact definition, the extended definition, and the conformational definition.

The Kabat definition is a standard for numbering the residues in an antibody and is typically used to identify CDR regions. See, e.g., Johnson & Wu, 2000, Nucleic Acids Res., 28:214-8. The assignment of amino acids to each variable region domain of the antibodies disclosed herein is in accordance with the definitions of Kabat, Sequences of Proteins of Immunological Interest (National Institutes of Health, Bethesda, MD, 1987 and 1991). Kabat provides a widely used numbering convention (Kabat numbering) in which corresponding residues between different heavy chain variable regions or between different light chain variable regions are assigned the same number. CDRs 1, 2 and 3 of a VL domain are also referred to herein, respectively, as VL CDR1, VL CDR2, VL CDR3, or alternatively, as CDR-L1, CDR-L2 and CDR-L3. CDRs 1, 2 and 3 of a VH domain are also referred to herein, respectively, as VH CDR1, VH CDR2, and VH CDR3, or alternatively, as CDR-H1, CDR-H2 and CDR-H3. If so noted, the assignment of CDRs can be in accordance with IMGT® (Lefranc et al., Developmental & Comparative Immunology 27:55-77; 2003) in lieu of Kabat. Numbering of the heavy and light chain constant region is via the EU index as set forth in Kabat (Kabat, Sequences of Proteins of Immunological Interest, National Institutes of Health, Bethesda, MD, 1987 and 1991).

The Chothia definition is similar to the Kabat definition, but the Chothia definition takes into account positions of certain structural loop regions. See, e.g., Chothia et al., 1986, J. Mol. Biol., 196:901-17; Chothia et al., 1989, Nature, 342:877-83. The extended definition is the combination of the Kabat and Chothia definitions. The AbM definition uses an integrated suite of computer programs produced by Oxford Molecular Group that model antibody structure. See, e.g., Martin et al., 1989, Proc Natl Acad Sci (USA), 86:9268-9272; “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999, “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any one or more of Kabat, Chothia, extended, AbM, contact, or conformational definitions.

A “Fc domain” refers to the portion of an immunoglobulin (Ig) molecule that correlates to a crystallizable fragment obtained by papain digestion of an Ig molecule. As used herein, the term relates to the 2-chained constant region of an antibody, each chain excluding the first constant region immunoglobulin domain. Within an Fc domain, there are two “Fc chains” (e.g., a “first Fc chain” and a “second Fc chain”). “Fc chain” generally refers to the C-terminal portion of an antibody heavy chain. Thus, Fc chain refers to the last two constant region immunoglobulin domains (CH2 and CH3) of IgA, IgD, and IgG heavy chains, and the last three constant region immunoglobulin domains of IgE and IgM heavy chains, and optionally the flexible hinge N-terminal to these domains.

Although the boundaries of the Fc chain may vary, the human IgG heavy chain Fc chain is usually defined to comprise residues C226 or P230 to its carboxyl-terminus, wherein the numbering is according to the EU index of Edelman et al., Proc. Natl. Acad. Sci. USA 1969; 63 (1): 78-85 and as described in Kabat et al., 1991. Typically, the Fc chain comprises from about amino acid residue 236 to about 447 of the human IgG₁ heavy chain constant region. “Fc chain” may refer to this polypeptide in isolation, or in the context of a larger molecule (e.g., in an antibody heavy chain or Fc fusion protein).

A “functional” Fc domain refers to an Fc domain that possesses at least one effector function of a native sequence Fc domain. Exemplary “effector functions” include C1q binding; complement dependent cytotoxicity (CDC); Fc receptor binding; antibody-dependent cell-mediated cytotoxicity (ADCC); phagocytosis; down-regulation of cell surface receptors (e.g., B cell receptor); and B cell activation, etc. Such effector functions generally require the Fc domain to be combined with a binding domain (e.g., an antibody variable region) and can be assessed using various assays known in the art for evaluating such antibody effector functions.

A “native sequence” Fc chain refers to a Fc chain that comprises an amino acid sequence identical to the amino acid sequence of an Fc chain found in nature. A “variant” Fc chain comprises an amino acid sequence which differs from that of a native sequence Fc chain by virtue of at least one amino acid modification.

Unless the context dictates otherwise, the term “monoclonal antibody” is not limited to antibodies produced through hybridoma technology. The term “monoclonal antibody” (mAb) can include an antibody that is derived from a single clone, including any eukaryotic, prokaryotic or phage clone. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature 256:495, or may be made by recombinant DNA methods such as described in U.S. Pat. No. 4,816,567. In another example, monoclonal antibodies may be isolated from phage libraries such as those generated using the techniques described in McCafferty et al., 1990, Nature 348:552-554. In particular embodiments, the antibodies described herein are monoclonal antibodies. In some embodiments, an antibody may be a “monospecific antibody,” which refers to an antibody that comprises one or more antigen binding sites per molecule such that any and all binding sites of the antibody specifically recognize the identical epitope on the antigen. Thus, in cases where a monospecific antibody has more than one antigen binding site, the binding sites compete with each other for binding to one antigen molecule.

The term “humanized VH domain” or “humanized VL domain” refers to an immunoglobulin VH or VL domain comprising some or all CDRs entirely or substantially from a non-human donor immunoglobulin (e.g., a mouse or rat) and variable domain framework sequences entirely or substantially from human immunoglobulin sequences. The non-human immunoglobulin providing the CDRs is called the “donor” and the human immunoglobulin providing the framework is called the “acceptor.” In some instances, humanized antibodies will retain some non-human residues within the human variable domain framework regions to enhance proper binding characteristics (e.g., mutations in the frameworks may be required to preserve binding affinity when an antibody is humanized).

A “humanized” antibody refers to a non-human (e.g., murine) antibody that is a chimeric antibody that contains minimal sequence derived from non-human immunoglobulin. Preferably, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. The humanized antibody may comprise residues that are found neither in the recipient antibody nor in the imported CDR or framework sequences, but are included to further refine and optimize antibody performance.

Antibodies are typically provided in isolated form. An “isolated” molecule (e.g. antibody) refers to a molecule that by virtue of its origin or source of derivation (1) is not associated with naturally associated components that accompany it in its native state, (2) is substantially free of other molecules from the same source, e.g., species, cell from which it is expressed, library, etc., (3) is expressed by a cell from a different species, or (4) does not occur in nature. Thus, a molecule that is chemically synthesized, or expressed in a cellular system different from the system from which it naturally originates, will be “isolated” from its naturally associated components. A molecule also may be rendered substantially free of naturally associated components by isolation, using purification techniques well known in the art. An isolated antibody means that an antibody is typically at least about 50% w/w pure of interfering proteins and other contaminants arising from its production or purification but does not exclude the possibility that the antibody is combined with an excess of pharmaceutical acceptable carrier(s) or other vehicle intended to facilitate its use. Sometimes antibodies are at least about 60%, about 70%, about 80%, about 90%, about 95% or about 99% w/w pure of interfering proteins and contaminants from production or purification. Antibodies, including isolated antibodies, can be conjugated to cytotoxic agents and provided as antibody drug conjugates.

A “leader peptide” or “leader sequence” or “leader signal sequence” or “signal sequence” or “signal peptide”, (used interchangeably herein) refers any nucleic acid sequence, or amino acid sequence encoded thereby, that may be present on the 5′ end of a nucleic acid molecule or at the N-terminus of a polypeptide that when present may mediate the transport of the polypeptide to an organelle of destination, including, but not limited to, the secretion of the polypeptide from a cell. Such leader sequences include, but are not limited to, SEQ ID NO: 24 and SEQ ID NO: 25. The invention encompasses these and any other leader signals (nucleic acid and amino acid sequences) known in the art, or to be identified, which can result in the transport of a polypeptide to the desired organelle, e.g., the endoplasmic reticulum, e.g., secreted from the cell. Generally, the signal peptide is removed from and is not present in the mature polypeptide.

The term “binding affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art. Low-affinity antibodies generally bind antigen slowly and tend to dissociate readily, whereas high-affinity antibodies generally bind antigen faster and tend to remain bound longer. In particular, the term “binding affinity” is intended to refer to the dissociation rate of a particular antigen-antibody interaction. The K_{D} is the ratio of the rate of dissociation, also called the “off-rate (k_{off})” or “k_{d}” to the association rate, or “on-rate (kₒₙ)” or “kₐ”. Thus, K_{D} equals k_{off}/Kₒₙ (or k_{d}/kₐ) and is expressed as a molar concentration (M). It follows that the smaller the K_{D}, the stronger the affinity of binding. Therefore, a K_{D} of 1 μM indicates weaker binding affinity compared to a K_{D} of 1 nM. K_{D} values for antibodies can be determined using methods well established in the art. One exemplary method for determining the K_{D} of an antibody is by using surface plasmon resonance (SPR), typically using a biosensor system such as BIACORE system. BIACORE kinetic analysis comprises analyzing the binding and dissociation of an antigen from chips with immobilized molecules (e.g., molecules comprising epitope binding domains), on their surface. Another method for determining the K_{D} of an antibody is by using Bio-Layer Interferometry, typically using OCTET® technology (Octet QK^{e} system, ForteBio). Alternatively, or in addition, a KinExA (Kinetic Exclusion Assay) assay, available from Sapidyne Instruments (Boise, ID) can also be used.

Specific binding of an antibody to its target antigen typically refers an affinity of at least about 10⁶, about 10⁷, about 10⁸, about 10⁹, or about 10¹⁰ M⁻¹. The antibodies described herein typically bind the target antigen (e.g., CEACAM5) with an equilibrium binding constant of about ≤1 μM, e.g., about ≤100 nM, about ≤10 nM, or about ≤1 nM. Specific binding is detectably higher in magnitude and distinguishable from non-specific binding occurring to at least one non-specific target. Specific binding can be the result of formation of bonds between particular functional groups or particular spatial fit (e.g., lock and key type), whereas nonspecific binding is typically the result of van der Waals forces.

An “epitope” refers to the area or region of an antigen to which an antibody binds, e.g., an area or region comprising residues that interact with the antibody, as determined by any method well known in the art. There are many methods known in the art for mapping and characterizing the location of epitopes on proteins, including solving the crystal structure of an antibody-antigen complex, competition assays, gene fragment expression assays, epitope mapping, and synthetic peptide-based assays, as described, for example, in Chapter 11 of Harlow and Lane, Using Antibodies, a Laboratory Manual, Cold Spring Harbor Laboratory Press, Cold Spring Harbor, New York, 1999. In addition, or alternatively, during the discovery process, the generation and characterization of antibodies may elucidate information about desirable epitopes. From this information, it is then possible to competitively screen antibodies for binding to the same epitope.

In addition, the epitope to which an antibody binds can be determined in a systematic screening by using overlapping peptides derived from the antigen and determining binding by the antibody. According to the gene fragment expression assays, the open reading frame encoding the antigen can be fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis.

Certain epitopes can also be identified by using large libraries of random peptide sequences displayed on the surface of phage particles (phage libraries) or yeast (yeast display). Alternatively, a defined library of overlapping peptide fragments can be tested for binding to the test antibody in simple binding assays. In an additional example, mutagenesis of an antigen, domain swapping experiments and alanine scanning mutagenesis can be performed to identify residues required, sufficient, or necessary for epitope binding.

At its most detailed level, the epitope for the interaction between the antigen and the antibody can be defined by the spatial coordinates defining the atomic contacts present in the antigen-antibody interaction, as well as information about their relative contributions to the binding thermodynamics. At a less detailed level, the epitope can be characterized by the spatial coordinates defining the atomic contacts between the antigen and antibody. At a further less detailed level the epitope can be characterized by the amino acid residues that it comprises as defined by a specific criterion, e.g., by distance between atoms (e.g., heavy, i.e., non-hydrogen atoms) in the antibody and the antigen. At a further less detailed level the epitope can be characterized through function, e.g., by competition binding with other antibodies. The epitope can also be defined more generically as comprising amino acid residues for which substitution by another amino acid will alter the characteristics of the interaction between the antibody and antigen (e.g., using alanine scanning).

From the fact that descriptions and definitions of epitopes, dependent on the epitope mapping method used, are obtained at different levels of detail, it follows that comparison of epitopes for different antibodies on the same antigen can similarly be conducted at different levels of detail.

Epitopes described at the amino acid level, e.g., determined from an X-ray crystallography, Nuclear Magnetic Resonance (NMR) spectroscopy, hydrogen/deuterium exchange Mass Spectrometry (H/D-MS), are said to be identical if they contain the same set of amino acid residues. Epitopes are said to overlap if at least one amino acid is shared by the epitopes. Epitopes are said to be separate (unique) if no amino acid residue is shared by the epitopes.

Yet another method which can be used to characterize an antibody is to use competition assays with other antibodies known to bind to the same antigen, to determine if an antibody of interest binds to the same epitope as other antibodies. Competition assays are well known to those of skill in the art. Epitopes characterized by competition binding are said to be overlapping if the binding of the corresponding antibodies are mutually exclusive, i.e., binding of one antibody excludes simultaneous or consecutive binding of the other antibody. The epitopes are said to be separate (unique) if the antigen is able to accommodate binding of both corresponding antibodies simultaneously.

Epitopes can be linear or conformational. In a linear epitope, all of the points of interaction between the protein and the interacting molecule (such as an antibody) occur linearly along the primary amino acid sequence of the protein. A “nonlinear epitope” or “conformational epitope” comprises noncontiguous polypeptides (or amino acids) within the antigenic protein to which an antibody specific to the epitope binds.

Accordingly, antibodies that recognize the same or overlapping epitopes can be identified in a simple immunoassay showing the ability of one antibody to compete with the binding of another antibody to a target antigen. The epitope of an antibody can also be defined by X-ray crystallography of the antibody bound to its antigen to identify contact residues.

Alternatively, two antibodies have the same epitope if all amino acid mutations in the antigen that reduce or eliminate binding of one antibody reduce or eliminate binding of the other (provided that such mutations do not produce a global alteration in antigen structure). Two antibodies have overlapping epitopes if some amino acid mutations that reduce or eliminate binding of one antibody reduce or eliminate binding of the other antibody.

Competition between antibodies can be determined by an assay in which a test antibody inhibits specific binding of a reference antibody to a common antigen (see, e.g., Junghans et al., Cancer Res. 50:1495, 1990). A test antibody competes with a reference antibody if an excess of a test antibody inhibits binding of the reference antibody.

Antibodies identified by competition assay (competing antibodies) include antibodies that bind to the same epitope as the reference antibody and antibodies that bind to an adjacent epitope sufficiently proximal to the epitope bound by the reference antibody for steric hindrance to occur. Antibodies identified by a competition assay also include those that indirectly compete with a reference antibody by causing a conformational change in the target protein thereby preventing binding of the reference antibody to a different epitope than that bound by the test antibody.

An antibody effector function refers to a function contributed by an Fc region of an Ig. Such functions can be, for example, antibody-dependent cellular cytotoxicity (ADCC), antibody-dependent cellular phagocytosis (ADCP), or complement-dependent cytotoxicity (CDC). Such function can be affected by, for example, binding of an Fc region to an Fc receptor on an immune cell with phagocytic or lytic activity or by binding of an Fc region to components of the complement system. Typically, the effect(s) mediated by the Fc-binding cells or complement components result in inhibition and/or depletion of the CEACAM5-targeted cell. Fc regions of antibodies can recruit Fc receptor (FcR)-expressing cells and juxtapose them with antibody-coated target cells. Cells expressing surface FcR for IgGs including FcγRIII (CD16), FcγRII (CD32) and FcγRIII (CD64) can act as effector cells for the destruction of IgG-coated cells. Such effector cells include monocytes, macrophages, natural killer (NK) cells, neutrophils and eosinophils. Engagement of FcγR by IgG activates ADCC or ADCP. ADCC is mediated by CD16+ effector cells through the secretion of membrane pore-forming proteins and proteases, while phagocytosis is mediated by CD32+ and CD64+ effector cells (see Fundamental Immunology, 4^{th} ed., Paul ed., Lippincott-Raven, N.Y., 1997, Chapters 3, 17 and 30; Uchida et al., J. Exp. Med. 199: 1659-69, 2004; Akewanlop et al., Cancer Res. 61: 4061-65, 2001; Watanabe et al., Breast Cancer Res. Treat. 53:199-207, 1999).

In addition to ADCC and ADCP, Fc regions of cell-bound antibodies can also activate the complement classical pathway to elicit CDC. C1q of the complement system binds to the Fc regions of antibodies when they are complexed with antigens. Binding of C1q to cell-bound antibodies can initiate a cascade of events involving the proteolytic activation of C4 and C2 to generate the C3 convertase. Cleavage of C3 to C3b by C3 convertase enables the activation of terminal complement components including C5b, C6, C7, C8 and C9. Collectively, these proteins form membrane-attack complex pores on the antibody-coated cells. These pores disrupt the cell membrane integrity, killing the target cell (see Immunobiology, 6th ed., Janeway et al, Garland Science, N. Y., 2005, Chapter 2).

The term “antibody-dependent cell-mediated cytotoxicity” or “ADCC” refers to a form of cytotoxicity in which secreted Ig bound onto Fc receptors (FcRs) present on certain cytotoxic cells (e.g., NK cells, neutrophils, and macrophages) enable these cytotoxic effector cells to bind specifically to an antigen-bearing target cell and subsequently kill the target cell with cytotoxins. The primary cells for mediating ADCC, NK cells, express FcgRIII only, whereas monocytes express FcgRI, FcgRII, and FcgRIII. To assess ADCC activity of a molecule of interest, an in vitro ADCC assay, such as that described in U.S. Pat. Nos. 5,500,362, 5,821,337 or 6,737,056, may be performed. Useful effector cells for such assays include PBMC and NK cells. Alternatively, or additionally, ADCC activity of the molecule of interest may be assessed in vivo, e.g., in an animal model such as that disclosed in Clynes et al., Proc. Natl. Acad. Sci. (USA) 1998; 95:652-656. Additional antibodies with altered Fc region amino acid sequences and increased or decreased ADCC activity are described, e.g., in U.S. Pat. Nos. 7,923,538, and 7,994,290.

The term “antibody-dependent cellular phagocytosis” or “ADCP” refers to the process by which antibody-coated cells are internalized, either in whole or in part, by phagocytic immune cells (e.g., by macrophages, neutrophils and/or dendritic cells) that bind to an Fc region of Ig.

The term “complement dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, e.g., as described in Gazzano-Santoro et al., J. Immunol. Methods 1996; 202:163, may be performed. Antibodies with altered Fc region amino acid sequences and increased or decreased C1q binding capability are described, e.g., in U.S. Pat. Nos. 6,194,551, 7,923,538, 7,994,290 and WO 1999/51642.

Typically, antigen-antibody complexes such as those on antibody-coated target cells bind and activate complement component C1q, which in turn activates the complement cascade leading to target cell death. Activation of complement may also result in deposition of complement components on the target cell surface that facilitate ADCC by binding complement receptors (e.g., CR3) on leukocytes.

The term “compete,” as used herein with regard to an antibody, means that a first antibody binds to an epitope in a manner sufficiently similar to the binding of a second antibody such that the result of binding of the second antibody with its cognate epitope is detectably decreased in the presence of the first antibody compared to the binding of the second antibody in the absence of the first antibody. The alternative, where the binding of the first antibody to its epitope is also detectably decreased in the presence of the second antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing or cross-competing antibodies are encompassed and can be useful for the methods disclosed herein.

Standard competition assays may be used to determine whether two antibodies compete with each other. One suitable assay for antibody competition involves the use of the Biacore technology, which can measure the extent of interactions using surface plasmon resonance (SPR) technology, typically using a biosensor system (such as a BIACORE system). For example, SPR can be used in an in vitro competitive binding inhibition assay to determine the ability of one antibody to inhibit the binding of a second antibody. Another assay for measuring antibody competition uses an ELISA-based approach.

Furthermore, a high throughput process for “binning” antibodies based upon their competition is described in International Patent Application No. WO2003/48731. Competition is present if one antibody (or fragment) reduces the binding of another antibody (or fragment) to an epitope or target of interest. For example, a sequential binding competition assay may be used, with different antibodies being added sequentially. The first antibody may be added to reach binding that is close to saturation. Then, the second antibody is added. If the binding of second antibody to the epitope is not detected, or is significantly reduced (e.g., at least about 10%, at least about 20%, at least about 30%, at least about 40%, at least about 50%, at least about 60%, at least about 70%, at least about 80%, or at least about 90% reduction) as compared to a parallel assay in the absence of the first antibody (which value can be set as 100%), the two antibodies are considered as competing with each other.

A “cytotoxic effect” refers to the depletion, elimination and/or killing of a target cell. A “cytotoxic agent” refers to a compound that has a cytotoxic effect on a cell, thereby mediating depletion, elimination and/or killing of a target cell. In certain embodiments, a cytotoxic agent is conjugated to an antibody or administered in combination with an antibody. Suitable cytotoxic agents are described further herein.

A “cytostatic effect” refers to the inhibition of cell proliferation. A “cytostatic agent” refers to a compound that has a cytostatic effect on a cell, thereby mediating inhibition of growth and/or expansion of a specific cell type and/or subset of cells. Suitable cytostatic agents are described further herein.

The terms “subject, “individual” or “patient,” (used interchangeably herein), refer to any animal, including mammals. Mammals according to the invention include canine, feline, bovine, caprine, equine, ovine, porcine, rodents, lagomorphs, primates, humans and the like, and encompass mammals in utero. A “subject, “individual” or “patient,” can refer to organisms to be treated by the methods of the present invention. In an embodiment, humans are suitable subjects. Human subjects may be of any gender and at any stage of development. In some embodiments, a subject is a patient with cancer.

As used herein, the terms “treat,” “treatment” and “treating” include any effect, e.g., lessening, reducing, modulating, ameliorating or eliminating, that results in the improvement of the condition, disease, disorder, and the like, or ameliorating a symptom thereof, such as for example, reduced number of cancer cells, reduced tumor size, reduced rate of cancer cell infiltration into peripheral organs, or reduced rate of tumor metastasis or tumor growth. For example, the terms “treating”, “treat” or “treatment” can refer to an approach for obtaining clinical benefit or desired clinical results, e.g. such as to partially or completely relieve, alleviate, ameliorate, inhibit, delay the onset of, reduce the severity of, reduce the frequency of, reduce the incidence of, slow the progression of, or any combination thereof, of one or more symptoms of the patient's disease, disorder or condition or any tissue damage associated with the disease. Treatment may also include improved survival rate, decreased duration of the disease, reduction in the number, extent or duration of symptoms of the disease, disorder or condition. Treatment may be prophylactic (to delay the onset of the disease, or to prevent the manifestation of at least one clinical symptom thereof) or therapeutic suppression of or alleviation of a symptom after manifestation of the disease. In some embodiments, the disease, disorder or condition is cancer.

The terms “prevent” or “prevention” refer to preventing a disease, condition or disorder in an individual that may be predisposed to the disease, condition or disorder but does not yet experience or display the pathology or symptomatology of the disease. In some embodiments, prevention is assessed on a population basis such that an agent is considered to “prevent” a particular disease, disorder or condition if a statistically significant decrease in the development, frequency or intensity of the disease, disorder or condition is observed in a population susceptible to the disease, disorder or condition. Prevention may be considered complete when onset of disease, disorder or condition has been delayed for a predefined period of time.

“Tumor” as it applies to a subject diagnosed with, or suspected of having, cancer (e.g., solid cancer or breast cancer), refers to a malignant or potentially malignant neoplasm or tissue mass of any size.

“Tumor burden” also referred to as “tumor load,” refers to the total amount of tumor material distributed throughout the body. Tumor burden refers to the total number of cancer cells or the total size of tumor(s) throughout the body, including lymph nodes and bone narrow. Tumor burden can be determined by a variety of methods known in the art, such as, e.g., by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g., by measuring the dimensions of tumor(s) upon removal from the subject, e.g., using calipers, or while in the body using imaging techniques, e.g., bone scan, ultrasound, CT or MRI scans.

The term “therapeutically effective amount” or “effective amount” refers to the amount of a compound or active ingredient (e.g., an anti-CEACAM5 antibody or antibody-drug conjugate) sufficient to effect beneficial or desired results, or elicits a biological or medicinal response in a tissue, system, animal, individual or human that is being sought by a researcher, veterinarian, medical doctor or other clinician, which may include one or more of the following: (1) preventing the disease; for example, preventing a disease, condition or disorder in an individual that may be predisposed to the disease, condition or disorder but does not yet experience or display the pathology or symptomatology of the disease; (2) inhibiting the disease; for example, inhibiting a disease, condition or disorder in an individual that is experiencing or displaying the pathology or symptomatology of the disease, condition or disorder (i.e., arresting or slowing further development of the pathology or symptomatology); and (3) ameliorating the disease; for example, ameliorating a disease, condition or disorder in an individual that is experiencing or displaying the pathology or symptomatology of the disease, condition or disorder (i.e., reversing the pathology or symptomatology). In some embodiments, an effective amount of an antibody or antibody-drug conjugate (e.g., a CEACAM5-ADC) is administered in one or more administrations, applications or dosages and is not intended to be limited to a particular formulation or administration route.

The term “pharmaceutically acceptable” means approved or approvable by a regulatory agency of the Federal or a state government or listed in the U.S. Pharmacopeia or other generally recognized pharmacopeia for use in animals, and more particularly in humans. The term “pharmaceutically compatible ingredient” refers to a pharmaceutically acceptable diluent, adjuvant, excipient, or vehicle with which an anti-CEACAM5 antibody (e.g., a CEACAM5-ADC) is formulated.

The term “excipient” refers to any material which, which combined with an active ingredient of interest (e.g., antibody), allow the active ingredient to retain biological activity. The choice of excipient will to a large extent depend on factors such as the mode of administration, the effect of the excipient on solubility and stability, and the nature of the dosage form. As used herein, “excipient” “includes any and all solvents, dispersion media, coatings, antibacterial and antifungal agents, isotonic and absorption delaying agents, carriers, diluents and the like that are physiologically compatible. Examples of an excipient include one or more of water, saline, phosphate buffered saline, dextrose, glycerol, ethanol and the like, as well as combinations thereof, and may include isotonic agents, for example, sugars, sodium chloride, or polyalcohols such as mannitol, or sorbitol in the composition.

The phrase “pharmaceutically acceptable salt,” refers to pharmaceutically acceptable organic or inorganic salts. Exemplary salts include sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p toluenesulfonate, and pamoate (i.e., 1,1′-methylene bis-(2 hydroxy-3-naphthoate) salts. A pharmaceutically acceptable salt may further comprise an additional molecule such as, e.g., an acetate ion, a succinate ion or other counterion. A counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

Unless otherwise apparent from the context, when a value is expressed as “about” X or “approximately” X, the stated value of X will be understood to be accurate to ±10%.

Solvates in the context of the invention are those forms of the compounds of the invention that form a complex in the solid or liquid state through coordination with solvent molecules. Hydrates are one specific form of solvates, in which the coordination takes place with water. In certain exemplary embodiments, solvates in the context of the present invention are hydrates.

### Antibodies to CEACAM5

The disclosure provides antibodies that bind to CEACAM5 (Carcinoembryonic Antigen Cell Adhesion Molecule 5) and compositions, methods and uses thereof, including use of the antibodies of the disclosure to treat cancer.

As used herein, the term CEACAM5 includes variants, isoforms, homologs, orthologs and paralogs of CEACAM5, wherein CEACAM5 refers to naturally occurring human CEACAM5 unless contextually dictated otherwise. Therefore, a “CEACAM5 antibody” “anti-CEACAM5 antibody” or other similar designation means any antibody (as defined herein) that binds or reacts with CEACAM5, an isoform, fragment or derivative thereof.

The full length, mature form of human CEACAM5 (hCEACAM5), as represented by UniProt accession number P06731 is herein provided as SEQ ID NO: 1, with the extracellular domain (ECD) consisting of the amino acids 35-685 of SEQ ID NO: 1 (SEQ ID NO: 3). The full length, mature form of cynomolgus monkey CEACAM5 (cyno CECAM5 or cCEACAM5), as represented by UniProtKB/Swiss-Prot accession number 005589491 is herein provided as SEQ ID NO: 2, with the ECD consisting of amino acids 35-688 of SEQ ID NO: 2 (SEQ ID NO: 4).

The ECD of CEACAM5 comprises an Ig variable region (IgV)-like domain, termed N, followed by six Ig constant region (IgC)-type 2-like domains, termed A1, B1, A2, B2, A3, and B3, wherein amino acids 35-142, 143-237, 238-320, 321-415, 415-498, 499-593 and 594-685 of SEQ ID NO: 1, corresponds to each domain of hCEACAM5, respectively.

In some embodiments, an antibody disclosed herein cross-reacts with CEACAM5 from species other than human, such as CEACAM5 of cynomolgus monkey (cynomolgus or “cyno”), as well as different forms of CEACAM5. In some embodiments, an antibody may be specific for human CEACAM5 and may not exhibit species cross-reactivity (e.g., does not bind cynomolgus CEACAM5) or other types of cross-reactivity (e.g., does not bind human CEACAM1, CEACAM6, CEACAM7, or CEACAM8, in which the full length sequences and ECDs of each are available in GenBank database, under accession number NP_001703.2 (SEQ ID NO: 26, with ECD consisting of amino acids 35-428 of SEQ ID NO: 26), under accession number NP_002474.4 (SEQ ID NO:27, with ECD consisting of amino acids 35-327 of SEQ ID NO: 27), under accession number NP_008821.2 (SEQ ID NO:28, with ECD consisting of amino acids 35-248 of SEQ ID NO: 28), and under accession number NP_001807.2 (SEQ ID NO:29, with ECD consisting of amino acids 35-332 of SEQ ID NO: 29), respectively). For example, an antibody may exhibit binding specific for human CEACAM5 and the antibody does not bind cyno CEACAM5. The antibody may also further not bind to other CEACAM members, such as human CEACAM1, CEACAM6, CEACAM7, or CEACAM8.

In some embodiments, an antibody may bind the ECD of CEACAM5. The antibody may be specific to the ECD of human CEACAM5 and not exhibit species cross-reactivity (e.g., does not bind the ECD of cyno CEACAM5) or other types of cross-reactivity (e.g., does not bind the ECD of human CEACAM1, CEACAM6, CEACAM7, or CEACAM8). For example, an antibody may exhibit binding specific for the ECD of human CEACAM5 and the antibody does not bind the ECD of cyno CEACAM5. The antibody may also not bind the ECD of other CEACAM members, such as the ECD of human CEACAM1, CEACAM6, CEACAM7, or CEACAM8.

In some embodiments, an antibody may bind one or more domains, e.g., N, A1, B1, A2, B2, A3, and/or B3 domain(s) of CEACAM5. In one embodiment, a CEACAM5 antibody does not bind to the A2 and B2 domains. In another embodiment, a CEACAM5 antibody does not bind to the A3 and B3 domains. In a particular embodiment, a CEACAM5 antibody does not bind to a fragment comprising the A2 and B2 domains, such as a fragment comprising the amino acid sequence of SEQ ID NO: 6. In yet another embodiment, a CEACAM5 antibody does not bind to a fragment comprising the A3 and B3 domains, such as a fragment comprising the amino acid sequence of SEQ ID NO: 7. In one embodiment, a CEACAM5 antibody does not bind to a fragment comprising the A2 and B2 domains, such as a fragment comprising the amino acid sequence of SEQ ID NO: 6, and also does not bind to a fragment comprising the A3 and B3 domains, such as a fragment comprising the amino acid sequence of SEQ ID NO: 7. In some embodiments, a CEACAM5 antibody binds to the N, A1, and/or B1 domains, for example, a fragment comprising the amino acid sequence of SEQ ID NO: 5.

In some embodiments, an anti-CEACAM5 antibody of the disclosure encompasses an antibody that one or both of i) competes for binding to human CEACAM5 with or ii) binds the same epitope as, an antibody having the amino acid sequence of a heavy chain variable region set forth as SEQ ID NO:14 and the amino acid sequence of a light chain variable region set forth as SEQ ID NO: 15.

Anti-CEACAM5 antibodies of the present disclosure can encompass monoclonal antibodies, polyclonal antibodies, antibody fragments (e.g., Fab, Fab′, F(ab′)₂, Fv, Fc, etc.), chimeric antibodies, bispecific antibodies, heteroconjugate antibodies, single chain (ScFv), mutants thereof, fusion proteins comprising an antibody fragment (e.g., a domain antibody), humanized antibodies, and any other modified configuration of the immunoglobulin molecule that comprises an antigen binding site of the required specificity, including glycosylation variants of antibodies, amino acid sequence variants of antibodies, and covalently modified antibodies. The antibodies may be murine, rat, human, or any other origin (including chimeric or humanized antibodies). In some embodiments, an anti-CEACAM5 antibody is a monoclonal antibody. In some embodiments, an anti-CEACAM5 antibody is a human or humanized antibody. In a particular embodiment, the anti-CEACAM5 antibody is a human antibody. In some embodiments, an anti-CEACAM5 antibody is a chimeric antibody.

In some embodiments, the invention provides an antibody having a heavy chain variable region (VL) sequence of SEQ ID NO: 14 and a light chain variable region (VH) sequence of SEQ ID NO: 15. The invention also provides CDR portions of antibodies to CEACAM5. Determination of CDR regions is well within the skill of the art. It is understood that in some embodiments, CDRs can be a combination of the Kabat and Chothia CDR (also termed “combined CDRs” or “extended CDRs”). In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. In general, “conformational CDRs” include the residue positions in the Kabat CDRs and Vernier zones which are constrained in order to maintain proper loop structure for the antibody to bind a specific antigen. Determination of conformational CDRs is well within the skill of the art. In some embodiments, the CDRs are the Kabat CDRs. In other embodiments, the CDRs are the Chothia CDRs. In other embodiments, the CDRs are the extended, AbM, conformational, or contact CDRs. In other words, in embodiments with more than one CDR, the CDRs may be any of Kabat, Chothia, extended, AbM, conformational, contact CDRs or combinations thereof.

In some embodiments, the antibody comprises three CDRs of a heavy chain variable region comprising the amino acid sequence of SEQ ID NO: 14. In some embodiments, the antibody comprises three CDRs of the light chain variable region comprising the amino acid sequence of SEQ ID NO: 15. In some embodiments, the antibody comprises one or both of i) the full-length heavy chain, with or without the C-terminal lysine, and ii) the full-length light chain, of anti-CEACAM5 antibody SG-105.2E4. The amino acid sequences of the full-length heavy chain and light chain for SG-105.2E4 is SEQ ID NO: 18 (SEQ ID NO: 31 without the C-terminal lysine) and SEQ ID NO: 20, respectively. In some embodiments, the antibody comprises one or both of i) the full-length heavy chain, with or without the C-terminal lysine, and ii) the full-length light chain, of anti-CEACAM5 antibody SG-105.2E4 EC4. The amino acid sequences of the full-length heavy chain and light chain for SG-105.2E4 EC4 is SEQ ID NO: 22 (SEQ ID NO: 33 without the C-terminal lysine) and SEQ ID NO: 23, respectively.

In certain embodiments, an antibody described herein comprises an Fc domain. The Fc domain can be derived from IgA (e.g., IgA1 or IgA2), IgG, IgE, or IgG (e.g., IgG1, IgG2, IgG3, or IgG4). In some embodiments, an anti-CEACAM5 antibody is an IgG1 antibody.

In some embodiments, the variable regions comprise one or more modifications. For example, the invention includes antibodies comprising functionally equivalent variable regions and CDRs which do not significantly affect their properties as well as variants which have enhanced or decreased activity or affinity. For example, the amino acid sequence may be mutated to obtain an antibody with the desired binding affinity to CEACAM5. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or which mature (enhance) the affinity of the polypeptide for its ligand, or use of chemical analogs.

A modification or mutation may also be made in a framework region or constant region to increase the half-life of an antibody provided herein. See, e.g., PCT Publication No. WO 00/09560. A mutation in a framework region or constant region can also be made to alter the immunogenicity of the antibody, to provide a site for covalent or non-covalent binding to another molecule, or to alter such properties as complement fixation, FcR binding and antibody-dependent cell-mediated cytotoxicity. In some embodiments, no more than one to five conservative amino acid substitutions are made within the framework region or constant region. In other embodiments, no more than one to three conservative amino acid substitutions are made within the framework region or constant region. According to the invention, a single antibody may have mutations in any one or more of the CDRs or framework regions of the variable domain or in the constant region.

In some embodiments, the antibody comprises a modified constant region that has increased or decreased binding affinity to a human Fc gamma receptor, is immunologically inert or partially inert, e.g., does not trigger complement mediated lysis, does not stimulate antibody-dependent cell mediated cytotoxicity (ADCC), or does not activate microglia; or has reduced activities (compared to the unmodified antibody) in any one or more of the following: triggering complement mediated lysis, stimulating ADCC, or activating microglia. Different modifications of the constant region may be used to achieve optimal level or combination of effector functions. See, for example, Morgan et al., Immunology 86:319-324, 1995; Lund et al., J. Immunology 157:4963-9 157:4963-4969, 1996; Idusogie et al., J. Immunology 164:4178-4184, 2000; Tao et al., J. Immunology 143:2595-2601, 1989; and Jefferis et al., Immunological Reviews 163:59-76, 1998. In some embodiments, the constant region is modified as described in Eur. J. Immunol., 1999, 29:2613-2624; PCT Publication No. WO99/058572.

In some embodiments, the antibody with modified constant region is a cysteine engineered antibody, such as described in PCT application number PCT/US2023/083664. In some embodiments, the cysteine amino acid substitution is at position 114 of each of the light chains and position 400 of each of the heavy chains, wherein the amino acid position numbering in the light chains is according to Kabat numbering and the amino acid position numbering in the heavy chains are according to EU numbering. The antibody can comprise a heavy chain and a light chain, wherein the cysteine amino acid substitution is at position 114 (e.g., S114C) of the light chain, according to the Kabat numbering convention for antibodies, and the cysteine amino acid substitution is at position 400 (e.g., S400C) of the heavy chain, according to the EU numbering convention for antibodies. In some embodiments, the antibody can comprise two heavy chains and two light chains, wherein the cysteine amino acid substitutions is at position 114 (e.g., S114C) of each of the light chains, according to the Kabat numbering convention for antibodies, and the cysteine amino acid substitutions are at position 400 (e.g., S400C) of each of the heavy chains, according to the EU numbering convention for antibodies.

In one embodiment, the cysteine engineered heavy chain constant region comprises the amino acid sequence of SEQ ID NO: 20. In one embodiment, the cysteine engineered light chain constant region comprises the amino acid sequence of SEQ ID NO: 21. Accordingly in some embodiments, the antibody with modified constant region comprises a heavy chain constant chain comprises the amino acid sequence of SEQ ID NO: 20 and a light constant chain comprises the amino acid sequence of SEQ ID NO: 21. In some embodiments, the antibody can comprise two heavy chains, each comprising the amino acid sequence of SEQ ID NO: 20, and two light chains, each comprising the amino acid sequence of SEQ ID NO: 21.

Modifications also include glycosylated and nonglycosylated polypeptides, as well as polypeptides with other post-translational modifications, such as, for example, glycosylation with different sugars, acetylation, and phosphorylation. Antibodies are glycosylated at conserved positions in their constant regions (Jefferis and Lund, 1997, Chem. Immunol. 65:111-128; Wright and Morrison, 1997, TibTECH 15:26-32). The oligosaccharide side chains of the immunoglobulins affect the protein's function (Boyd et al., 1996, Mol. Immunol. 32:1311-1318; Wittwe and Howard, 1990, Biochem. 29:4175-4180) and the intramolecular interaction between portions of the glycoprotein, which can affect the conformation and presented three-dimensional surface of the glycoprotein (Jefferis and Lund, supra; Wyss and Wagner, 1996, Current Opin. Biotech. 7:409-416). Oligosaccharides may also serve to target a given glycoprotein to certain molecules based upon specific recognition structures. Glycosylation of antibodies has also been reported to affect antibody-dependent cellular cytotoxicity (ADCC). In particular, antibodies produced by CHO cells with tetracycline-regulated expression of β (1,4)-N-acetylglucosaminyltransferase III (GnTIII), a glycosyltransferase catalyzing formation of bisecting GlcNAc, was reported to have improved ADCC activity (Umana et al., 1999, Nature Biotech. 17:176-180).

In some embodiments, the disclosure provides anti-CEACAM5 antibodies containing variations of the variable regions of the heavy or light chain comprising the amino acid sequence of SEQ ID NO: 14 and SEQ ID NO: 15, respectively, wherein such variant polypeptides share at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% amino acid sequence identity to SEQ ID NO: 14 or SEQ ID NO: 15. These amounts are not meant to be limiting and increments between the recited percentages are specifically envisioned as part of the disclosure.

In some embodiments provided herein is an anti-CEACAM5 antibody that comprises a heavy chain and a light chain, wherein the antibody heavy chain has an amino acid sequence of SEQ ID NO: 18 and the antibody light chain has an amino acid sequence of SEQ ID NO: 19. In some embodiments provided herein is an anti-CEACAM5 antibody that comprises a heavy chain and a light chain, wherein the antibody heavy chain has an amino acid sequence of SEQ ID NO: 22 and the antibody light chain has an amino acid sequence of SEQ ID NO: 23.

In some embodiments provided herein is an anti-CEACAM5 antibody that comprises a heavy chain and a light chain, wherein the antibody heavy chain has an amino acid sequence of SEQ ID NO: 31 and the antibody light chain has an amino acid sequence of SEQ ID NO: 19. In some embodiments provided herein is an anti-CEACAM5 antibody that comprises a heavy chain and a light chain, wherein the antibody heavy chain has an amino acid sequence of SEQ ID NO: 33 and the antibody light chain has an amino acid sequence of SEQ ID NO: 23.

The present invention provides isolated, recombinant and/or synthetic human, primate, rodent, mammalian, chimeric, humanized and/or CDR-grafted antibodies and antigen-binding fragments and antibody-drug conjugates (e.g., a CEACAM5-ADC) thereof, as well as compositions and nucleic acid molecules comprising at least one polynucleotide encoding at least a portion of one antibody molecule.

The disclosure provides polynucleotides encoding any of the antibodies of the invention, including antibody portions and modified antibodies described herein. The invention also provides a method of making any of the antibodies and polynucleotides described herein. Polynucleotides can be made and the proteins expressed by procedures known in the art.

If desired, an anti-CEACAM5 antibody (monoclonal or polyclonal) of interest may be sequenced and the polynucleotide sequence may then be cloned into a vector for expression or propagation. The sequence encoding the antibody of interest may be maintained in vector in a host cell and the host cell can then be expanded and frozen for future use. Production of recombinant monoclonal antibodies in cell culture can be carried out through cloning of antibody genes from B cells by means known in the art. See, e.g., Tiller et al., 2008, J. Immunol. Methods 329, 112; U.S. Pat. No. 7,314,622.

In some embodiments, provided herein is a polynucleotide comprising a sequence encoding one or both of the heavy chain or the light chain variable regions of an anti-CEACAM5 antibody provided herein. The sequence encoding the antibody of interest may be maintained in a vector in a host cell and the host cell can then be expanded and frozen for future use. Vectors (including expression vectors) and host cells are further described herein.

In some embodiments, the disclosure provides polynucleotides encoding one or more anti-CEACAM5 antibody heavy chain polypeptides comprising an amino acid sequence of SEQ ID NO: 18 or 22. In one particular embodiment, the polynucleotide encodes a heavy chain polypeptide comprising the amino acid sequence of SEQ ID NO: 22. In some embodiments, the disclosure provides polynucleotides encoding one or more anti-CEACAM5 antibody light chain polypeptides comprising an amino acid sequence of SEQ ID NO: 19 or 23. In one particular embodiment, the polynucleotide encodes a heavy chain polypeptide comprising the amino acid sequence of SEQ ID NO: 23.

In some embodiments, the disclosure provides polynucleotides encoding one or more anti-CEACAM5 antibody heavy chain polypeptides comprising an amino acid sequence of SEQ ID NO: 31 or 33. In one particular embodiment, the polynucleotide encodes a heavy chain polypeptide comprising the amino acid sequence of SEQ ID NO: 33. In some embodiments, the disclosure provides polynucleotides encoding one or more anti-CEACAM5 antibody light chain polypeptides comprising an amino acid sequence of SEQ ID NO: 19 or 23. In one particular embodiment, the polynucleotide encodes a heavy chain polypeptide comprising the amino acid sequence of SEQ ID NO: 23.

In some embodiments, the disclosure provides polynucleotides encoding an anti-CEACAM5 antibody VH polypeptide comprising an amino acid sequence of SEQ ID NO:14. In some embodiments, the disclosure provides polynucleotides encoding one or more anti-CEACAM5 antibody VL polypeptide comprising an amino acid sequence of: SEQ ID NO: 15.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions or substitutions of nucleotides. The resulting mRNA and protein may, but need not, have an altered structure or function. Alleles may be identified using standard techniques (such as hybridization, amplification or database sequence comparison).

In one embodiment, the VH and VL domains or full-length HC or LC, are encoded by separate polynucleotides. Alternatively, both VH and VL, or HC and LC, are encoded by a single polynucleotide.

Polynucleotides complementary to any such sequences are also encompassed by the present disclosure. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present disclosure, and a polynucleotide may, but need not, be linked to other molecules or support materials.

The polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome.

Suitable cloning vectors may be constructed according to standard techniques, or may be selected from a large number of cloning vectors available in the art. While the cloning vector selected may vary according to the host cell intended to be used, useful cloning vectors will generally have one or more features such as i) the ability to self-replicate, ii) a single target for a particular restriction endonuclease, or iii) may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors are further provided. Expression vectors generally are replicable polynucleotide constructs that contain a polynucleotide according to the invention. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

The invention also provides host cells comprising any of the polynucleotides described herein. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis).

Additionally, any number of commercially and non-commercially available cell lines that express polypeptides or proteins may be utilized in accordance with the present invention. One skilled in the art will appreciate that different cell lines might have different nutrition requirements or might require different culture conditions for optimal growth and polypeptide or protein expression, and will be able to modify conditions as needed.

The present invention further includes, but is not limited to, methods of making and using such nucleic acids and antibodies including diagnostic and therapeutic compositions, methods and devices.

In some embodiments, the antibody mediates ADCC activity against CEACAM5-expressing cells. In some embodiments, the antibody or antigen-binding fragment thereof does not mediate CDC activity against CEACAM5-expressing cells.

In some aspects, provided herein are methods of treating a subject with cancer or at risk of having cancer, comprising administering to the subject an effective amount of an antibody that specifically binds to CEACAM5. In some embodiments, an antibody-drug conjugate (ADC) comprising an antibody that specifically binds to CEACAM5.

### II. Antibody-Drug Conjugates

The disclosure also provides antibody-drug conjugates (ADCs) comprising antibodies that bind to CEACAM5 and compositions, methods and uses thereof, including use of the ADCs of the disclosure to treat cancer. In some embodiments, the antibodies of the invention (e.g., anti-CEACAM5 antibodies disclosed here) is conjugated to a drug to form ADCs (CEACAM5-ADCs).

In some embodiments, the CEACAM5-ADC is an ADC composed of a fully human IgG1 anti-CEACAM5 monoclonal antibody (mAb) conjugated to the microtubule disrupting agent monomethyl auristatin E (MMAE) via a protease-cleavable peptide linker (Doronina et al., 2003. Nat Biotechnol 21, 778-784). This “vedotin” drug linker system has been clinically validated by multiple ADC programs, including brentuximab vedotin (Adcetris™), enfortumab vedotin (PADCEV™), and polatuzumab vedotin (POLIVY™) (Rosenberg et al., 2019, J Clin Oncol 37, 2592-2600; Senter and Sievers, 2012, Nat Biotechnol 30, 631-637; Tilly et al., 2019, Lancet Oncol 20, 998-1010).

In certain embodiments, an antibody or antigen-binding fragment thereof can be conjugated to a drug to form an antibody-drug conjugate (ADC) and may have a ratio of drug moieties per antibody of about 1 to about 8, or about 1 to about 4. In certain embodiments, an antibody or antigen-binding fragment thereof (e.g., anti-CEACAM5 antibody) can be conjugated to a drug to form an ADC and may have a ratio of drug moieties per antibody of about 2 to about 5. In some embodiments, the ratio of drug moieties per antibody is 1, 2, 3, 4, 5, 6, 7, 8, 9, or 10. In certain exemplary embodiments, an anti-CEACAM5 antibody or antigen-binding fragment thereof can be conjugated to a drug to form an ADC and have a ratio of drug moieties per antibody of about 4. In some embodiments, the average number of drug moieties per antibody in a population of antibody-drug conjugates is about 1 to about 8. In some embodiments, the average number of drug moieties per antibody in a population of antibody-drug conjugates is about 1 to about 4. In some embodiments, the average number of drug moieties per antibody in a population of antibody-drug conjugates is about 4. Methods of determining the ratio of drug moieties per antibody or antigen-binding fragment thereof of an ADC are readily known to those skilled in the art.

According to certain exemplary embodiments, a CEACAM5-ADC comprises monomethyl auristatin E (MMAE) (PubChem CID: 53297465):

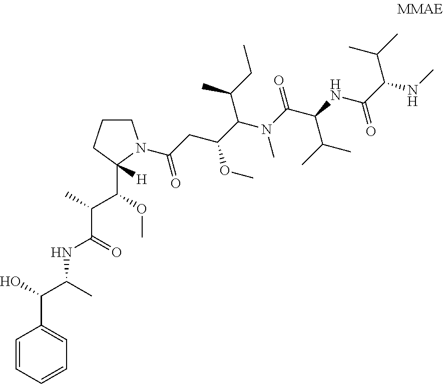 [SMILES: CC[C@H](C)C([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

According to certain exemplary embodiments, a CEACAM5-ADC comprises vcMMAE conjugated thereto. vcMMAE is a drug-linker conjugate for ADC with potent anti-tumor activity comprising the anti-mitotic agent, MMAE, linked via the lysosomally cleavable dipeptide valine-citrulline (vc):

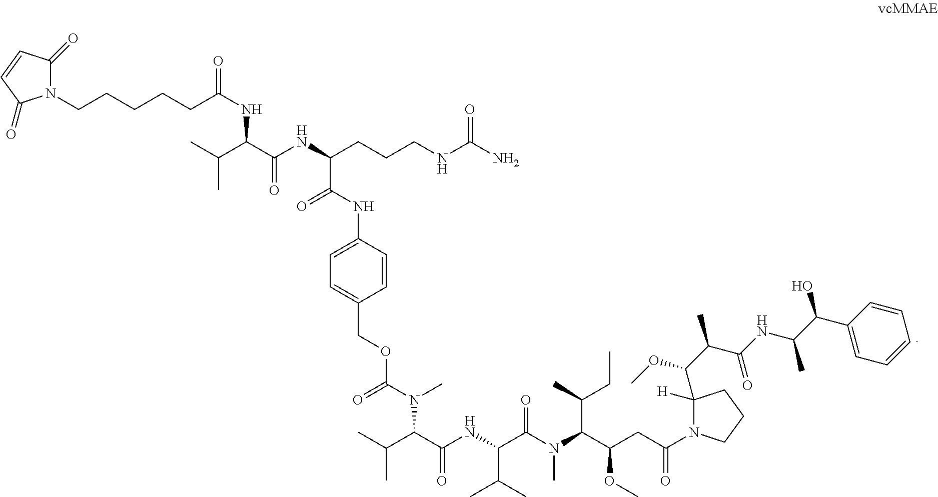 [SMILES: CC[C@H](C)[C@@H]([C@@H](CC(=O)N1CCCC1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

vcMMAE may also be referred to as MC-Val-Cit-PABC-MMAE, where MC refers to a maleimidocaproyl group, Val-Cit refers to the dipeptide valine-citrulline, PABC refers to a para-aminobenzylcarbamate group, and MMAE refers to the drug monomethyl auristatin E.

The structure of a vcMMAE-antibody conjugate (e.g., a CEACAM5-ADC) according to certain exemplary embodiments is set forth below. In this structure, the drug-linker portion shown within the parentheses may be referred to in some instances as vedotin. The drug-linker may be attached to the antibody via a sulfur atom of a cysteine residue of the antibody. In some embodiments, the ADC shown below is formed by reaction of the maleimide group of the vc-MMAE drug-linker precursor with a thiol of a cysteine residue of the antibody to form a succinamide bonded to the sulfur atom of the cysteine residue.

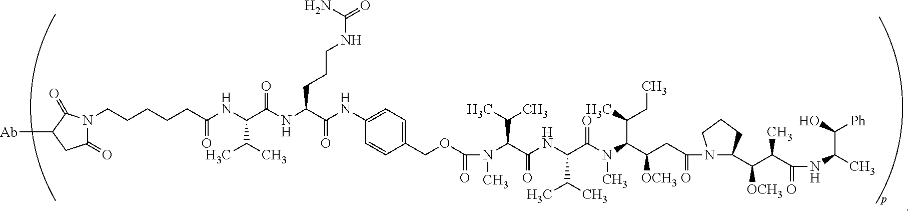

In some embodiments, the succinamide moiety of the ADC may undergo a ring-opening hydrolysis to form one of the ring-opened structures shown below.

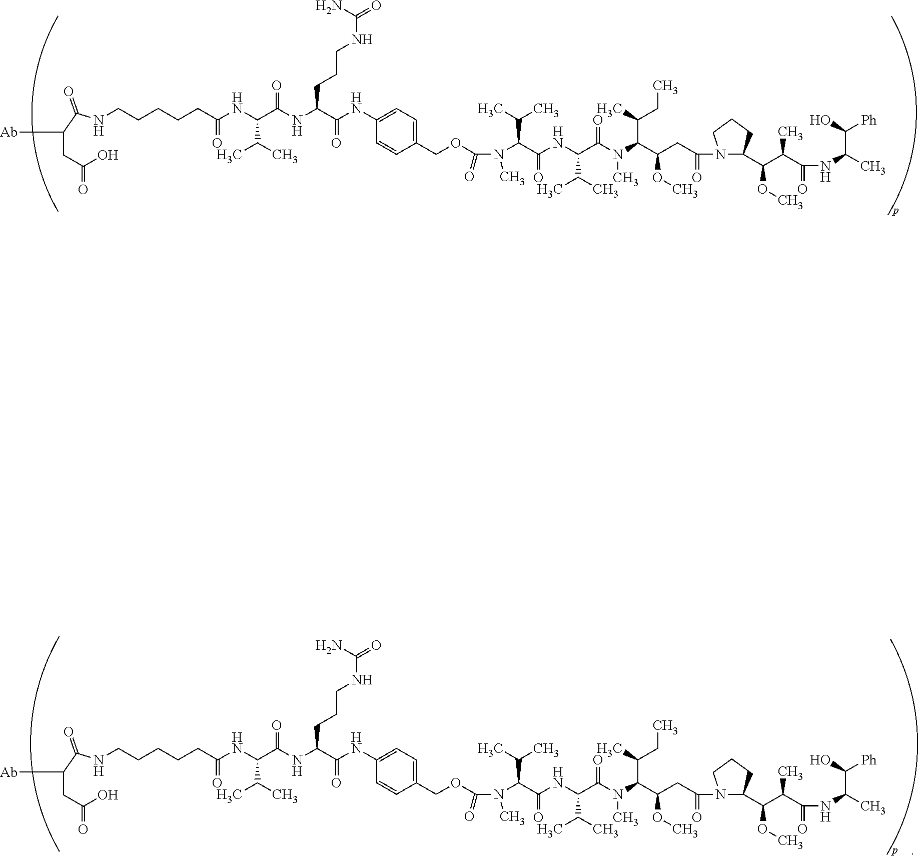

In some embodiments, p is about 1 to about 8. In some embodiments, p is about 1 to about 4. In some embodiments, p is about 4.

According to some embodiments, a vcMMAE-antibody conjugate (e.g., a CEACAM5-ADC) is provided as set forth above, wherein Ab may include an anti-CEACAM5 antibody (e.g., SG-105.2E4 or SG-105.2E4 EC antibody), and wherein p may be any integer from about 1 to about 8. In some embodiments, a vcMMAE-antibody conjugate (e.g., a CEACAM5-ADC) is provided as set forth above, wherein Ab may include an anti-CEACAM5 antibody (e.g., SG-105.2E4 or SG-105.2E4 EC antibody), and wherein p is from about 1 to about 4. In some embodiments, a vcMMAE-antibody conjugate (e.g., a CEACAM5-ADC) is provided as set forth above, wherein Ab may include an anti-CEACAM5 antibody (e.g., SG-105.2E4 or SG-105.2E4 EC antibody), and wherein p is 1, representing a vcMMAE to antibody or antigen-binding fragment thereof ratio of 1. In some embodiments, a vcMMAE-antibody conjugate (e.g., a CEACAM5-ADC) is provided as set forth above, wherein Ab may include an anti-CEACAM5 antibody (e.g., SG-105.2E4 or SG-105.2E4 EC antibody), and wherein p is 2, 3, 4, 5, 6, 7, 8, 9, or 10, representing a vcMMAE to antibody or antigen-binding fragment thereof ratio (also known as a “Drug-to-Antibody Ratio” or “DAR”) of 2, 3, 4, 5, 6, 7, 8, 9, or 10, respectively. Accordingly, in some embodiments, a vcMMAE-antibody conjugate (e.g., a CEACAM5-ADC) is provided as set forth above, wherein a vcMMAE to antibody or antigen-binding fragment thereof ratio is 1, 2, 3, 4, 5, 6, 7, 8, 9, or 10. In certain exemplary embodiments, a vcMMAE-antibody conjugate (e.g., a CEACAM5-ADC) is provided as set forth above, wherein Ab may include an anti-CEACAM5 antibody (e.g., SG-105.2E4 or SG-105.2E4 EC antibody), and wherein p is 4, representing a vcMMAE to antibody or antigen-binding fragment thereof ratio of 4. Accordingly, in certain exemplary embodiments, a vcMMAE-antibody conjugate (e.g., a CEACAM5-ADC) is provided as set forth above, wherein a vcMMAE to antibody or antigen-binding fragment thereof ratio is 4.

In certain exemplary embodiments, the antibody of the CEACAM5-ADC comprises VH CDR1, VH CDR2, VH CDR3, VL CDR1, VL CDR2, and/or VL CDR3 as set forth in SEQ ID NOs: 8-13, respectively. In certain exemplary embodiments, the antibody of the CEACAM5-ADC comprises CDRs from a VH as set forth in SEQ ID NO: 14 and/or CDRs from a VL as set forth in SEQ ID NO: 15. In certain exemplary embodiments, the antibody of the CEACAM5-ADC comprises a VH as set forth in SEQ ID NO: 14 and/or a VL as set forth in SEQ ID NO: 15. In other embodiments, the antibody of the CEACAM5-ADC comprises a VH/VL pair of SEQ ID NO: 14/SEQ ID NO: 15. In other embodiments, the antibody of the CEACAM5-ADC comprises a VH that has at least about 80% homology or identity (e.g., 80%, 85%, 90%, 91%, 92%, 93%, 94%, 95%, 96%, 97%, 98% or 99%) to SEQ ID NO: 14 and/or comprises a VL that has at least about 80% homology or identity (e.g., 80%, 85%, 90%, 91%, 92%, 93%, 94%, 95%, 96%, 97%, 98% or 99%) to SEQ ID NO: 15. In other embodiments, the antibody of the CEACAM5-ADC comprises a heavy chain that has at least about 80% homology or identity (e.g., 80%, 85%, 90%, 91%, 92%, 93%, 94%, 95%, 96%, 97%, 98% or 99%) to SEQ ID NO: 18 and/or comprises a light chain that has at least about 80% homology or identity (e.g., 80%, 85%, 90%, 91%, 92%, 93%, 94%, 95%, 96%, 97%, 98% or 99%) to SEQ ID NO: 19. In other embodiments, the antibody of the CEACAM5-ADC comprises a heavy chain comprising the amino acid sequence of SEQ ID NO: 18 or 31 and/or comprises a light chain comprising the amino acid sequence of SEQ ID NO: 19. In other embodiments, the antibody of the CEACAM5-ADC comprises a heavy chain that has at least about 80% homology or identity (e.g., 80%, 85%, 90%, 91%, 92%, 93%, 94%, 95%, 96%, 97%, 98% or 99%) to SEQ ID NO: 22 or 33 and/or comprises a light chain that has at least about 80% homology or identity (e.g., 80%, 85%, 90%, 91%, 92%, 93%, 94%, 95%, 96%, 97%, 98% or 99%) to SEQ ID NO: 23. In other embodiments, the antibody of the CEACAM5-ADC comprises a heavy chain comprising the amino acid sequence of SEQ ID NO: 22 or 33 and/or comprises a light chain comprising the amino acid sequence of SEQ ID NO: 23.

Antibodies or ADCs of the ADCs described herein (e.g., anti-CEACAM5 antibodies) can be expressed in a modified form. For instance, a region of additional amino acids, particularly charged amino acids, can be added to the N-terminus of an antibody or antibody of an ADC (e.g., a CEACAM5-ADC) to improve stability and persistence in the host cell, during purification, or during subsequent handling and storage. Also, peptide moieties can be added to an antibody or antibody of an ADC (e.g., a CEACAM5-ADC) of the present invention to facilitate purification. Such regions can be removed prior to final preparation of an antibody molecule or at least one fragment thereof. Such methods are described in many standard laboratory manuals, such as Sambrook, supra; Ausubel, et al., ed., Current Protocols In Molecular Biology, John Wiley & Sons, Inc., NY, N.Y. (1987-2001).

In some embodiments, the CEACAM5-ADC mediates ADCC activity against CEACAM5-expressing cells. In some embodiments, the CEACAM5-ADC thereof does not mediate CDC activity against CEACAM5-expressing cells.

### Therapeutic, Diagnostic, and Other Methods

The antibodies and ADCs of the present invention are useful in various applications including, but are not limited to, therapeutic treatment methods and diagnostic treatment methods.

In one aspect, the invention provides a method for treating cancer. In another aspect, the invention further provides an antibody or pharmaceutical composition as described herein for use in the described method of treating cancer. The invention also provides the use of an antibody as described herein in the manufacture of a medicament for treating cancer.

In some embodiments, the method of treating cancer in a subject comprises administering to the subject in need thereof an effective amount of a pharmaceutical composition comprising any of the CEACAM5 antibodies or ADCs as described herein. In some embodiments, provided is a method of reducing tumor burden or tumor size in a subject, comprising administering to the subject in need thereof an effective amount of a composition comprising an antibody or ADC provided herein. In some embodiments, an antibody or ADC provided herein can be administered to subjects at a level that inhibits cancer cell growth, while at the same time is tolerated by the subject.

In another aspect, provided is a method of one or more of detecting, diagnosing, or monitoring cancer. For example, the anti-CEACAM5 antibodies as described herein can be labeled with a detectable moiety such as an imaging agent and an enzyme-substrate label. The antibodies as described herein can also be used for in vivo diagnostic assays, such as in vivo imaging (e.g., PET or SPECT), or a staining reagent.

With respect to all methods described herein, reference to anti-CEACAM5 antibodies and CEACAM5-ADCs also includes pharmaceutical compositions comprising the anti-CEACAM5 antibodies or CEACAM5-ADCs and one or more additional agents.

The present disclosure also provides methods of treating disorders associated with cells that express CEACAM5, e.g., cancers. In one aspect, the invention provides the use of anti-CEACAM5 antibodies or ADCs thereof (e.g., CEACAM5-ADCs) for the treatment of cancers, such as a solid tumor. In one embodiment, the cancer is pancreatic cancer, lung cancer (e.g., non-small cell lung cancer (NSCLC)), gastric cancer or esophageal cancer. In one aspect, the invention provides the use of anti-CEACAM5 antibodies or conjugates thereof (e.g., CEACAM5-ADCs) for the treatment of solid cancers. In one embodiment, the invention provides the use of anti-CEACAM5 antibodies or conjugates thereof (e.g., CEACAM5-ADCs) for the treatment of pancreatic cancer. In one embodiment, the invention provides the use of anti-CEACAM5 antibodies or conjugates thereof (e.g., CEACAM5-ADCs) for the treatment of lung cancer (e.g., NSCLC). In one embodiment, the invention provides the use of anti-CEACAM5 antibodies or conjugates thereof (e.g., CEACAM5-ADCs) for the treatment of gastric cancer. In one embodiment, the invention provides the use of anti-CEACAM5 antibodies or conjugates thereof (e.g., CEACAM5-ADCs) for the treatment of esophageal cancer.

In some embodiments, provided is a composition comprising an anti-CEACAM5 antibody or ADC thereof (e.g., CEACAM5-ADCs) described herein for use in treatment of a cancer. In some embodiments, provided is a composition comprising a human anti-CEACAM5 antibody or ADC thereof (e.g., CEACAM5-ADCs) described herein in the manufacture of a medicament for treatment of a cancer. In one embodiment, the cancer is a solid tumor. In some embodiments, the cancer is pancreatic cancer, lung cancer (e.g., NSCLC), gastric cancer or esophageal cancer.

In one aspect, the invention provides the use of anti-CEACAM5 antibodies or ADCs thereof (e.g., CEACAM5-ADCs) in combination with an immune checkpoint inhibitor for the treatment of cancers, such as a solid tumor. In one embodiment, the cancer is pancreatic cancer, lung cancer (e.g., NSCLC), gastric cancer or esophageal cancer. In some embodiments, provided is a composition comprising a CEACAM5 antibody or ADC thereof (e.g., CEACAM5-ADC) described herein, and an immune checkpoint inhibitor. In some embodiments, provided is a composition comprising a CEACAM5 antibody or ADC thereof (e.g., CEACAM5-ADC) described herein for use in treatment of a cancer, wherein the CEACAM5 antibody or ADC thereof is for use in combination with an immune checkpoint inhibitor. In some embodiments, provided are uses of a composition comprising a CEACAM5 antibody or ADC thereof (e.g., CEACAM5-ADC) described herein in the manufacture of a medicament for treatment of a cancer, wherein the medicament is for use in combination with an immune checkpoint inhibitor. In some embodiments, provided are uses of a composition comprising a CEACAM5 antibody or ADC thereof (e.g., CEACAM5-ADC) described herein described herein and an immune checkpoint inhibitor in the manufacture of a medicament for treatment of a cancer.

Exemplary immune checkpoint inhibitor is targeted to, without limitation, PD-1, PD-L1, CTLA-4, LAG3, TIM-3, TIGIT, VISTA, TIM1, or BTLA. In some embodiments, the immune checkpoint inhibitor is targeted to one or more of PD-1, PD-L1, CTLA-4, LAG3, TIM-3, TIGIT, VISTA, TIM1, or BTLA. In some embodiments, the immune checkpoint inhibitor is one or more of: an antibody that binds to PD-1, an antibody that binds PD-L1, an antibody that binds CTLA-4, an antibody that binds LAG3, an antibody that binds TIM-3, an antibody that binds TIGIT, an antibody that binds VISTA, an antibody that binds TIM-1, or an antibody that binds BTLA. In some embodiments, the immune checkpoint inhibitor is targeted to one or more of PD-1, PD-L1, CTLA-4, or TIGIT. In some embodiments, the immune checkpoint inhibitor is target to PD-1. In some embodiments, the immune checkpoint inhibitor is one or more of: an antibody that binds to PD-1, an antibody that binds PD-L1, an antibody that binds CTLA-4, or an antibody that binds TIGIT. In some embodiments, the immune checkpoint inhibitor an antibody that binds to PD-1. In some embodiments, the immune checkpoint inhibitor is an anti-PD-1 antibody, such as one or more of: nivolumab, pembrolizumab, cemiplimab, dostarlimab, sasanlimab, and retifanlimab.

In one aspect, the invention provides the use of an anti-CEACAM5 antibody or ADC thereof (e.g., CEACAM5-ADC) described herein in combination with a PD-1 inhibitor (e.g., an anti-PD-1 antibody) for the treatment of cancers, such as a solid tumor. In one embodiment, the cancer is pancreatic cancer, lung cancer (e.g., non-small cell lung cancer (NSCLC)), gastric cancer or esophageal cancer. In one aspect, the invention provides the use of an anti-CEACAM5 antibody or ADC thereof (e.g., CEACAM5-ADC) described herein in combination with a PD-1 inhibitor (e.g., anti-PD-1 antibody) for the treatment of a solid tumor. In one embodiment, the invention provides the use of anti-CEACAM5 antibodies or conjugates thereof (e.g., CEACAM5-ADCs) for the treatment of pancreatic cancer. In one aspect, the invention provides the use of an anti-CEACAM5 antibody or ADC thereof (e.g., CEACAM5-ADC) described herein in combination with a PD-1 inhibitor (e.g., anti-PD-1 antibody) for the treatment of lung cancer (e.g., NSCLC). In one aspect, the invention provides the use of an anti-CEACAM5 antibody or ADC thereof (e.g., CEACAM5-ADC) described herein in combination with a PD-1 inhibitor (e.g., anti-PD-1 antibody) for the treatment of gastric cancer. In one aspect, the invention provides the use of an anti-CEACAM5 antibody or ADC thereof (e.g., CEACAM5-ADC) described herein in combination with a PD-1 inhibitor (e.g., anti-PD-1 antibody) for the treatment of esophageal cancer.

In certain exemplary embodiments, the present invention provides a method for treating cancer in a cell, tissue, organ, animal or patient. In certain exemplary embodiments, the present invention provides a method for treating solid tumors, such as, e.g., pancreatic cancer, lung cancer, gastric cancer and esophageal cancer in a human. In a particular exemplary embodiment, the pancreatic cancer, lung cancer, gastric cancer or esophageal cancer is locally advance or metastatic. In certain exemplary embodiments, the present invention provides a method for treating solid tumors. In certain exemplary embodiments, the present invention provides a method for treating pancreatic cancer. In certain exemplary embodiments, the present invention provides a method for lung cancer (e.g., NSCLC). In certain exemplary embodiments, the present invention provides a method for gastric cancer. In certain exemplary embodiments, the present invention provides a method for esophageal cancer.

In some embodiments, the subject has been previously treated for cancer, such as pancreatic cancer, lung cancer (e.g., NSCLC), gastric cancer or esophageal cancer. In some embodiments, the subject did not respond to the treatment (e.g., the subject experienced disease progression during treatment). In some embodiments, the subject relapsed after the treatment. In some embodiments, the subject experienced disease progression after the treatment. In some embodiments, the treatment previously administered to the subject was not an anti-CEACAM5 antibody or ADC thereof (e.g., CEACAM5-ADC) as described herein.

In some embodiments, the cancer cell expresses CEACAM5. In some embodiments, the cancer cell does not express CEACAM5. In some embodiments, the cancer cell expresses a higher level of CEACAM5 than a non-diseased cell of the same cell type. In some embodiments, the cancer cell expresses a comparable or lower level of CEACAM5 than a non-diseased cell of the same cell type.

In some embodiments, provided herein is an anti-CEACAM5 antibody for use in the treatment of a cancer, wherein the antibody comprises a VH that has at least about 95% (such as 95%, 97%, 98%, 99%, or 100%) homology or identity to SEQ ID NO: 14 and/or comprises an VL that has at least about 95% (such as 95%, 97%, 98%, 99%, or 100%) homology or identity to SEQ ID NO: 15. In some embodiments, provided herein is a CEACAM5-ADC for use in the treatment of a cancer, wherein the anti-CEACAM5 antibody comprises a VH that has at least about 95% (such as 95%, 97%, 98%, 99%, or 100%) homology or identity to SEQ ID NO: 14 and/or comprises a VL that has at least about 95% (such as 95%, 97%, 98%, 99%, or 100%) homology or identity to SEQ ID NO: 15, and wherein the antibody is conjugated to vcMMAE, wherein the vcMMAE has the structure:

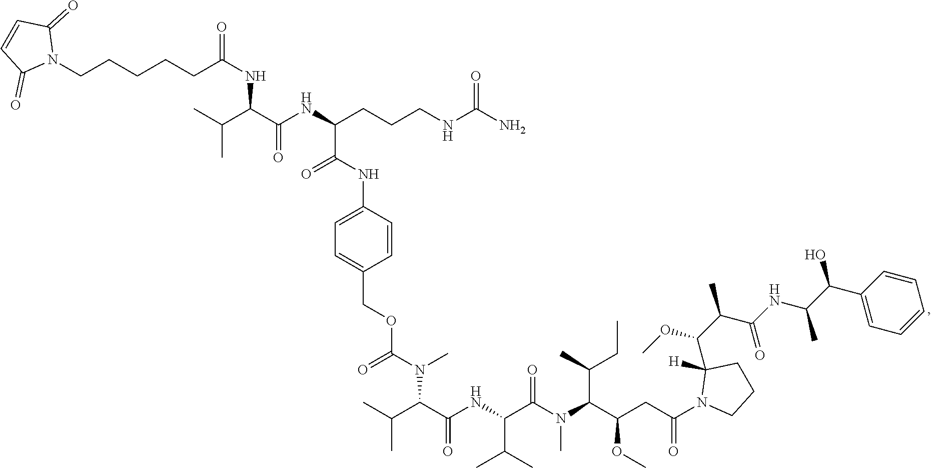 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

In some embodiments, provided is the use of an anti-CEACAM5 antibody or ADC thereof (e.g., a CEACAM5-ADC) described herein for the treatment of a cancer. In some embodiments, provided herein is the use of an anti-CEACAM5 antibody or antibody-drug ADC thereof (e.g., a CEACAM5-ADC) for the treatment of a cancer, wherein the anti-CEACAM5 antibody comprises a VH that has at least about 95% (such as 95%, 97%, 98%, 99%, or 100%) homology or identity to SEQ ID NO: 14 and/or comprises an VL that has at least about 95% (such as 95%, 97%, 98%, 99%, or 100%) homology or identity to SEQ ID NO: 15. In some embodiments, provided is the use of a CEACAM5-ADC for the treatment of a cancer, wherein the anti-CEACAM5 antibody comprises a VH that has at least about 95% (such as 95%, 97%, 98%, 99%, or 100%) homology or identity to SEQ ID NO: 14 and/or comprises a VL that has at least about 95% (such as 95%, 97%, 98%, 99%, or 100%) homology or identity to SEQ ID NO: 15, and wherein the antibody is conjugated to vcMMAE, wherein the vcMMAE has the structure:

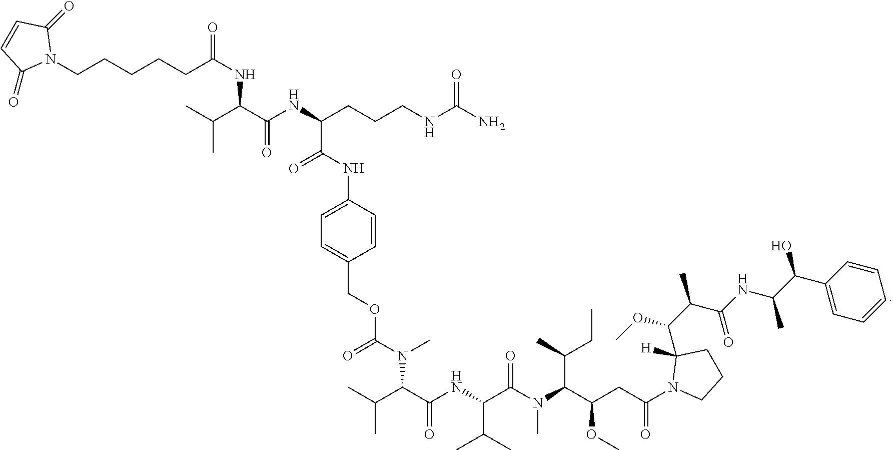 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

In some embodiments, provided is the use of an anti-CEACAM5 antibody or ADC thereof described herein (e.g., a CEACAM5-ADC) in the manufacture of a medicament for the treatment of a cancer. In some embodiments, provided is the use of an anti-CEACAM5 antibody or ADC thereof (e.g., a CEACAM5-ADC) in the manufacture of a medicament for the treatment of a cancer, wherein the anti-CEACAM5 antibody comprises a VH that has at least about 95% (such as 95%, 97%, 98%, 99%, or 100%) homology or identity to SEQ ID NO: 14 and/or comprises a VL that has at least about 95% (such as 95%, 97%, 98%, 99%, or 100%) homology or identity to SEQ ID NO: 15. In some embodiments, provided is the use of a CEACAM5-ADC in the manufacture of a medicament the treatment of a cancer, wherein the anti-CEACAM5 antibody comprises a VH that has at least about 95% (such as 95%, 97%, 98%, 99%, or 100%) homology or identity to SEQ ID NO: 14 and/or comprises a VL that has at least about 95% (such as 95%, 97%, 98%, 99%, or 100%) homology or identity to SEQ ID NO: 15, and wherein the antibody is conjugated to vcMMAE, wherein the vcMMAE has the structure:

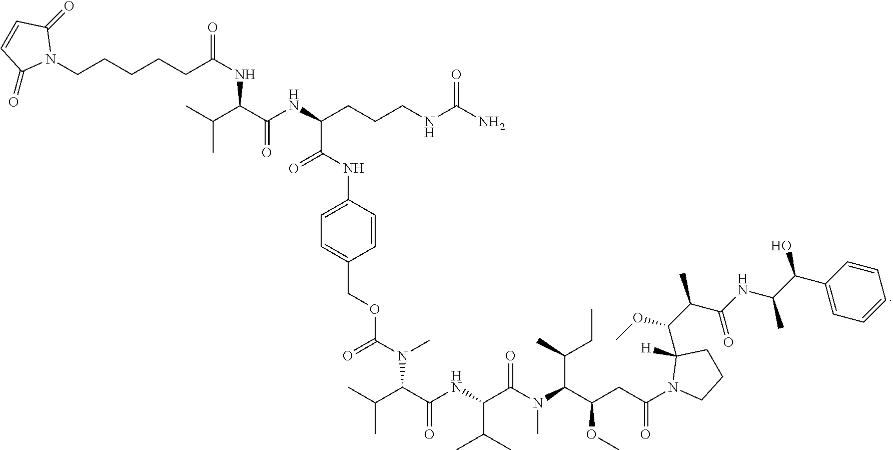 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

In some embodiments, the cancer is a solid tumor. In some embodiments, the cancer is pancreatic cancer, lung cancer (e.g., NSCLC), gastric cancer or esophageal cancer.

In one embodiment of the methods or uses or product for uses provided herein, response to treatment with an antibody or antibody-drug conjugate as described herein, such as e.g., an anti-CEACAM5 antibody or CEACAM5-ADC, is assessed by measuring the size of a tumor derived from the cancer (e.g., pancreatic cancer, lung cancer (e.g., NSCLC), gastric cancer or esophageal cancer). In one embodiment of the methods or uses or product for uses provided herein, response to treatment with a CEACAM5-ADC in combination with a PD-1 inhibitor (e.g., an anti-PD1 antibody) is assessed by measuring the size of a tumor derived from the cancer (e.g., pancreatic cancer, lung cancer, gastric cancer or esophageal cancer).

In one embodiment, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody ADC described herein (e.g., a CEACAM5-ADC). In one embodiment, the size of a tumor derived from the cancer is reduced by at least about 10%-80%. In one embodiment, the size of a tumor derived from the cancer is reduced by at least about 20%-80%. In one embodiment, the size of a tumor derived from the cancer is reduced by at least about 30%-80%. In one embodiment, the size of a tumor derived from the cancer is reduced by at least about 40%-80%. In one embodiment, the size of a tumor derived from the cancer is reduced by at least about 50%-80%. In one embodiment, the size of a tumor derived from the cancer is reduced by at least about 60%-80%. In one embodiment, the size of a tumor derived from the cancer is reduced by at least about 70%-80%. In one embodiment, the size of a tumor derived from the cancer is reduced by at least about 80%. In one embodiment, the size of a tumor derived from the cancer is reduced by at least about 85%. In one embodiment, the size of a tumor derived from the cancer is reduced by at least about 90%. In one embodiment, the size of a tumor derived from the cancer is reduced by at least about 95%. In one embodiment, the size of a tumor derived from the cancer is reduced by at least about 98%. In one embodiment, the size of a tumor derived from the cancer is reduced by at least about 99%. In one embodiment, the size of a tumor derived from the cancer is reduced by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of the antibody or ADC described herein (e.g., a CEACAM5-ADC). In one embodiment, the size of a tumor derived from the cancer is reduced by at least 10%-80%. In one embodiment, the size of a tumor derived from the cancer is reduced by at least 20%-80%. In one embodiment, the size of a tumor derived from the cancer is reduced by at least 30%-80%. In one embodiment, the size of a tumor derived from the cancer is reduced by at least 40%-80%. In one embodiment, the size of a tumor derived from the cancer is reduced by at least 50%-80%. In one embodiment, the size of a tumor derived from the cancer is reduced by at least 60%-80%. In one embodiment, the size of a tumor derived from the cancer is reduced by at least 70%-80%. In one embodiment, the size of a tumor derived from the cancer is reduced by at least 80%. In one embodiment, the size of a tumor derived from the cancer is reduced by at least 85%. In one embodiment, the size of a tumor derived from the cancer is reduced by at least 90%. In one embodiment, the size of a tumor derived from the cancer is reduced by at least 95%. In one embodiment, the size of a tumor derived from the cancer is reduced by at least 98%. In one embodiment, the size of a tumor derived from the cancer is reduced by at least 99%. In one embodiment, the size of a tumor derived from the cancer is reduced by 100%. In one embodiment, the size of a tumor derived from the cancer is measured by magnetic resonance imaging (MRI). In one embodiment, the size of a tumor derived from the cancer is measured by computed tomography (CT). In one embodiment, the size of a tumor derived from the cancer is measured by positron emission tomography (PET). In one embodiment, the size of a tumor derived from the cancer is measured by ultrasound. In some embodiments, the tumor cell expresses CEACAM5. In some embodiments, the tumor cell does not express CEACAM5. In some embodiments, the tumor cell expresses a higher level of CEACAM5 than a non-diseased cell of the same cell type. In some embodiments, the tumor cell expresses a comparable or lower level of CEACAM5 than a non-diseased cell of the same cell type.

In some embodiments, the reduction of tumor size induced by administration of a CEACAM5-ADC comprising an anti-CEACAM5 antibody is at least about any one of: 10%, 20%, 30%, 40%, 50%, 60%, 70%, 80%, 90%, 100%, 2-fold, 3-fold, 5-fold, 10-fold, 15-fold, 20-fold, 30-fold, 50-fold, 100-fold, 200-fold, 500-fold, 1000-fold greater than that induced by the anti-CEACAM5 antibody. In some embodiments, the reduction of tumor size induced by administration of a CEACAM5-ADC comprising an anti-CEACAM5 antibody is at least about 2-fold, 5-fold, 10-fold, or 50-fold greater than that induced by the anti-CEACAM5 antibody.

In some embodiments, a similar reduction of tumor size can be induced by administration of a CEACAM5-ADC comprising an anti-CEACAM5 antibody at a concentration that is at least about any one of: 10%, 20%, 30%, 40%, 50%, 60%, 70%, 80%, 90%, 100%, 2-fold, 3-fold, 5-fold, 10-fold, 15-fold, 20-fold, 30-fold, 100-fold, 200-fold, 500-fold, 1000-fold lower than concentration of administration of the anti-CEACAM5 antibody of antigen-binding fragment thereof. In some embodiments, a similar reduction of tumor size can be induced by administration of a CEACAM5-ADC comprising an anti CEACAM5 antibody of antigen-binding fragment thereof at a concentration that is at least about any one 10-fold lower than the concentration of the anti-CEACAM5 antibody.

In some embodiments, the regression of tumor induced by administration of a CEACAM5-ADC comprising an anti-CEACAM5 antibody is at least about any one of: 10%, 20%, 30%, 40%, 50%, 60%, 70%, 80%, 90%, 100%, 2-fold, 3-fold, 5-fold, 10-fold, 15-fold, 20-fold, 30-fold, 50-fold, 100-fold, 200-fold, 500-fold, 1000-fold greater than that induced by the anti-CEACAM5 antibody. In some embodiments, the regression of tumor induced by administration of a CEACAM5-ADC comprising an anti-CEACAM5 antibody is at least about 50-fold or about 100-fold greater than that induced by the anti-CEACAM5 antibody of antigen-binding fragment thereof.

In some embodiments, a similar regression of tumor can be induced by administration of a CEACAM5-ADC comprising an anti-CEACAM5 antibody at a concentration that is at least about any one of: 10%, 20%, 30%, 40%, 50%, 60%, 70%, 80%, 90%, 100%, 2-fold, 3-fold, 5-fold, 10-fold, 15-fold, 20-fold, 30-fold, 100-fold, 200-fold, 500-fold, 1000-fold lower than the concentration of the anti-CEACAM5 antibody. In some embodiments, a similar regression of tumor can be induced by administration of a CEACAM5-ADC comprising an anti-CEACAM5 antibody at a concentration that is at least about any one 10-fold lower than the concentration of the anti-CEACAM5 antibody.

In one embodiment of the methods or uses or product for uses provided described herein, response to treatment with an antibody or ADC described herein (e.g., a CEACAM5-ADC), promotes regression of a tumor derived from the cancer (e.g., pancreatic cancer, lung cancer (e.g., NSCLC), gastric cancer or esophageal cancer). In one embodiment, a tumor derived from the cancer regresses by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody or ADC described herein (e.g., a CEACAM5-ADC).

In one embodiment, a tumor derived from the cancer regresses by at least about 10% to about 80%. In one embodiment, a tumor derived from the cancer regresses by at least about 20% to about 80%. In one embodiment, a tumor derived from the cancer regresses by at least about 30% to about 80%. In one embodiment, a tumor derived from the cancer regresses by at least about 40% to about 80%. In one embodiment, a tumor derived from the cancer regresses by at least about 50% to about 80%. In one embodiment, a tumor derived from the cancer regresses by at least about 60% to about 80%. In one embodiment, a tumor derived from the cancer regresses by at least about 70% to about 80%. In one embodiment, a tumor derived from the cancer regresses by at least about 80%. In one embodiment, a tumor derived from the cancer regresses by at least about 85%. In one embodiment, a tumor derived from the cancer regresses by at least about 90%. In one embodiment, a tumor derived from the cancer regresses by at least about 95%. In one embodiment, a tumor derived from the cancer regresses by at least about 98%. In one embodiment, a tumor derived from the cancer regresses by at least about 99%. In one embodiment, a tumor derived from the cancer regresses by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of the antibody or ADC described herein (e.g., a CEACAM5-ADC). In one embodiment, regression of a tumor is determined by measuring the size of the tumor by magnetic resonance imaging (MRI). In one embodiment, regression of a tumor is determined by measuring the size of the tumor by computed tomography (CT). In one embodiment, regression of a tumor is determined by measuring the size of the tumor by positron emission tomography (PET). In one embodiment, regression of a tumor is determined by measuring the size of the tumor by ultrasound.

In some embodiments, administration of the CEACAM5-ADC induces an anti-tumor immune response in the subject. In some embodiments, an anti-tumor immune response is determined by a change in a marker of local inflammation at the tumor site. In some embodiments, an anti-tumor response is measured by expression of a chemokine, expression of an interferon, recruitment of a pro-inflammatory immune cell, change in cell cycle marker expression level, or change in transcript level associated with inflammation.

In some embodiments, wherein administration of the CEACAM5-ADC promotes recruitment of innate immune cells and/or adaptive immune cells to a tumor site. In some embodiments, the innate immune cells and/or adaptive immune cells are tumor infiltrating cells. Ins some embodiments, administration of the ADC causes recruitment of dendritic cells to the tumor cite. In some embodiments, administration of the ADC causes recruitment of macrophages to the tumor site. In some embodiments, the presence or absence of cells is determined by immunohistochemistry. In some embodiments, administration of the CEACAM5-ADC promotes recruitment of CD11c+ dendritic cells, F4/80+ macrophages, and/or cells expressing CD86 to a tumor site.

In some embodiments, administration of the ADC causes an increase in gene expression of one or more genes associated with inflammation at the tumor site. In some embodiments, administration of the CEACAM5-ADC causes an increase in expression of a gene associated with responsiveness to PD-1 agents.

In some embodiments, administration of the ADC causes an increase in expression of a dendritic cell and macrophage marker.

In some embodiments, administration of the ADC causes an increase in expression of an MHC class II molecule.

In some embodiments, administration of the ADC causes an increase in expression of a costimulatory molecule. In

In some embodiments, administration of the ADC causes an increase in the presence of inflammatory cells at the tumor site. In some embodiments, the presence of CD3+ cells is increased. In some embodiments, the presence of C4+ cells is increased. In some embodiments, the presence of C8+ cells is increased. In some embodiments, the presence of PD1+ cells is increased. In some embodiments, the presence of inflammatory cells is determined using immunohistochemistry.

In some embodiments, administration of the ADC causes an inflammatory gene expression signature. In some embodiments, administration of the CEACAM5-ADC causes a change in expression of a marker of cell division and/or cell cycle progression.

In some embodiments, a CEACAM5-ADC comprising vcMMAE triggers a more potent response compared to a CEACAM5-ADC with other microtubule inhibitor payload. In some embodiments, the CEACAM5-ADC comprising vcMMAE provided herein triggers a more potent immune response compared to a CEACAM5-ADC comprising a different or same CEACAM5 antibody conjugated to DM1 or DM4. In some embodiments, a lower amount of the CEACAM5-ADC comprising vcMMAE is needed to trigger an immune response compared to a CEACAM5-ADC comprising a different or same CEACAM5 antibody conjugated to DM1 or DM4.

### Pharmaceutical Compositions

In another embodiment, the invention comprises pharmaceutical compositions. A “pharmaceutical composition” refers to a mixture of an antibody or ADC of the invention and one or more excipients.

Pharmaceutical compositions of this invention may be in a variety of forms. These include, for example, liquid, semi-solid and solid dosage forms, such as liquid solutions (e.g., injectable and infusible solutions), dispersions or suspensions, and lyophilized powders. The form depends on the intended mode of administration and therapeutic application.

Other excipients and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pennsylvania, 1975; Liberman et al., Eds., Pharmaceutical Dosage Forms, Marcel Decker, New York, N.Y., 1980; and Kibbe et al., Eds., Handbook of Pharmaceutical Excipients (3rd Ed.), American Pharmaceutical Association, Washington, 1999.

Acceptable excipients are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); or non-ionic surfactants such as TWEEN™, PLURONICS™ or polyethylene glycol (PEG).

### Administration and Dosing

Typically, an antibody or ADC of the invention is administered in an amount effective to treat a condition as described herein. The antibodies or ADCs of the invention can be administered as an antibody or ADC per se, or alternatively, as a pharmaceutical composition containing the antibody or ADC.

The antibodies or ADCs of the invention are administered by any suitable route in the form of a pharmaceutical composition adapted to such a route, and in a dose effective for the treatment intended. In some embodiments, the antibodies or ADCs may be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. In some embodiments, an antibody may be administered subcutaneously. In some embodiments, an antibody or ADC may be administered intravenously. Suitable devices for parenteral administration include needle (including microneedle) injectors (e.g., a “pen”), needle-free injectors, and infusion techniques.

In another embodiment, the antibodies or ADCs of the invention may also be administered topically to the skin or mucosa, that is, dermally or transdermally. In another embodiment, the antibodies or ADCs of the invention can also be administered intranasally or by inhalation. In another embodiment, the antibodies or ADCs of the invention may be administered rectally or vaginally. In another embodiment, the antibodies or ADCs of the invention may also be administered directly to the eye or ear.

The dosage regimen for the antibodies or ADCs of the invention or compositions containing said antibodies is based on a variety of factors, including the type, age, weight, sex and medical condition of the subject; the severity of the condition; the route of administration; and the activity of the particular antibody employed. Thus, the dosage regimen may vary widely.

### Co-Administration

The antibodies or ADCs of the invention can be used alone, or in combination with one or more other therapeutic agents. The invention provides any of the uses, methods or compositions as defined herein wherein an antibody of the invention is used in combination with one or more other therapeutic agent discussed herein.

The administration of two or more agents “in combination” means that all of the agents are administered closely enough in time to affect treatment of the subject. The two or more agents may be administered simultaneously or sequentially. Additionally, simultaneous administration may be carried out by mixing the agents prior to administration or by administering the agents at the same point in time but as separate dosage forms at the same or different site of administration.

### Kits

Another aspect of the invention provides kits comprising the antibody or ADC of the invention or pharmaceutical compositions comprising the antibody or ADC. A kit may include, in addition to the antibody or ADC of the invention or pharmaceutical composition thereof, diagnostic or therapeutic agents. A kit may also include instructions for use in a diagnostic or therapeutic method. In some embodiments, the kit includes the antibody or a pharmaceutical composition thereof and a diagnostic agent. In other embodiments, the kit includes the antibody or ADC or a pharmaceutical composition thereof and one or more therapeutic agents.

In yet another embodiment, the invention comprises kits that are suitable for use in performing the methods of treatment described herein. In one embodiment, the kit contains a first dosage form comprising one or more of the antibodies or ADCs of the invention in quantities sufficient to carry out the methods of the invention. In another embodiment, the kit comprises one or more antibodies or ADCs of the invention in quantities sufficient to carry out the methods of the invention and at least a first container for a first dosage and a second container for a second dosage.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing and method steps.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting. All patents, patent applications and references described herein are incorporated by reference in their entireties for all purposes.

The foregoing description and following Examples detail certain specific embodiments of the disclosure and describes the best mode contemplated by the inventors. It will be appreciated, however, that no matter how detailed the foregoing may appear in text, the disclosure may be practiced in many ways and the disclosure should be construed in accordance with the appended claims and any equivalents thereof.

### SEQUENCE LISTING

TABLE 1
Sequence Listing

| | SEQ | |
|---|---|---|
| | ID | |
| Sequence | NO: | AMINO ACID SEQUENCE |
| Human | 1 | MESPSAPPHRWCIPWQRLLLTASLLTFWNPPTTAKLTIESTPFN |
| CEACAM5 | | VAEGKEVLLLVHNLPQHLFGYSWYKGERVDGNRQIIGYVIGT |
| (hCEACAM | | QQATPGPAYSGREIIYPNASLLIQNIIQNDTGFYTLHVIKSDLVN |
| 5; UniProt | | EEATGQFRVYPELPKPSISSNNSKPVEDKDAVAFTCEPETQDAT |
| Accession | | YLWWVNNQSLPVSPRLQLSNGNRTLTLFNVTRNDTASYKCET |
| #P06731) | | QNPVSARRSDSVILNVLYGPDAPTISPLNTSYRSGENLNLSCHA |
| | | ASNPPAQYSWFVNGTFQQSTQELFIPNITVNNSGSYTCQAHNS |
| | | DTGLNRTTVTTITVYAEPPKPFITSNNSNPVEDEDAVALTCEPEI |
| | | QNTTYLWWVNNQSLPVSPRLQLSNDNRTLTLLSVTRNDVGPY |
| | | ECGIQNELSVDHSDPVILNVLYGPDDPTISPSYTYYRPGVNLSL |
| | | SCHAASNPPAQYSWLIDGNIQQHTQELFISNITEKNSGLYTCQA |
| | | NNSASGHSRTTVKTITVSAELPKPSISSNNSKPVEDKDAVAFTC |
| | | EPEAQNTTYLWWVNGQSLPVSPRLQLSNGNRTLTLFNVTRND |
| | | ARAYVCGIQNSVSANRSDPVTLDVLYGPDTPIISPPDSSYLSGA |
| | | NLNLSCHSASNPSPQYSWRINGIPQQHTQVLFIAKITPNNNGTY |
| | | ACFVSNLATGRNNSIVKSITVSASGTSPGLSAGATVGIMIGVLV |
| | | GVALI |
| Cynomolgus | 2 | MGSPSAPLHRWCIPWQTLLLTASLLTFWNPPTTAQLTIESRPFN |
| monkey | | VAEGKEVLLLAHNVSQNLFGYIWYKGERVDASRRIGSCVIRTQ |
| CEACAM5 | | QITPGPAHSGRETIDFNASLLIHNVTQSDTGSYTIQVIKEDLVNE |
| (cCEACAM | | EATGQFRVYPELPKPYISSNNSNPVEDKDAVALTCEPETQDTT |
| 5; | | YLWWVNNQSLPVSPRLELSSDNRTLTVFNIPRNDTTSYKCETQ |
| XP_0055894 | | NPVSVRRSDPVTLNVLYGPDAPTISPLNTPYRAGENLNLSCHA |
| 91.1)) | | ASNPTAQYFWFVNGTFQQSTQELFIPNITVNNSGSYMCQAHNS |
| | | ATGLNRTTVTAITVYAELPKPYITSNNSNPIEDKDAVTLTCEPE |
| | | TQDTTYLWWVNNQSLSVSSRLELSNDNRTLTVFNIPRNDTTFY |
| | | ECETQNPVSVRRSDPVTLNVLYGPDAPTISPLNTPYRAGENLNL |
| | | SCHAASNPAAQYSWFVNGTFQQSTQELFIPNITVNNSGSYMCQ |
| | | AHNSATGLNRTTVTAITVYVELPKPYISSNNSNPIEDKDAVTLT |
| | | CEPVAENTTYLWWVNNQSLSVSPRLQLSNGNRILTLLSVTRND |
| | | TGPYECGIQNSESAKRSDPVTLNVTYGPDTPIISPPDLSYRSGAN |
| | | LNLSCHSDSNPSPQYSWLINGTLRQHTQVLFISKITSNNNGAYA |
| | | CFVSNLATGRNNSIVKNISVSSGDSAPGSSGLSARATVGIIIGML |
| | | VGVALM |
| hCEACAM | 3 | KLTIESTPFNVAEGKEVLLLVHNLPQHLFGYSWYKGERVDGN |
| 5 | | RQIIGYVIGTQQATPGPAYSGREIIYPNASLLIQNIIQNDTGFYTL |
| Extracellular | | HVIKSDLVNEEATGQFRVYPELPKPSISSNNSKPVEDKDAVAFT |
| Domain | | CEPETQDATYLWWVNNQSLPVSPRLQLSNGNRTLTLFNVTRN |
| (ECD)(35- | | DTASYKCETQNPVSARRSDSVILNVLYGPDAPTISPLNTSYRSG |
| 685) | | ENLNLSCHAASNPPAQYSWFVNGTFQQSTQELFIPNITVNNSGS |
| | | YTCQAHNSDTGLNRTTVTTITVYAEPPKPFITSNNSNPVEDEDA |
| | | VALTCEPEIQNTTYLWWVNNQSLPVSPRLQLSNDNRTLTLLSV |
| | | TRNDVGPYECGIQNELSVDHSDPVILNVLYGPDDPTISPSYTYY |
| | | RPGVNLSLSCHAASNPPAQYSWLIDGNIQQHTQELFISNITEKN |
| | | SGLYTCQANNSASGHSRTTVKTITVSAELPKPSISSNNSKPVED |
| | | KDAVAFTCEPEAQNTTYLWWVNGQSLPVSPRLQLSNGNRTLT |
| | | LFNVTRNDARAYVCGIQNSVSANRSDPVTLDVLYGPDTPIISPP |
| | | DSSYLSGANLNLSCHSASNPSPQYSWRINGIPQQHTQVLFIAKI |
| | | TPNNNGTYACFVSNLATGRNNSIVKSITVSASGTSPGLSA |
| cCEACAM5 | 4 | QLTIESRPFNVAEGKEVLLLAHNVSQNLFGYIWYKGERVDASR |
| ECD(35- | | RIGSCVIRTQQITPGPAHSGRETIDFNASLLIHNVTQSDTGSYTIQ |
| 688) | | VIKEDLVNEEATGQFRVYPELPKPYISSNNSNPVEDKDAVALT |
| | | CEPETQDTTYLWWVNNQSLPVSPRLELSSDNRTLTVFNIPRND |
| | | TTSYKCETQNPVSVRRSDPVTLNVLYGPDAPTISPLNTPYRAGE |
| | | NLNLSCHAASNPTAQYFWFVNGTFQQSTQELFIPNITVNNSGS |
| | | YMCQAHNSATGLNRTTVTAITVYAELPKPYITSNNSNPIEDKD |
| | | AVTLTCEPETQDTTYLWWVNNQSLSVSSRLELSNDNRTLTVF |
| | | NIPRNDTTFYECETQNPVSVRRSDPVTLNVLYGPDAPTISPLNT |
| | | PYRAGENLNLSCHAASNPAAQYSWFVNGTFQQSTQELFIPNIT |
| | | VNNSGSYMCQAHNSATGLNRTTVTAITVYVELPKPYISSNNSN |
| | | PIEDKDAVTLTCEPVAENTTYLWWVNNQSLSVSPRLQLSNGN |
| | | RILTLLSVTRNDTGPYECGIQNSESAKRSDPVTLNVTYGPDTPII |
| | | SPPDLSYRSGANLNLSCHSDSNPSPQYSWLINGTLRQHTQVLFI |
| | | SKITSNNNGAYACFVSNLATGRNNSIVKNISVSSGDSAPGSSGL |
| | | SA |
| hCEACAM | 5 | KLTIESTPFNVAEGKEVLLLVHNLPQHLFGYSWYKGERVDGN |
| 5 N-A1-B1 | | RQIIGYVIGTQQATPGPAYSGREIIYPNASLLIQNIIQNDTGFYTL |
| domains | | HVIKSDLVNEEATGQFRVYPELPKPSISSNNSKPVEDKDAVAFT |
| (NA1B1) | | CEPETQDATYLWWVNNQSLPVSPRLQLSNGNRTLTLFNVTRN |
| (35-320) | | DTASYKCETQNPVSARRSDSVILNVLYGPDAPTISPLNTSYRSG |
| | | ENLNLSCHAASNPPAQYSWFVNGTFQQSTQELFIPNITVNNSGS |
| | | YTCQAHNSDTGLNRTTVTTITVYA |
| hCEACAM | 6 | EPPKPFITSNNSNPVEDEDAVALTCEPEIQNTTYLWWVNNQSL |
| 5 A2-B2 | | PVSPRLQLSNDNRTLTLLSVTRNDVGPYECGIQNELSVDHSDP |
| domains | | VILNVLYGPDDPTISPSYTYYRPGVNLSLSCHAASNPPAQYSWL |
| (A2B2) | | IDGNIQQHTQELFISNITEKNSGLYTCQANNSASGHSRTTVKTIT |
| (321-498) | | VSA |
| hCEACAM | 7 | ELPKPSISSNNSKPVEDKDAVAFTCEPEAQNTTYLWWVNGQSL |
| 5 A3-B3 | | PVSPRLQLSNGNRTLTLFNVTRNDARAYVCGIQNSVSANRSDP |
| domains | | VTLDVLYGPDTPIISPPDSSYLSGANLNLSCHSASNPSPQYSWRI |
| (A3B3) | | NGIPQQHTQVLFIAKITPNNNGTYACFVSNLATGRNNSIVKSIT |
| (499-685) | | VSASGTSPGLSA |
| SG-105.2E4 | 8 | SYAMT |
| VH CDR1 | | |
| SG-105.2E4 | 9 | AISGSVDITYYADSVKG |
| VH CDR2 | | |
| SG-105.2E4 | 10 | DRGNWNYGLFDY |
| VH CDR3 | | |
| SG-105.2E4 | 11 | RASQSISSIYLA |
| VL CDR1 | | |
| SG-105.2E4 | 12 | GASSRAT |
| VL CDR2 | | |
| SG-105.2E4 | 13 | QQFGSSPWT |
| VL CDR3 | | |
| SG-105.2E4 | 14 | EVQLVESGAGLVQPGGSLRLSCAASGFTFSSYAMTWVRQAPG |
| VH | | KGLEWVSAISGSVDITYYADSVKGRFTISRDNSKNTLYLQMNS |
| | | LRAEDTAVYYCAKDRGNWNYGLFDYWGQGTLVTVSS |
| SG-105.2E4 | 15 | EIVLTQSPGTLSLSPGERATLSCRASQSISSIYLAWYQHKPGQAP |
| VL | | MLLIYGASSRATGIPDRFSGSGSGTDFTLTISRLEPEDFAVYYCQ |
| | | QFGSSPWTFGQGTKLEIK |
| SG-105.2E4 | 16 | ASTKGPSVFPLAPSSKSTSGGTAALGCLVKDYFPEPVTVSWNS |
| Heavy chain | | GALTSGVHTFPAVLQSSGLYSLSSVVTVPSSSLGTQTYICNVNH |
| constant | | KPSNTKVDKKVEPKSCDKTHTCPPCPAPELLGGPSVFLFPPKPK |
| region | | DTLMISRTPEVTCVVVDVSHEDPEVKFNWYVDGVEVHNAKT |
| | | KPREEQYNSTYRVVSVLTVLHQDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVMHEALHNHYTQKSLSLSPGK |
| SG-105.2E4 | 17 | RTVAAPSVFIFPPSDEQLKSGTASVVCLLNNFYPREAKVQWKV |
| Light chain | | DNALQSGNSQESVTEQDSKDSTYSLSSTLTLSKADYEKHKVYA |
| constant | | CEVTHQGLSSPVTKSFNRGEC |
| region | | |
| SG-105.2E4 | 18 | EVQLVESGAGLVQPGGSLRLSCAASGFTFSSYAMTWVRQAPG |
| Full-length | | KGLEWVSAISGSVDITYYADSVKGRFTISRDNSKNTLYLQMNS |
| heavy chain | | LRAEDTAVYYCAKDRGNWNYGLFDYWGQGTLVTVSSASTKG |
| (constant | | PSVFPLAPSSKSTSGGTAALGCLVKDYFPEPVTVSWNSGALTSGVH |
| region in | | TFPAVLQSSGLYSLSSVVTVPSSSLGTQTYICNVNHKPSNTKVDKKV |
| italics) | | EPKSCDKTHTCPPCPAPELLGGPSVFLFPPKPKDTLMISRTPEVTC |
| | | VVVDVSHEDPEVKFNWYVDGVEVHNAKTKPREEQYNSTYRVVSVL |
| | | TVLHQDWLNGKEYKCKVSNKALPAPIEKTISKAKGQPREPQVYTL |
| | | PPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLS |
| | | LSPGK |
| SG-105.2E4 | 19 | EIVLTQSPGTLSLSPGERATLSCRASQSISSIYLAWYQHKPGQAP |
| Full-length | | MLLIYGASSRATGIPDRFSGSGSGTDFTLTISRLEPEDFAVYYCQ |
| light chain | | QFGSSPWTFGQGTKLEIKRTVAAPSVFIFPPSDEQLKSGTASVVCL |
| (constant | | LNNFYPREAKVQWKVDNALQSGNSQESVTEQDSKDSTYSLSSTLTL |
| region in | | SKADYEKHKVYACEVTHQGLSSPVTKSFNRGEC |
| italics) | | |
| SG-105.2E4 | 20 | ASTKGPSVFPLAPSSKSTSGGTAALGCLVKDYFPEPVTVSWNS |
| EC Heavy | | GALTSGVHTFPAVLQSSGLYSLSSVVTVPSSSLGTQTYICNVNH |
| chain | | KPSNTKVDKKVEPKSCDKTHTCPPCPAPELLGGPSVFLFPPKPK |
| constant | | DTLMISRTPEVTCVVVDVSHEDPEVKFNWYVDGVEVHNAKT |
| region | | KPREEQYNSTYRVVSVLTVLHQDWLNGKEYKCKVSNKALPA |
| (S400C | | PIEKTISKAKGQPREPQVYTLPPSRDELTKNQVSLTCLVKGFYP |
| bolded and | | SDIAVEWESNGQPENNYKTTPPVLDCDGSFFLYSKLTVDKSR |
| underlined) | | WQQGNVFSCSVMHEALHNHYTQKSLSLSPGK |
| SG-105.2E4 | 21 | RTVAAPCVFIFPPSDEQLKSGTASVVCLLNNFYPREAKVQWKV |
| EC Light | | DNALQSGNSQESVTEQDSKDSTYSLSSTLTLSKADYEKHKVYA |
| chain | | CEVTHQGLSSPVTKSFNRGEC |
| constant | | |
| region | | |
| (S114C | | |
| bolded and | | |
| underlined) | | |
| SG-105.2E4 | 22 | EVQLVESGAGLVQPGGSLRLSCAASGFTFSSYAMTWVRQAPG |
| EC4 Full- | | KGLEWVSAISGSVDITYYADSVKGRFTISRDNSKNTLYLQMNS |
| length heavy | | LRAEDTAVYYCAKDRGNWNYGLFDYWGQGTLVTVSSASTKG |
| chain | | PSVFPLAPSSKSTSGGTAALGCLVKDYFPEPVTVSWNSGALTSGVH |
| (constant | | TFPAVLQSSGLYSLSSVVTVPSSSLGTQTYICNVNHKPSNTKVDKKV |
| region in | | EPKSCDKTHTCPPCPAPELLGGPSVFLFPPKPKDTLMISRTPEVTC |
| italics; | | VVVDVSHEDPEVKFNWYVDGVEVHNAKTKPREEQYNSTYRVVSVL |
| S400C | | TVLHQDWLNGKEYKCKVSNKALPAPIEKTISKAKGQPREPQVYTL |
| bolded and | | PPSRDELTKNQVSLTCLVKGFYPSDIAVEWESNGQPENNYKTTPPV |
| underlined) | | LDCDGSFFLYSKLTVDKSRWQQGNVFSCSVMHEALHNHYTQKSLS |
| | | LSPGK |
| SG-105.2E4 | 23 | EIVLTQSPGTLSLSPGERATLSCRASQSISSIYLAWYQHKPGQAP |
| EC4 Full- | | MLLIYGASSRATGIPDRFSGSGSGTDFTLTISRLEPEDFAVYYCQ |
| length light | | QFGSSPWTFGQGTKLEIKRTVAAPCVFIFPPSDEQLKSGTASVVCL |
| chain | | LNNFYPREAKVQWKVDNALQSGNSQESVTEQDSKDSTYSLSSTLTL |
| (constant | | SKADYEKHKVYACEVTHQGLSSPVTKSFNRGEC |
| region in | | |
| italics; | | |
| S114C | | |
| bolded and | | |
| underlined) | | |
| SG-105.2E4 | 24 | MAWVWTLLFLMAAAQGAQA |
| Heavy | | |
| Chain | | |
| Signal | | |
| Peptide | | |
| SG-105.2E4 | 25 | METDTLLLWVLLLWVPGATG |
| Light Chain | | |
| Signal | | |
| Peptide | | |
| hCEACAM | 26 | MGHLSAPLHR VRVPWQGLLL TASLLTFWNP PTTAQLTTES |
| 1 (GenBank | | MPFNVAEGKE VLLLVHNLPQ QLFGYSWYKG RVDGNRQIV |
| Accession # | | GYAIGTQQAT PGPANSGRET IYPNASLLIQ NVTQNDTGFY |
| NP | | TLQVIKSDLV NEEATGQFHV YPELPKPSIS SNNSNPVEDK |
| 001703.2) | | DAVAFTCEPE TQDTTYLWWI NNQSLPVSPR LQLSNGNRTL |
| | | TLLSVTRNDT GPYECEIQNP VSANRSDPVT LNVTYGPDTP |
| | | TISPSDTYYR PGANLSLSCY AASNPPAQYS WLINGTFQQS |
| | | TQELFIPNIT VNNSGSYTCH ANNSVTGCNR TTVKTIIVTE |
| | | LSPVVAKPQI KASKTTVTGD KDSVNLTCST NDTGISIRWF |
| | | FKNQSLPSSE RMKLSQGNTT LSINPVKRED AGTYWCEVFN |
| | | PISKNQSDPI MLNVNYNALP QENGLSPGAI AGIVIGVVAL |
| | | VALIAVALAC FLHFGKTGRA SDQRDLTEHK PSVSNHTQDH |
| | | SNDPPNKMNE VTYSTLNFEA QQPTQPTSAS PSLTATEIIY |
| | | SEVKKQ |
| hCEACAM | 27 | MGPPSAPPCR LHVPWKEVLL TASLLTFWNP PTTAKLTIES |
| 6 (GenBank | | TPFNVAEGKE VLLLAHNLPQ NRIGYSWYKG ERVDGNSLIV |
| Accession # | | GYVIGTQQAT PGPAYSGRET IYPNASLLIQ NVTQNDTGFY |
| NP | | TLQVIKSDLV NEEATGQFHV YPELPKPSIS SNNSNPVEDK |
| 002474.4) | | DAVAFTCEPE VQNTTYLWWV NGQSLPVSPR LQLSNGNMTL |
| | | TLLSVKRNDA GSYECEIQNP ASANRSDPVT LNVLYGPDVP |
| | | TISPSKANYR PGENLNLSCH AASNPPAQYS WFINGTFQQS |
| | | TQELFIPNIT VNNSGSYMCQ AHNSATGLNR TTVTMITVSG |
| | | SAPVLSAVAT VGITIGVLAR VALI |
| hCEACAM | 28 | MGSPSACPYR VCIPWQGLLL TASLLTFWNL PNSAQTNIDV |
| 7 (GenBank | | VPFNVAEGKE VLLVVHNESQ NLYGYNWYKG ERVHANYRII |
| Accession # | | GYVKNISQEN APGPAHNGRE TIYPNGTLLI QNVTHNDAGI |
| NP | | YTLHVIKENL VNEEVTRQFY VFSEPPKPSI TSNNFNPVEN |
| 008821.2) | | KDIVVLTCQP ETQNTTYLWW VNNQSLLVSP RLLLSTDNRT |
| | | LVLLSATKND IGPYECEIQN PVGASRSDPV TLNVRYESVQ |
| | | ASSPDLSAGT AVSIMIGVLA GMALI |
| hCEACAM | 29 | MGPISAPSCR WRIPWQGLLL TASLFTFWNP PTTAQLTIEA |
| 8 (GenBank | | VPSNAAEGKE VLLLVHNLPQ DPRGYNWYKG ETVDANRRII |
| Accession # | | GYVISNQQIT PGPAYSNRET IYPNASLLMR NVTRNDTGSY |
| NP | | TLQVIKLNLM SEEVTGQFSV HPETPKPSIS SNNSNPVEDK |
| 001807.2) | | DAVAFTCEPE TQNTTYLWWV NGQSLPVSPR LQLSNGNRTL |
| | | TLLSVTRNDV GPYECEIQNP ASANFSDPVT LNVLYGPDAP |
| | | TISPSDTYYH AGVNLNLSCH AASNPPSQYS WSVNGTFQQY |
| | | TQKLFIPNIT TKNSGSYACH TTNSATGRNR TTVRMITVSD |
| | | ALVQGSSPGL SARATVSIMI GVLARVALI |
| SG-105.2E4 | 30 | ASTKGPSVFPLAPSSKSTSGGTAALGCLVKDYFPEPVTVSWNS |
| Heavy chain | | GALTSGVHTFPAVLQSSGLYSLSSVVTVPSSSLGTQTYICNVNH |
| constant | | KPSNTKVDKKVEPKSCDKTHTCPPCPAPELLGGPSVFLFPPKPK |
| region: | | DTLMISRTPEVTCVVVDVSHEDPEVKFNWYVDGVEVHNAKT |
| without C- | | KPREEQYNSTYRVVSVLTVLHQDWLNGKEYKCKVSNKALPA |
| terminal | | PIEKTISKAKGQPREPQVYTLPPSRDELTKNQVSLTCLVKGFYP |
| lysine | | SDIAVEWESNGQPENNYKTTPPVLDSDGSFFLYSKLTVDKSRW |
| | | QQGNVFSCSVMHEALHNHYTQKSLSLSPG |
| SG-105.2E4 | 31 | EVQLVESGAGLVQPGGSLRLSCAASGFTFSSYAMTWVRQAPG |
| Full-length | | KGLEWVSAISGSVDITYYADSVKGRFTISRDNSKNTLYLQMNS |
| heavy chain: | | LRAEDTAVYYCAKDRGNWNYGLFDYWGQGTLVTVSSASTKG |
| without C- | | PSVFPLAPSSKSTSGGTAALGCLVKDYFPEPVTVSWNSGALTSGVH |
| terminal | | TFPAVLQSSGLYSLSSVVTVPSSSLGTQTYICNVNHKPSNTKVDKKV |
| lysine | | EPKSCDKTHTCPPCPAPELLGGPSVFLFPPKPKDTLMISRTPEVTC |
| (constant | | VVVDVSHEDPEVKFNWYVDGVEVHNAKTKPREEQYNSTYRVVSVL |
| region in | | TVLHQDWLNGKEYKCKVSNKALPAPIEKTISKAKGQPREPQVYTL |
| italics) | | PPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLS |
| | | LSPG |
| SG-105.2E4 | 32 | ASTKGPSVFPLAPSSKSTSGGTAALGCLVKDYFPEPVTVSWNS |
| EC Heavy | | GALTSGVHTFPAVLQSSGLYSLSSVVTVPSSSLGTQTYICNVNH |
| chain | | KPSNTKVDKKVEPKSCDKTHTCPPCPAPELLGGPSVFLFPPKPK |
| constant | | DTLMISRTPEVTCVVVDVSHEDPEVKFNWYVDGVEVHNAKT |
| region: | | KPREEQYNSTYRVVSVLTVLHQDWLNGKEYKCKVSNKALPA |
| without C- | | PIEKTISKAKGQPREPQVYTLPPSRDELTKNQVSLTCLVKGFYP |
| terminal | | SDIAVEWESNGQPENNYKTTPPVLDCDGSFFLYSKLTVDKSR |
| lysine | | WQQGNVFSCSVMHEALHNHYTQKSLSLSPG |
| (S400C | | |
| bolded and | | |
| underlined) | | |
| SG-105.2E4 | 33 | EVQLVESGAGLVQPGGSLRLSCAASGFTFSSYAMTWVRQAPG |
| EC4 Full- | | KGLEWVSAISGSVDITYYADSVKGRFTISRDNSKNTLYLQMNS |
| length heavy | | LRAEDTAVYYCAKDRGNWNYGLFDYWGQGTLVTVSSASTKG |
| chain: | | PSVFPLAPSSKSTSGGTAALGCLVKDYFPEPVTVSWNSGALTSGVH |
| without C- | | TFPAVLQSSGLYSLSSVVTVPSSSLGTQTYICNVNHKPSNTKVDKKV |
| terminal | | EPKSCDKTHTCPPCPAPELLGGPSVFLFPPKPKDTLMISRTPEVTC |
| lysine | | VVVDVSHEDPEVKFNWYVDGVEVHNAKTKPREEQYNSTYRVVSVL |
| (constant | | TVLHQDWLNGKEYKCKVSNKALPAPIEKTISKAKGQPREPQVYTL |
| region in | | PPSRDELTKNQVSLTCLVKGFYPSDIAVEWESNGQPENNYKTTPPV |
| italics; | | LDCDGSFFLYSKLTVDKSRWQQGNVFSCSVMHEALHNHYTQKSLS |
| S400C | | LSPG |
| bolded and | | |
| underlined) | | |

### Examples

### Example 1: Anti-CEACAM5 Antibody SG-105.2E4 Generation and Cloning

Human-rodent chimeric anti-CEACAM5 antibodies were derived by repeated immunization of a human immunoglobulin transgenic mouse strain (Alloy Therapeutics) with recombinant protein consisting of the extracellular domain of human CEACAM5 with C-terminal His tag (Acro Biosystems, CE5-5226). B cell responses were confirmed by measuring the serum titer on CEACAM5 expressing cells by flow cytometry.

Splenocytes and lymphocytes from the immunized mice were fused with P3X63.Ag8.653 cells. Following recovery in media, fused cells were plated in semi-solid media in HAT selective media. After culture, IgG secreting hybridoma colonies were identified with a fluorescent secondary antibody and individual colonies were picked into liquid culture. Antibodies secreted from these hybridomas were evaluated for specificity to CEACAM5 and other CEACAM family members. Additionally, hybridoma antibodies were conjugated to a cytotoxic payload to generate ADCs and evaluated for killing cells expressing CEACAM5.

HEK293T cells were stably transfected to express human CEACAM1, cynomolgus CEACAM5, human CEACAM5, human CEACAM6, human CEACAM7, or human CEACAM8. Surface expression was confirmed by positive staining by multiple pan-CEACAM antibody clones by flow cytometry. SG-105.2E4 antibody specificity was evaluated by incubating it with the engineered cell lines, followed by detection with a fluorochrome labelled secondary antibody, and finally running on a flow cytometer. The results are shown as fold signal over background, with background set by a non-binding antibody control in (Table 2). The SG-105.2E4 antibody was found to bind only cells expressing human CEACAM5, but not other CEACAM family members with close sequence homology and not cyno CEACAM5.

TABLE 2
Evaluation of SG-105.2E4 antibody binding to CEACAM family members

| | | | | | | HEK293T |
|---|---|---|---|---|---|---|
| hCEACAM5 | cyCEACAM5 | hCEACAM1 | hCEACAM6 | hCEACAM7 | hCEACAM8 | Parental |
| 18.7 | 1.0 | 0.9 | 0.8 | 1.0 | 0.5 | 1.0 |

The hybridoma, SG-105.2E4 (also referred to as “2E4”), was selected for human CEACAM5 specificity and antibody-drug conjugate (ADC) activity. The variable regions of heavy and light chains were sequenced from the originating hybridoma. These sequences were used as the starting point for synthesizing the fully human antibody. Nucleotide sequences coding for the human SG-105.2E4 heavy chain variable region (SEQ ID NO: 14) and light chain variable region (SEQ ID NO: 15) were joined to the coding regions for human IgG1 containing the S400C mutation and human kappa containing the S114C mutation constant region (SEQ ID NO: 20 and SEQ ID NO: 21, respectively), respectively, to create the fully human antibody (referred herein as “SG-105.2E4 EC4” or “2E4 EC4”), having the amino acid sequence of SEQ ID NO: 22 and SEQ ID NO: 23, for the heavy and light chains, respectively.

### Example 2: Anti-CEACAM5 Antibody SG-105.2E4 and SG-105.2E4 EC4 Binding to CEACAM5

Quantitative binding of the SG-105.2E4 antibody and SG-105.2E4 EC4 antibody to CEACAM5 expressing cells lines, BXPC3, HPAFII, NCIH1395, NCIH573, and NCIH2122 was determined using a huIgG Calibrator kit (Biocytex, cat #CP010) using indirect flow cytometry method. The binding of humanized anti-CEACAM5 antibodies, mAb1 and mAb2, were also tested (the mAb1 EC4 version was tested, in which the constant region of mAb1 was modified to contain the S400C and S114C mutations in its constant regions). Antibodies were used at the saturation concentration of 10 μg/ml. The cell binding, as represented by antibody binding sites/cell, is shown in Table 3 along with RNA levels/expression (last row).

Method: Cells were grown in culture to 80-90% confluency in corresponding medial and lifted from plate using 1× Accutase solution (StemCell Cat #07920) for 10-30 minutes at 37° C. Assay was run in 96 well round bottom plate. 0.2×10⁶ cells were incubated with primary antibodies for 1 hour. Antibody concentration was 10 μg/ml. After incubation and several washes, we added FITC-labelled secondary anti-human antibody from huIgG Calibrator kit at the 1 to 10 dilution and incubated with cells for 15 minutes. At the same time calibration beads were also labelled with same solution of FITC-labelled secondary antibodies for quantitative measurements of antibody binding sites. Following incubation with secondary antibodies, cells and beads were washed several times and fixed with fixative solution provided with huIgG Calibrator kit. Finally, fluorescent signal was measured on the flow cytometer. Linear correlation analysis was done using huIgG calibration kit and number of binding sites/cell were estimated by liner extrapolation using bead calibration curve using Log scale.

TABLE 3
2E4 EC4 Cell Binding (antibody binding sites/cell).

| | BXPC3 | HPAFII | NCIH1395 | NCIH1573 | NCIH2122 |
|---|---|---|---|---|---|
| SG-105.2E4 | 36000 | 328000 | 457000 | 64000 | 52000 |
| SG-105.2E4 | 38000 | 329000 | 470000 | 64000 | 53000 |
| EC4 | | | | | |
| mAb1 EC4 | 35000 | 278000 | 409000 | 74000 | 53000 |
| mAb2 | 25000 | 270000 | 317000 | 47000 | 39000 |
| CEACAM5 | 284 | 652 | 732 | 1285 | 231 |
| RNA (TPM) | | | | | |

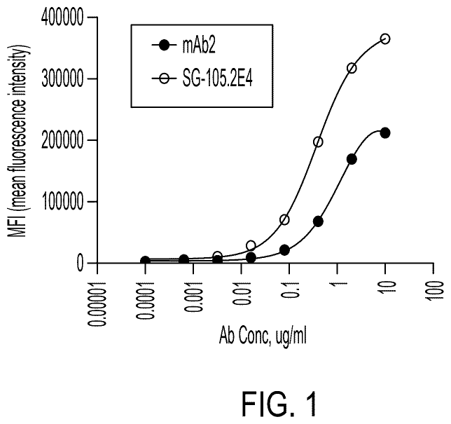

Indirect flow cytometry was also used to determin3 the saturation binding of the mAb2 and SG-105-2E4 antibodies using CEACAM5 positive cells NCIH1395 cells (FIG. 1). Monovalent binding of the SG-105.2E4 antibody and SG-105.2E4 EC antibody to immobilized CEACAM5 recombinant protein was also determined using the Octet HTX system (ForteBio), with the monovalent affinities shown in Table 4.

TABLE 4
Monovalent affinities to human CEACAM5

| CEACAM5 mAb | kD (nM) |
|---|---|
| SG-105.2E4 | 3.0 |
| SG-105.2E4 EC4 | 3.6 |
| mAb1 EC4 | 3.7 |

This example shows the SG-105.2E4 antibody has robust binding to CEACAM5 expressing cells and recombinant protein, as does its counterpart with engineered cysteines, the SG-105.2E4 EC4 antibody.

### Example 3: Anti-CEACAM5 Antibody SG-105.2E4 EC4 Internalization

Incucyte live cell imaging system was used to visualize internalization properties of the SG-105.2E4 EC4 antibody and corresponding controls in a cell-based assay. Intracellular trafficking was performed on cell lines HPAFII and NCIH2122, which express CEACAM5 endogenously. To evaluate internalization, the SG-105.2E4 EC4 and control antibodies were labelled with Incucyte® Human Fabfluor-pH Orange Antibody labeling reagent and then added to live cells to monitor internalization in real time. Fluorescent signal emits upon Fabfluor labeled antibodies reach lysosomes with acidic environment (pH 4.5-5). In the absence of the target or in case non-internalizing surface bound antibodies fluorescent signal remains low. Analysis was performed using Incucyte Adherent Cell-by-Cell module.

Cells were seeded at ˜10000 cells per well in 96-well flat clear bottom black-walled tissue culture-treated microplates (Corning #3603) and left to adhere overnight at 37° C. Test antibodies were incubated with Incucyte® Human Fabfluor-pH Orange Antibody labeling reagent for 30 min at the 1:3 molar ratio and added to cells at the final concentration 0.5 mg/ml.

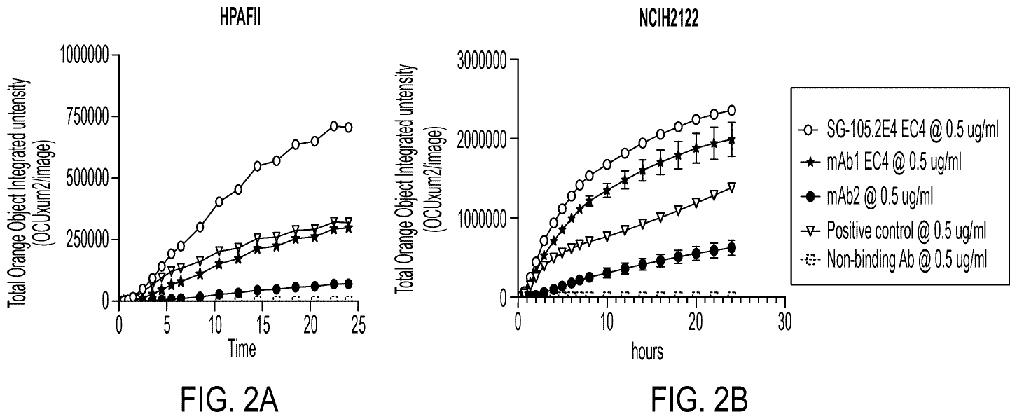

Plates were immediately loaded onto microplate trays in the IncuCyte S5 in a 37° C. incubator. Scans were acquired using the Adherent Cell-by-Cell protocol. Phase data and red channel data (acquisition time set to 400 ms) were collected, with 3 images per well, every 2 to 6 hours for up to 24 hours with the objective set at 10×. Quantification of quenched fluor signal intensity was performed using the IncuCyte software analysis tool. The analysis was refined and tuned per cell line utilizing a label-free cell count and manual image selection for preview and training of the algorithm. Upon completion of analysis, data was calculated using the IncuCyte software with graph metrics set to Total Orange Object Integrated Intensity (ocU mean Object Integrated Intensity) As shown in FIG. 2A and FIG. 2B, the SG-105.2E4 EC4 antibody had good internalization in the HPAFII and NCIH2122 cell lines, with higher rates of internalization than mAb1 EC4 and mAb2. The transferrin receptor antibody was used as a positive control in both cell lines.

### Example 4: Anti-CEACAM5 Antibody SG-105.2E4 Binding to CEACAM5 Regions

The binding of the SG-105.2E4 antibody to the extracellular domain (ECD) of human and cynomolgus (cyno) CEACAM5 (Acro Biosystems, CE5-5226; Sino Biological, 90891-C08H; respectively), and fragments corresponding to the A2 and B2 domains (A2B2) and A3 and B3 domains (A3B3) of human CEACAM5 (see Table 1), was determined by the Octet HTX system (ForteBio), using a bivalent and monovalent method for each. The binding of anti-CEACAM5 antibodies, mAb1 and mAb2, were also tested.

For testing of antibody binding to the ECD of human and cyno CEACAM5 with the monovalent method, the antibodies were diluted in kinetic buffer (0.5% BSA+0.2% Tween20, 1×PBS pH 7.4) and loaded onto AHC (anti-human Fc) biosensors (ForteBio) at 4 μg/ml for 600 seconds. After baselining in kinetic buffer, serial dilutions of recombinant proteins containing a polyhistidine tag (0.5, 1.6, 5.1, 16, 51, 159, and 500 nm for human CEACAM5, and 1.3, 3.4, 9.2, 25, 68, 184, and 500 nm for cyno CEACAM5) in kinetic buffer were allowed to associate with antibody immobilized on biosensors until the top concentration of recombinant protein reached equilibrium with antibody (300 seconds). Then, biosensors were incubated in kinetic buffer to allow for recombinant protein dissociation to occur (1000 seconds). Sensorgrams capturing the association and dissociation of recombinant protein from antibody were generated at 30° C. on an Octet HTX system (Fortebio). Reference biosensors with immobilized antibody were measured in the absence of recombinant protein. Negative control biosensors without immobilized antibody were assessed with recombinant protein present at 500 nm to verify the absence of nonspecific binding of the recombinant proteins to the AHC biosensors themselves. Data for BLI (biolayer interferometry) kinetic experiments were processed and analyzed on Fortebio's Data Analysis HT software. All sensorgrams were processed with a y-axis alignment to an average of the last 5 seconds of the baseline and an inter-step correction aligned to the dissociation step. The affinity constants were calculated by globally fitting the sensorgrams with a 1:1 langmuir adsorption isotherm model (Rmax unlinked) after a reference subtraction of the probe-loaded sensor in absence of analyte.

For testing of antibody binding to the ECD of human and cyno CEACAM5 with the bivalent method, biotinylated recombinant human proteins containing a polyhistidine tag were diluted in kinetic buffer (0.5% BSA+0.2% Tween20, 1×PBS pH 7.4) and loaded onto Ni-NTA (his-chelating) biosensors (Fortebio) at 4 μg/ml for 600 seconds. After baselining in kinetic buffer, serial dilutions of antibody (0.3, 1.1, 3.7, 13, 43, 147, and 500 nm for human CEACAM5 interactions, and 0.7, 2.1, 6.2, 19, 56, 167, and 500 nm for cyno CEACAM5 interactions) in kinetic buffer were allowed to associate with recombinant protein immobilized on biosensors until the top concentration of antibody reached equilibrium with recombinant protein (600 seconds). Then, biosensors were incubated in kinetic buffer to allow for antibody dissociation to occur (1000 seconds). Sensorgrams capturing the association and dissociation of antibody from recombinant protein were generated at 30° C. on an Octet HTX system (Fortebio). Reference biosensors with immobilized recombinant protein were measured in the absence of test article. Negative control biosensors without immobilized recombinant protein were assessed with antibodies present at 500 nm to verify the absence of nonspecific binding of the antibodies to the Ni-NTA biosensors themselves. Data for BLI (biolayer interferometry) kinetic experiments were processed and analyzed on Fortebio's Data Analysis HT software. all sensorgrams were processed with a y-axis alignment to an average of the last 5 seconds of the baseline and an inter-step correction aligned to the dissociation step, the affinity constants were calculated by globally fitting the sensorgrams with a 1:1 langmuir adsorption isotherm model (Rmax unlinked) after a reference subtraction of the probe-loaded sensor in absence of analyte.

For testing of antibody binding to the A2B2 and A3B3 fragments of human CEACAM5 with the monovalent method, the tested antibodies were diluted in immobilization buffer (0.1% BSA+0.02% Tween20, 1×PBS pH 7.4) and loaded onto AHC (anti-human fc) biosensors (Fortebio) at 4 μg/ml for 600 seconds. After baselining in immobilization buffer, then kinetic buffer (0.5% BSA, 0.2% Tween20, 1×PBS pH 7.4), serial dilution of CEACAM5 recombinant proteins containing a c-terminal polyhistidine tag (5000 nm) in kinetic buffer were allowed to associate with antibody immobilized on biosensors until the recombinant protein reached equilibrium with antibody (300 seconds). Then, biosensors were incubated in kinetic buffer to allow for recombinant protein dissociation to occur (1000 seconds). Sensorgrams capturing the association and dissociation of recombinant protein from antibody were generated at 30° C. on an Octet HTX system (Fortebio). Reference biosensors with immobilized recombinant protein were measured in the absence of test article. Data for BLI (biolayer interferometry) kinetic experiments were processed and analyzed on Fortebio's Data Analysis HT software. All sensorgrams were processed with a y-axis alignment to an average of the last 5 seconds of the baseline and an inter-step correction aligned to the dissociation step. The affinity constants were calculated by globally fitting the sensorgrams with a 1:1 langmuir adsorption isotherm model (Rmax unlinked) after a reference subtraction of the probe-loaded sensor in absence of analyte.

For testing of antibody binding to the A2B2 and A3B3 fragments of human CEACAM5 with the bivalent method, CEACAM5 recombinant proteins were diluted in immobilization buffer (0.1% BSA+0.02% Tween20, 1×PBS pH 7.4) and loaded onto Ni-NTA biosensors (Fortebio) at 8 μg/ml for 600 seconds. After baselining in immobilization buffer and then kinetic buffer (0.5% BSA, 0.2% Tween20, 1×PBS pH 7.4), serial dilutions of antibodies (1000 nm or 100 nm) in kinetic buffer were allowed to associate with recombinant protein immobilized on biosensors until the top concentration of antibody reached equilibrium with recombinant protein (600 seconds). Then, biosensors were incubated in kinetic buffer to allow for antibody dissociation to occur (1000 seconds). Sensorgrams capturing the association and dissociation of antibody from recombinant protein were generated at 30° C. on an Octet HTX system (Fortebio). Reference biosensors with immobilized recombinant protein were measured in the absence of test article. Data for BLI (biolayer interferometry) kinetic experiments were processed and analyzed on Fortebio's Data Analysis HT software. All sensorgrams were processed with a y-axis alignment to an average of the last 5 seconds of the baseline and an inter-step correction aligned to the dissociation step. The affinity constants were calculated by globally fitting the sensorgrams with a 1:1 langmuir adsorption isotherm model (Rmax unlinked) after a reference subtraction of the probe-loaded sensor in absence of analyte.

The results showed that the SG-105.2E4 antibody binds to the ECD of human CEACAM5, but not to the ECD of cyno CEACAM5. The results also showed that the SG-105.2E4 antibody does not bind the A2B2 and A2B3 fragments of human CEACAM5. In contrast, mAb1 and mAb2 binds to human and cyno CEACAM5, and mAb1 binds to the A2B2 fragment of human CEACAM5, while mAb2 binds to the A3B3 fragment of human CEACAM5.

### Example 5: Anti-CEACAM5 Antibody SG-105.2E4 Competitive Binding Assay

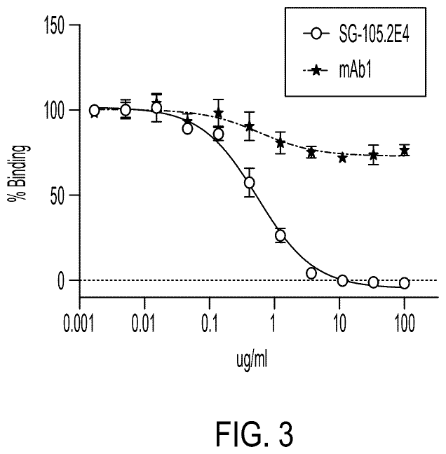

In a competitive binding assay, NCIH2126 cells were preincubated with 11-point dilutions of two different unlabeled CEACAM5 antibodies (SG-105.2E4 and mAb1) and then SG-105.2E4 antibody labeled with Alexa Fluor 488 was added. Signal was measured by flow cytometer and “100%” calculated as only AF488-antibody added, “0” point is calculated when cells were incubated in diluent, without any antibody. As shown in FIG. 3, mAb1 does not compete with SG-105.2E4 antibody.

Method: NCIH2126 CEACAM5 positive cells were lifted from plate with 1×Accutase (StemCell) and 0.2×10⁶ cells were used for each well in 96 well round bottom plate. Cells were pre-incubated with 11 points dilutions of SG-105.2E4 or mAb1 antibodies for 20 min, and then Alexa Fluor 488-labelled SG-105.2E4 antibodies were added at the final concentration 1 μg/ml and incubated for another 60 min. Cells were washed and fixed with 2% paraformaldehyde in PBS. Non-blocked signal was measured on flow cytometry and plotted by GraphPad Prizm.

### Example 6: Anti-CEACAM5 Antibody SG-105.2E4 and Antibody-Drug Conjugate (ADC) in Antibody-Dependent Cellular Cytotoxicity (ADCC) and Antibody-Dependent Cellular Phagocytosis (ADCP) Assays

Human activating FcγRs are divided into three types, FcγRI (CD64), FcγRIIa (CD32a), and FcγRIII (CD16). Upon interaction of the Fc region of the IgG1 antibody backbone with activating FcγRs on innate immune cells, such as monocytes and macrophages, a signaling cascade is triggered to elicit effector functions including ADCC, ADCP, and CDC. NK cells mediate ADCC via FcγRIII, while monocytes/macrophages are thought to mediate ADCP primarily via FcγRI/IIa.

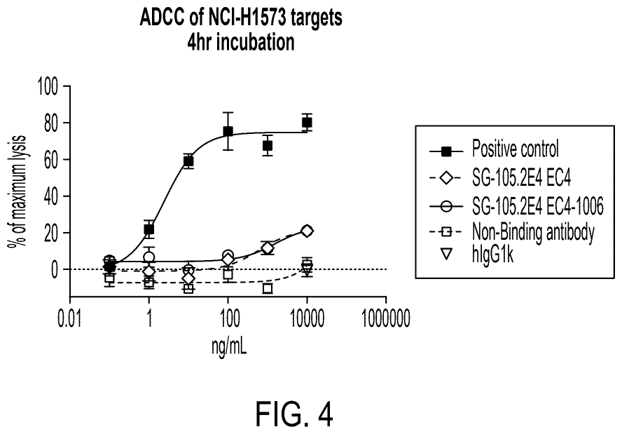

The ability of the SG-105.2E4 EC4 antibody and the SG-105.2E4 EC4 ADC, SG-105.2E4 EC4-1006 (SG-105.2E4 EC4 antibody conjugated to the protease cleavable MMAE/SGD-1006 (vedotin) drug linker) to elicit ADCC were evaluated in primary cell-based assays using DELFIA™ EuTDA Cytotoxicity Detection kit (Revvity, Waltham, Massachusetts). A 4 hr incubation was used in the assay, in which donor BW1282550 NK cells were used as the effector cells, with an effector:target ratio of 10:1. The results are shown in FIG. 4.

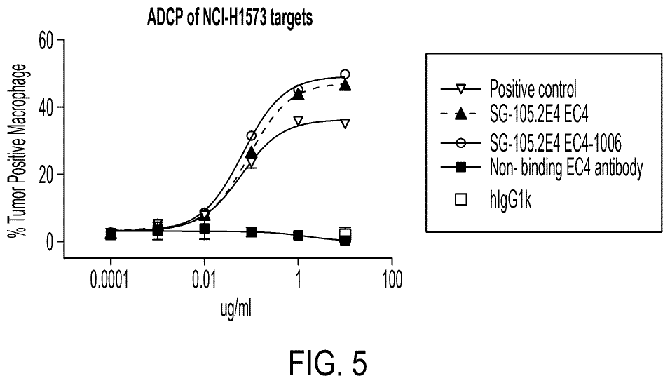

To assess in vitro ADCP, PKH26-labeled target cells were pre-incubated with titrations of ADC or mAb, washed and then monocyte-derived macrophages added to duplicate test wells. Following a 2-hour incubation at 37° C., 5% CO₂, macrophages were labeled with APC-conjugated anti-human CD11c and samples were acquired on a flow cytometer. Phagocytic activity was determined by calculating the percentage of dye-positive macrophages (PKH26+, CD11c+) of total macrophages (CD11c+). Donor 4839BW macrophages were used as the effector cells, at an effector:target ratio of 1:4 effector. The results are shown in FIG. 5.

### Example 7: Anti-CEACAM5 Antibody SG-105.2E4 Specificity for CEACAM5

The SG-105.2E4 antibody was screened for binding against 5861 human plasma membrane proteins and cell surface-tethered secreted proteins and 371 human heterodimers expressed in fixed human HEK293 cells using cell microarray technology (Retrogenix). Appropriate AlexaFluor647 anti-IgG Fc detection antibodies were used to identify gain-of-binding. The SG-105.2E4 antibody was found to have strong intensity interaction with CEACAM5 and no other specific interactions, indicating high specificity for CEACAM5.

### Example 8: Anti-CEACAM5 ADC SG-105.2E4 EC4-1006 in Cytotoxicity Assay

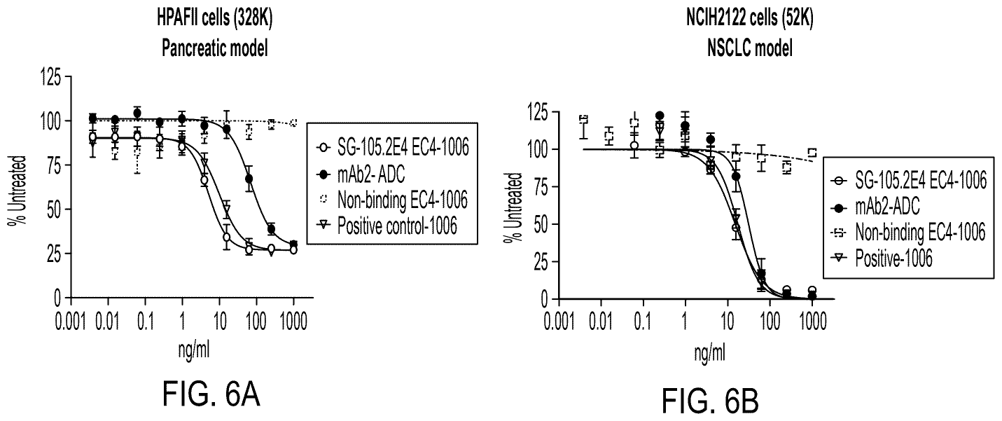

The SG-105.2E4 EC4-1006 ADC was compared to mAb2 ADC (mAb2 conjugated to ravtansine (DM4)) and controls in a cytotoxicity assay. The in vitro CellTiter-Glo® Luminescent Cell Viability Assay was used in determining the cytotoxicity of the SG-105.2E4 EC4-1006 ADC as compared to mAb2 ADC in the CEACAM5-expressing cell lines HPAFII (FIG. 6A) and NCIH2122 (FIG. 6B), and controls, non-binding EC4-1006 (non-binding antibody with constant region modified to contain the S400C and S114C mutations in its constant regions conjugated to vedotin) and positive (control)-1006 (anti-transferrin antibody conjugated to vedotin). The results show the SG-105.2E4 EC4-1006 ADC was similar or better to the positive control and mAb2 ADC.

Method: Cell lines were thawed from cryovials stored at −210° C. into complete growth media and allowed to grow and recover from thaw at 37° C. and 5% CO₂ until cell viabilities as determined by Vi-CELL XR (Beckman Coulter, Indianapolis, IN) were above 90%. Cells were then counted and plated in 40 ul complete growth media in 384-well, white-walled, tissue culture treated plates (Corning). Cell plates were placed at 37° C. and 5% CO₂ overnight to allow cells to equilibrate. ADCs and free drugs were thawed, and 5×10-point serial dilutions were prepared in corresponding cell growth media. Ten microliters of each dilution were then added to each cell plate in quadruplicate. Cells were then left to incubate at 37° C. and 5% CO₂ for 96 hours. Cell plates were then removed from the incubator and allowed to cool to room temperature for 30 minutes. CellTiter-Glo® luminescent assay reagent (Promega Corporation, Madison, WI) was prepared according to Promega's protocol. Ten microliters of CellTiter-Glo® were added to assay plates using a Formulatrix Tempest liquid handler (Formulatrix) and plates were protected from light for 30 minutes at room temperature. The luminescence of each plate was then determined using an En Vision Multimode plate reader (Perkin Elmer, Waltham, MA). Raw data were then analyzed in Graphpad Prism (San Diego, CA) using a nonlinear, 4-parameter curve fit model [Y=Bottom+(Top−Bottom)/(1+10\hat{ }((LogEC50−X)*HillSlope))]. Results are reported as X50 values defined as the concentration of ADC or free drug required to reduce cell viability to 50%.

### Example 9: Anti-CEACAM5 ADC SG-105.2E4 EC4-1006 in Cell Line-Derived Xenograft (CDX) Models

The objective of these studies was to evaluate the anti-tumor activity of the SG-105.2E4 EC4-1006 ADC in vivo in pancreatic (HPAFII) and NSCLC (NCIH2122) cell line-derived xenograft (CDX) models.

Animals were dosed with 3 mg/kg of the SG-105.2E4 EC4-1006 ADC, 3 mg/kg (in the NSCLC model) or 6 mg/kg (in the pancreatic model) mAb2 ADC, and 3 mg/kg of controls (non-binding EC4-1006 or positive control-1006) on Day 0, 7 and 14 (N=5 mice per group) subcutaneously when tumors were 100 mm³. Tumor volume was measured twice weekly until study day 35 or tumor volume reached 1000 mm³.

Female nude mice were implanted with 1×10⁶ HPAFII or NCIH2122 cells in 25% Matrigel HC (Corning #354248) subcutaneously. Once tumor volumes reached 100 mm³, mice were randomized into treatment groups of 5 mice each and dosed every seven days for three total doses (q7dx3). Tumor volumes were measured twice per week, animals were euthanized when tumor volume reached 700-1000 mm³. Stock concentrations of ADC were diluted to desired concentration (with 0.01% Tween20 in PBS) and injected i.p. into each treatment group.

The results are depicted as AUC.3 (Average Slope: area under the curve but above the initial volume (in log-space) minus the area above the curve but below the initial volume (in log-space) divided by the number of days on study squared):

$$AUC \text{.3} - \frac{{AUC}_{log 2} - ( {log}_{2} ( Initial\_Volume ) ∘ Days\_on \_Study )}{Days\_on {\_Study}^{3}} ,$$

adapted from Guo, S., et al., BMC Cancer 19, 718 (2019). The average slope of the growth curve is in log space, with units of log 2 (V)/t. This metric incorporates both the depth and durability of the response, but also accounts for the logarithmic growth of tumors and differences in the initial volume of each tumor. For this metric, negative numbers indicate tumors which, on average, have shrunk from their initial volume.

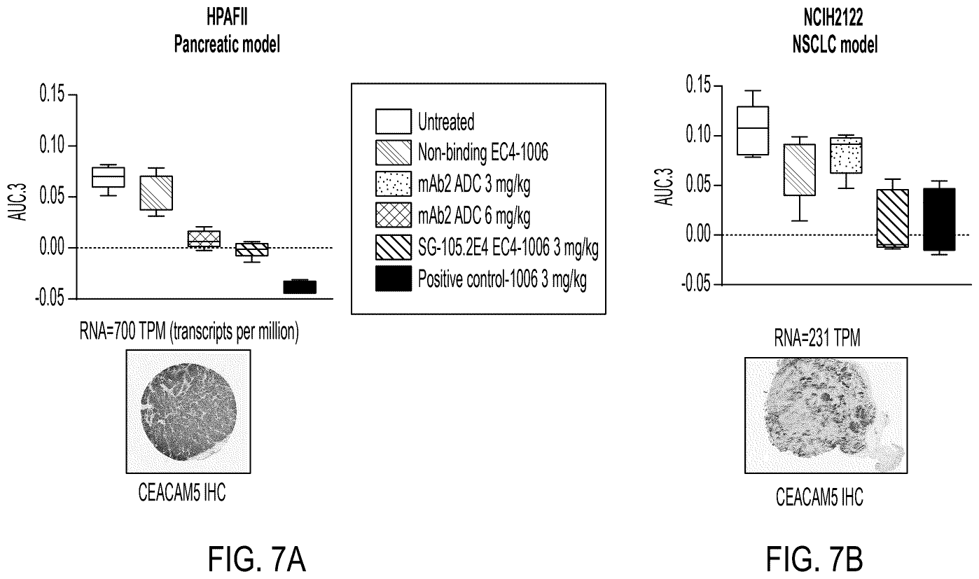

Treatment with 3 mg/kg of the SG-105.2E4 EC4-1006 ADC led to regression of tumor volumes, similar to the mAb2 ADC at 6 mg/kg in the pancreatic model (FIG. 7A), and better than the mAb2 ADC at 3 mg/kg in the NSCLC model (FIG. 7B). CEACAM5 expression on untreated HPAFII and NCIH2122 tumors was evaluated by immunohistochemical staining. As shown in FIG. 7, the tumors exhibited CEACAM5 expression.

### Example 10: Anti-CEACAM5 ADC SG-105.2E4 EC4-1006 in Patient-Derived Xenograft (PDX) Models

The objective of these studies was to evaluate the anti-tumor activity of the SG-105.2E4 EC4-1006 ADC in vivo in a patient-derived xenograft (PDX) model of lung cancer. The SG-105.2E4 EC4-1006 ADC was evaluated at a 3 mg/kg, 3 weekly doses (Day 0, 7, and 14).

Animals were dosed with three weekly doses of 3 mg/kg of the SG-105.2E4 EC4-1006 ADC, 3 mg/kg of non-binding EC4-1006, or 6 mg/kg of mAb2 ADC, intravenously when tumors were 150-300 mm³. Tumor size and body weight were measured twice weekly, and the study was terminated when tumors in the control group reached 1500 mm³ or up to Day 28, whichever occurred first, or maximum up to Day 60.

Specifically, stock mice were bilaterally implanted with fragments from one of the Champions TumorGraft® models representing human lung cancer. After the tumors reached 1000-1500 mm³, they were harvested, and the tumor fragments were implanted subcutaneously (s.c.) in the left flank of the female study mice. Each animal was implanted with a specific passage lot and documented. Tumor growth was monitored twice a week using digital calipers, and the tumor volume (TV) was calculated using the formula (0.52×[length×width²]). When the TV reached approximately 150-300 mm³, animals were matched by tumor size and assigned into control (untreated) or treatment groups (n=5 animals/group). Each animal in the treatment groups was dosed with ADC (3 mg/kg of non-binding EC4-1006, 3 mg/kg of SG-105.2E EC4-1006, or 6 mg/kg of mAb2 ADC) every seven days for three total doses (q7dx3). Tumor size and body weight were measured twice weekly, and the study was terminated when tumors in the control group reached 1500 mm³ or up to Day 28, whichever occurred first, or maximum up to Day 60. Results are depicted as AUC.3, determined as described in Example 9.

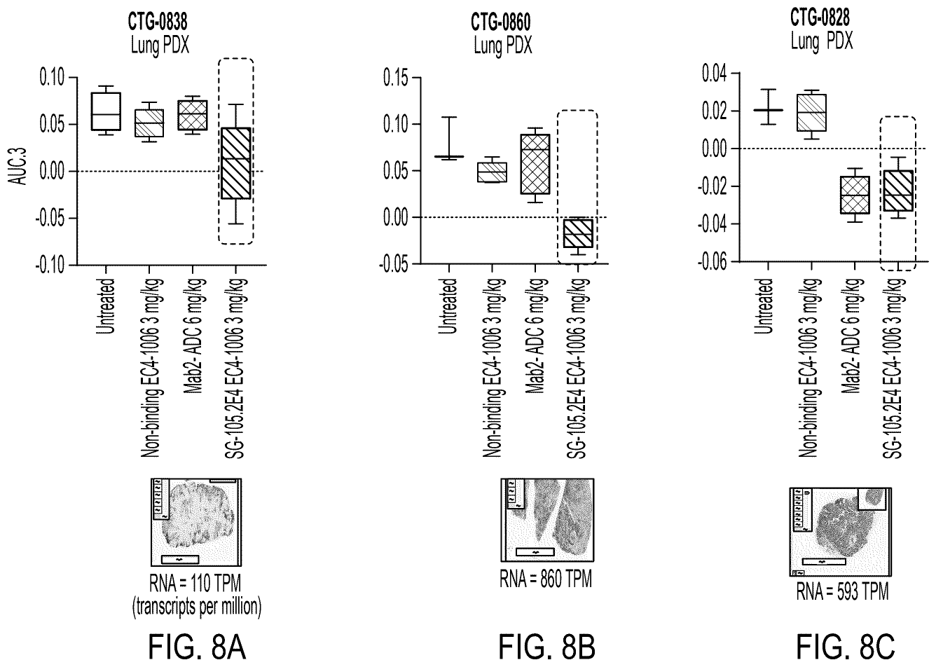

As shown in FIG. 8, treatment with 3 mg/kg of the SG-105.2E4 EC4-1006 ADC led to regression of tumor volumes in all mice, similar to or better than mAb2 ADC at 6 mg/kg (FIG. 8A-8C). CEACAM5 expression on untreated tumors was evaluated by immunohistochemical staining. As shown in FIG. 8, the tumors exhibited a range of CEACAM5 expression levels.

### Example 11: Anti-CEACAM5 ADC SG-105.2E4 EC4-1006 Pharmacokinetics in Nude Mice

The objective of this study was to evaluate pharmacokinetics of SG-105.2E4 EC4-1006 ADC in comparison with mAb1-EC4-1006 ADC (mAb1 EC4 antibody conjugated to the protease cleavable MMAE/SGD-1006 (vedotin) drug linker) following administration of a single intravenous (IV) dose to tumor-bearing (NCIH2122 cell-derived xenograft model) nude female mice. Plasma was collected and analyzed for generic total antibody (gTAb) and for antibody-conjugated drug (acMMAE) by a multiplexed LC-MS/MS assay.

The pharmacokinetic parameters were estimated using a non-compartmental approach in Phoenix WinNonlin 8.2 (Cerata, USA).

TABLE 5
Pharmacokinetic parameters of gTAb in female mouse plasma following
a single IV dose of SG-105.2E4 EC4-1006 ADC or mAb1-EC4-1006 ADC.

| | Dose Level | Cₘₐₓ | AUC_{0-28 d} | t_{1/2} | CL | Vss |
|---|---|---|---|---|---|---|
| Test Article | (mg/kg) | (ng/mL) | (day*ng/mL) | (day) | (mL/day/kg) | (mL/kg) |
| SG-105.2E4 EC4-1006 | 3 | 37100 | 135000 | 3.9 | 21.8 | 128 |
| mAb1-EC4-1006 | 3 | 37500 | 89200 | 3.5 | 33.5 | 155 |

AUC_{0-28 d}: Area under the concentration-time curve from time 0 to 28 days,
Cₘₐₓ: Maximum observed concentration,
T_{1/2}: Terminal elimination half-life,
CL: Clearance,
Vss: Volume of distribution at steady state

TABLE 6
Pharmacokinetic parameters of acMMAE in female mouse plasma following
a single IV dose of SG-105.2E4 EC4-1006 ADC or mAb1-EC4-1006.EC4 ADC

| | ADC Dose Level | Equivalent Dose Level | Cmax | AUC_{0-28 d} | t_{1/2} | CL | Vss |
|---|---|---|---|---|---|---|---|
| Test Article | (mg/kg) | (mg/kg) | (ng/mL) | (day*ng/mL) | (day) | (mL/day/kg) | (mL/kg) |
| SG-105.2E4 EC4-1006 | 3 | 0.0521 | 654 | 1430 | 3.1 | 36.0 | 163 |
| mAb1-EC4-1006 | 3 | 0.0564 | 728 | 1080 | 3.1 | 51.9 | 175 |

AUC_{0-28 d}: Area under the concentration-time curve from time 0 to 28 days,
Cₘₐₓ: Maximum observed concentration,
T_{1/2}: Terminal elimination half-life,
CL: Clearance,
Vss: Volume of distribution at steady state

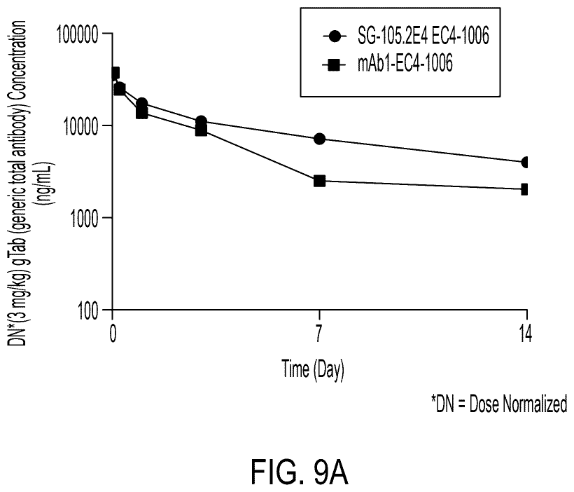

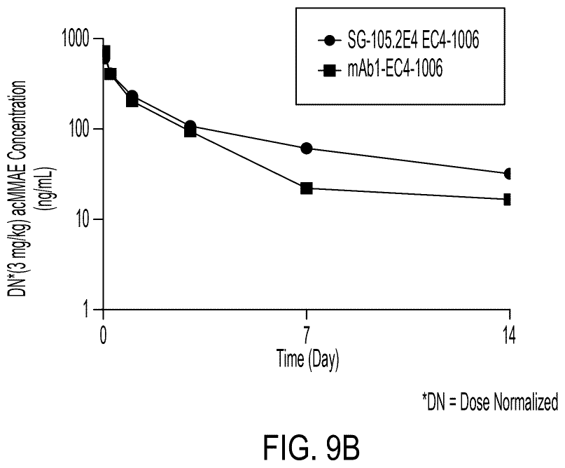

Systemic exposure to gTAb (Table 5, FIG. 9A) and acMMAE (Table 6, FIG. 9B), as assessed by AUC_{0-7d} and AUC_{0-28d} were 1.2 to 1.5-fold higher with the SGD-105.2E4 EC4-1006 ADC compared to mAb1 EC4-1006 ADC at 3 mg/kg.

All references cited herein, including patent applications, patent publications, and scientific literature, are herein incorporated by reference in their entirety, as if each individual reference were specifically and individually indicated to be incorporated by reference.

1. An isolated antibody that binds to human CEACAM5 and comprises a heavy chain variable region (VH) and a light chain variable region (VL), wherein:
g) the VH CDR1 comprises the amino acid sequence of SEQ ID NO: 8,
h) the VH CDR2 comprises the amino acid sequence of SEQ ID NO: 9,
i) the VH CDR3 comprises the amino acid sequence of SEQ ID NO: 10,
j) the VL CDR1 comprises the amino acid sequence of SEQ ID NO: 11,
k) the VL CDR2 comprises the amino acid sequence of SEQ ID NO: 12, and
l) the VL CDR3 comprises the amino acid sequence of SEQ ID NO: 13.

2. The antibody of claim 1, wherein the VH has at least 95% sequence identity to the amino acid sequence of SEQ ID NO: 14, and the VL has at least 95% sequence identity to the amino acid sequence of SEQ ID NO: 15.

3. The antibody of claim 2, wherein the VH comprises the amino acid sequence of SEQ ID NO: 14, and the VL comprises the amino acid sequence of SEQ ID NO: 15.

4. An isolated antibody that binds to human CEACAM5 and comprises a heavy chain variable region (VH) and a light chain variable region (VL), wherein:
c) the VH CDR1, VH CDR2, and VH CDR3 comprises the VH CDR1, VH CDR2, and VH CDR3 amino acid sequences, respectively, of SEQ ID NO: 14; and
d) the VL CDR1, VL CDR2, and VL CDR3 comprises the VL CDR1, VL CDR2, and VL CDR3 amino acid sequences, respectively, of SEQ ID NO: 15,
wherein the CDRs are the Kabat defined CDRs, the Chothia-defined CDRs, or the AbM-defined CDRs.

5. The antibody of claim 1, wherein the antibody further comprises a heavy chain constant region comprising the amino acid sequence of SEQ ID NO: 16, 20, 30 or 32.

6. The antibody of claim 1, wherein the antibody further comprises a light chain constant region comprising the amino acid sequence of SEQ ID NO: 17 or 21.

7. The antibody of claim 1, wherein the antibody comprises a heavy chain comprising the amino acid sequence of SEQ ID NO: 22, and a light chain comprising the amino acid sequence of SEQ ID NO: 23.

8. The antibody of claim 1, wherein the antibody comprises a heavy chain comprising the amino acid sequence of SEQ ID NO: 33, and a light chain comprising the amino acid sequence of SEQ ID NO: 23.

9. The antibody of claim 1, wherein the antibody does not bind to cynomolgus monkey CEACAM5, does not bind the A2-B2 domains of human CEACAM5, does not bind the A3-B3 domains of human CEACAM5, or binds the N, A1, A2 or N-A1-A2 domains of human CEACAM5.

10. An isolated polynucleotide encoding the antibody of claim 1.

11. A pharmaceutical composition comprising a therapeutically effective amount of the antibody of claim 1 and a pharmaceutically acceptable carrier.

12. A method of treating a cancer in a subject, the method comprising administering to the subject in need thereof a therapeutically effective amount of the pharmaceutical composition of claim 11.

13. A CEACAM5 antibody-drug conjugate (CEACAM5-ADC), wherein the CEACAM5-ADC comprises an anti-CEACAM5 antibody conjugated to a vcMMAE (valine-citruline-monomethyl auristatin E), wherein the anti-CEACAM5 antibody comprises a heavy chain variable region (VH) and a light chain variable region (VL), wherein the VH-CDR1, VH-CDR2, VH-CDR3 and VL-CDR1, VL-CDR2, and VL-CDR3 sequences are SEQ ID NOs: 8-13, respectively; wherein the vcMMAE comprises the structure: 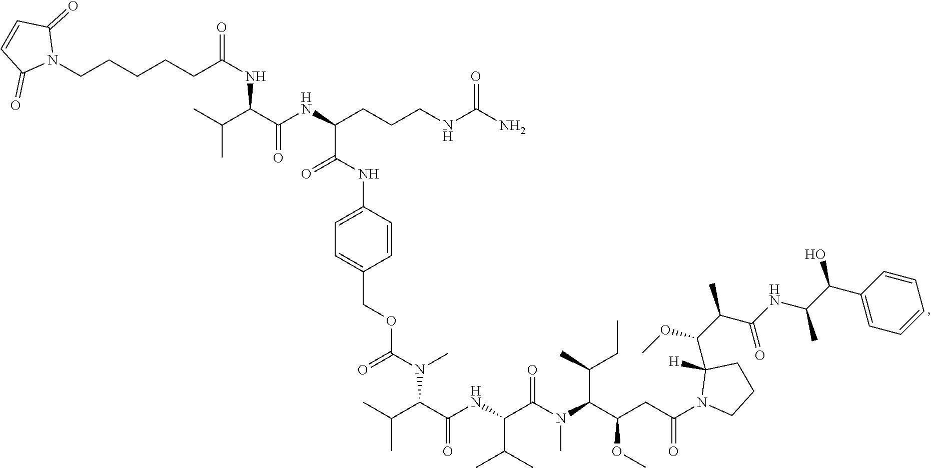 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]
or a pharmaceutically acceptable salt thereof.

14. The CEACAM5-ADC of claim 13, wherein the VH has at least 95% sequence identity to the amino acid sequence of SEQ ID NO: 14, and the VL has at least 95% sequence identity to the amino acid sequence of SEQ ID NO: 15.

15. The CEACAM5-ADC of claim 13, wherein the VH comprises the amino acid sequence of SEQ ID NO: 14, and the VL comprises the amino acid sequence of SEQ ID NO: 15.

16. The CEACAM5-ADC of claim 13, wherein the anti-CEACAM5 antibody comprises a heavy chain comprising the amino acid sequence of SEQ ID NO: 18, 22, 31, or 33, and a light chain comprising the amino acid sequence of SEQ ID NO: 19 or 23.

17. The CEACAM5-ADC of claim 16, wherein the heavy chain comprises the amino acid sequence of SEQ ID NO: 22, and the light chain comprises the amino acid sequence of SEQ ID NO: 23.

18. The CEACAM5-ADC of claim 16, wherein the heavy chain comprises the amino acid sequence of SEQ ID NO: 33, and the light chain comprises the amino acid sequence of SEQ ID NO: 23.

19. The CEACAM5-ADC of claim 15, wherein the CEACAM5-ADC comprises the structure: (a) 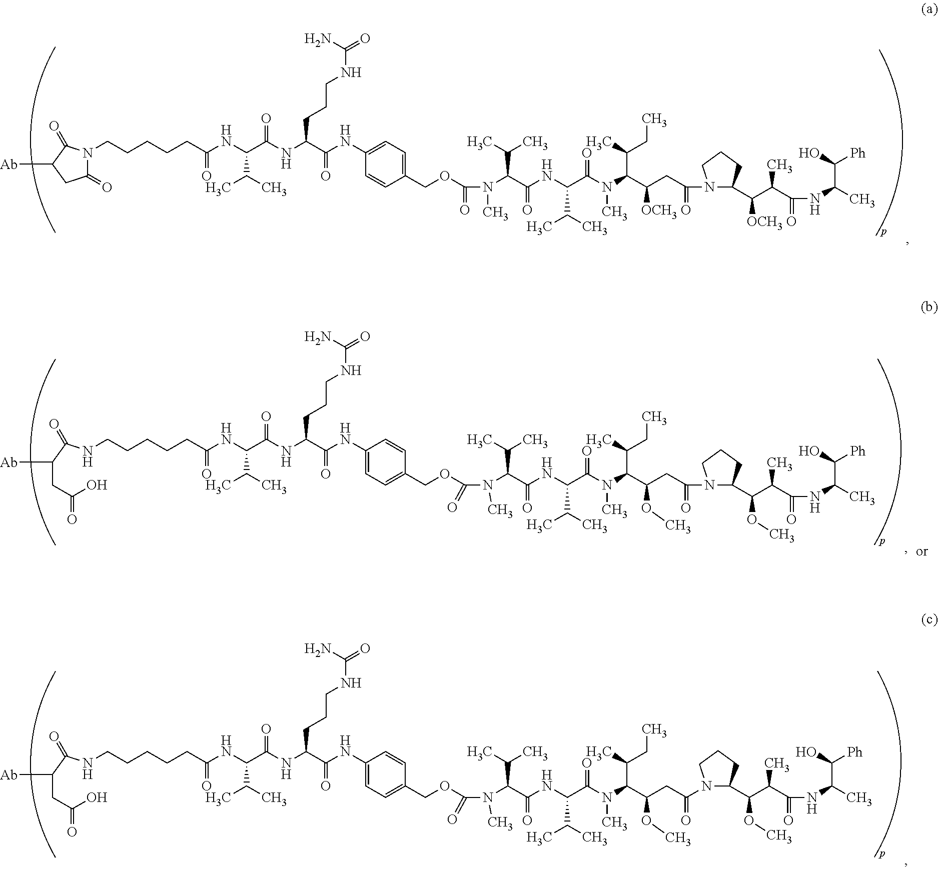
wherein Ab is the anti-CEACAM5 antibody, and p is from about 1 to about 8.

20. The CEACAM5-ADC of claim 19, wherein p is about 4.

21. A pharmaceutical composition comprising a therapeutically effective amount of the CEACAM5-ADC of claim 15, and a pharmaceutically acceptable carrier.

22. A method of treating a cancer in a subject, the method comprising administering to the subject in need thereof a therapeutically effective amount of the pharmaceutical composition of claim 21.


SEQUENCE LISTING (33 sequences)

SEQ ID NO: 1 - protein, 702 aa, synthetic construct
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 2 - protein, 705 aa, synthetic construct
MGSPSAPLHRWCIPWQTLLLTASLLTFWNPPTTAQLTIESRPFNVAEGKEVLLLAHNVSQNLFGYIWYKGERVDASRRIGSCVIRTQQITPGPAHSGRETIDFNASLLIHNVTQSDTGSYTIQVIKEDLVNEEATGQFRVYPELPKPYISSNNSNPVEDKDAVALTCEPETQDTTYLWWVNNQSLPVSPRLELSSDNRTLTVFNIPRNDTTSYKCETQNPVSVRRSDPVTLNVLYGPDAPTISPLNTPYRAGENLNLSCHAASNPTAQYFWFVNGTFQQSTQELFIPNITVNNSGSYMCQAHNSATGLNRTTVTAITVYAELPKPYITSNNSNPIEDKDAVTLTCEPETQDTTYLWWVNNQSLSVSSRLELSNDNRTLTVFNIPRNDTTFYECETQNPVSVRRSDPVTLNVLYGPDAPTISPLNTPYRAGENLNLSCHAASNPAAQYSWFVNGTFQQSTQELFIPNITVNNSGSYMCQAHNSATGLNRTTVTAITVYVELPKPYISSNNSNPIEDKDAVTLTCEPVAENTTYLWWVNNQSLSVSPRLQLSNGNRILTLLSVTRNDTGPYECGIQNSESAKRSDPVTLNVTYGPDTPIISPPDLSYRSGANLNLSCHSDSNPSPQYSWLINGTLRQHTQVLFISKITSNNNGAYACFVSNLATGRNNSIVKNISVSSGDSAPGSSGLSARATVGIIIGMLVGVALM

SEQ ID NO: 3 - protein, 651 aa, synthetic construct
KLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSA

SEQ ID NO: 4 - protein, 654 aa, synthetic construct
QLTIESRPFNVAEGKEVLLLAHNVSQNLFGYIWYKGERVDASRRIGSCVIRTQQITPGPAHSGRETIDFNASLLIHNVTQSDTGSYTIQVIKEDLVNEEATGQFRVYPELPKPYISSNNSNPVEDKDAVALTCEPETQDTTYLWWVNNQSLPVSPRLELSSDNRTLTVFNIPRNDTTSYKCETQNPVSVRRSDPVTLNVLYGPDAPTISPLNTPYRAGENLNLSCHAASNPTAQYFWFVNGTFQQSTQELFIPNITVNNSGSYMCQAHNSATGLNRTTVTAITVYAELPKPYITSNNSNPIEDKDAVTLTCEPETQDTTYLWWVNNQSLSVSSRLELSNDNRTLTVFNIPRNDTTFYECETQNPVSVRRSDPVTLNVLYGPDAPTISPLNTPYRAGENLNLSCHAASNPAAQYSWFVNGTFQQSTQELFIPNITVNNSGSYMCQAHNSATGLNRTTVTAITVYVELPKPYISSNNSNPIEDKDAVTLTCEPVAENTTYLWWVNNQSLSVSPRLQLSNGNRILTLLSVTRNDTGPYECGIQNSESAKRSDPVTLNVTYGPDTPIISPPDLSYRSGANLNLSCHSDSNPSPQYSWLINGTLRQHTQVLFISKITSNNNGAYACFVSNLATGRNNSIVKNISVSSGDSAPGSSGLSA

SEQ ID NO: 5 - protein, 286 aa, synthetic construct
KLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYA

SEQ ID NO: 6 - protein, 178 aa, synthetic construct
EPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSA

SEQ ID NO: 7 - protein, 187 aa, synthetic construct
ELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSA

SEQ ID NO: 8 - protein, 5 aa, synthetic construct
SYAMT

SEQ ID NO: 9 - protein, 17 aa, synthetic construct
AISGSVDITYYADSVKG

SEQ ID NO: 10 - protein, 12 aa, synthetic construct
DRGNWNYGLFDY

SEQ ID NO: 11 - protein, 12 aa, synthetic construct
RASQSISSIYLA

SEQ ID NO: 12 - protein, 7 aa, synthetic construct
GASSRAT

SEQ ID NO: 13 - protein, 9 aa, synthetic construct
QQFGSSPWT

SEQ ID NO: 14 - protein, 121 aa, synthetic construct
EVQLVESGAGLVQPGGSLRLSCAASGFTFSSYAMTWVRQAPGKGLEWVSAISGSVDITYYADSVKGRFTISRDNSKNTLYLQMNSLRAEDTAVYYCAKDRGNWNYGLFDYWGQGTLVTVSS

SEQ ID NO: 15 - protein, 108 aa, synthetic construct
EIVLTQSPGTLSLSPGERATLSCRASQSISSIYLAWYQHKPGQAPMLLIYGASSRATGIPDRFSGSGSGTDFTLTISRLEPEDFAVYYCQQFGSSPWTFGQGTKLEIK

SEQ ID NO: 16 - protein, 330 aa, synthetic construct [sequence omitted]

SEQ ID NO: 17 - protein, 107 aa, synthetic construct [sequence omitted]

SEQ ID NO: 18 - protein, 451 aa, synthetic construct
EVQLVESGAGLVQPGGSLRLSCAASGFTFSSYAMTWVRQAPGKGLEWVSAISGSVDITYYADSVKGRFTISRDNSKNTLYLQMNSLRAEDTAVYYCAKDRGNWNYGLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 215 aa, synthetic construct
EIVLTQSPGTLSLSPGERATLSCRASQSISSIYLAWYQHKPGQAPMLLIYGASSRATGIPDRFSGSGSGTDFTLTISRLEPEDFAVYYCQQFGSSPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDCDGSFFLYSKLTVDKSRWQQGNVFSCSVMHEALHNHYTQKSLSLSPGK

SEQ ID NO: 21 - protein, 107 aa, synthetic construct
RTVAAPCVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 451 aa, synthetic construct
EVQLVESGAGLVQPGGSLRLSCAASGFTFSSYAMTWVRQAPGKGLEWVSAISGSVDITYYADSVKGRFTISRDNSKNTLYLQMNSLRAEDTAVYYCAKDRGNWNYGLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDCDGSFFLYSKLTVDKSRWQQGNVFSCSVMHEALHNHYTQKSLSLSPGK

SEQ ID NO: 23 - protein, 215 aa, synthetic construct
EIVLTQSPGTLSLSPGERATLSCRASQSISSIYLAWYQHKPGQAPMLLIYGASSRATGIPDRFSGSGSGTDFTLTISRLEPEDFAVYYCQQFGSSPWTFGQGTKLEIKRTVAAPCVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 19 aa, synthetic construct
MAWVWTLLFLMAAAQGAQA

SEQ ID NO: 25 - protein, 20 aa, synthetic construct
METDTLLLWVLLLWVPGATG

SEQ ID NO: 26 - protein, 525 aa, synthetic construct
MGHLSAPLHRVRVPWQGLLLTASLLTFWNPPTTAQLTTESMPFNVAEGKEVLLLVHNLPQQLFGYSWYKGRVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

SEQ ID NO: 27 - protein, 344 aa, synthetic construct
MGPPSAPPCRLHVPWKEVLLTASLLTFWNPPTTAKLTIESTPFNVAEGKEVLLLAHNLPQNRIGYSWYKGERVDGNSLIVGYVIGTQQATPGPAYSGRETIYPNASLLIQNVTQNDTGFYTLQVIKSDLVNEEATGQFHVYPELPKPSISSNNSNPVEDKDAVAFTCEPEVQNTTYLWWVNGQSLPVSPRLQLSNGNMTLTLLSVKRNDAGSYECEIQNPASANRSDPVTLNVLYGPDVPTISPSKANYRPGENLNLSCHAASNPPAQYSWFINGTFQQSTQELFIPNITVNNSGSYMCQAHNSATGLNRTTVTMITVSGSAPVLSAVATVGITIGVLARVALI

SEQ ID NO: 28 - protein, 265 aa, synthetic construct
MGSPSACPYRVCIPWQGLLLTASLLTFWNLPNSAQTNIDVVPFNVAEGKEVLLVVHNESQNLYGYNWYKGERVHANYRIIGYVKNISQENAPGPAHNGRETIYPNGTLLIQNVTHNDAGIYTLHVIKENLVNEEVTRQFYVFSEPPKPSITSNNFNPVENKDIVVLTCQPETQNTTYLWWVNNQSLLVSPRLLLSTDNRTLVLLSATKNDIGPYECEIQNPVGASRSDPVTLNVRYESVQASSPDLSAGTAVSIMIGVLAGMALI

SEQ ID NO: 29 - protein, 349 aa, synthetic construct
MGPISAPSCRWRIPWQGLLLTASLFTFWNPPTTAQLTIEAVPSNAAEGKEVLLLVHNLPQDPRGYNWYKGETVDANRRIIGYVISNQQITPGPAYSNRETIYPNASLLMRNVTRNDTGSYTLQVIKLNLMSEEVTGQFSVHPETPKPSISSNNSNPVEDKDAVAFTCEPETQNTTYLWWVNGQSLPVSPRLQLSNGNRTLTLLSVTRNDVGPYECEIQNPASANFSDPVTLNVLYGPDAPTISPSDTYYHAGVNLNLSCHAASNPPSQYSWSVNGTFQQYTQKLFIPNITTKNSGSYACHTTNSATGRNRTTVRMITVSDALVQGSSPGLSARATVSIMIGVLARVALI

SEQ ID NO: 30 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 450 aa, synthetic construct
EVQLVESGAGLVQPGGSLRLSCAASGFTFSSYAMTWVRQAPGKGLEWVSAISGSVDITYYADSVKGRFTISRDNSKNTLYLQMNSLRAEDTAVYYCAKDRGNWNYGLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDCDGSFFLYSKLTVDKSRWQQGNVFSCSVMHEALHNHYTQKSLSLSPG

SEQ ID NO: 33 - protein, 450 aa, synthetic construct
EVQLVESGAGLVQPGGSLRLSCAASGFTFSSYAMTWVRQAPGKGLEWVSAISGSVDITYYADSVKGRFTISRDNSKNTLYLQMNSLRAEDTAVYYCAKDRGNWNYGLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDCDGSFFLYSKLTVDKSRWQQGNVFSCSVMHEALHNHYTQKSLSLSPG